(19) United States
(12) Patent Application Publication
(10) Pub. No.: US 2017/0137895 A1
(43) Pub. Date: May 18, 2017

(54) METHODS OF IDENTIFYING ENZYMES AND MICROORGANISMS

(71) Applicant: ZYMONOSTICS APS, Aarhus C (DK)

(72) Inventors: Magnus Stougaard, Højbjerg (DK); Birgitta Ruth Knudsen, Viby J (DK); Marianne Smedegaard Hede, Aarhus N (DK); Jonas Thomsen, Viby J (DK); Yi-Ping Ho, Aarhus C (DK)

(21) Appl. No.: 15/311,006

(22) PCT Filed: May 15, 2015
(86) PCT No.: PCT/EP2015/060798

§ 371 Date: Nov. 14, 2016

(30) Foreign Application Priority Data
May 16, 2014 (GB) 1408745.6

(51) Int. Cl. C12Q 1/68; C12Q 1/48; C12Q 1/533

(52) U.S. Cl. CPC C12Q 1/6893; C12Q 1/533; C12Q 1/485; C12Q 1/689; G01N 2333/91245

(57) ABSTRACT

The invention provides a method of identifying a microorganism that expresses a nucleic acid-modifying enzyme, in sample, the method comprising: (a) contacting a nucleic acid substrate targeted by the nucleic acid-modifying enzyme with the sample; (b) adding a further nucleic acid molecule to the sample which nucleic acid molecule is ligated to the nucleic acid substrate in the presence of the nucleic acid-modifying enzyme to form a ligation product which comprises a linear single strand of nucleic acid that is capable of being detected; and (c) detecting the presence of the ligation product.

The invention relates to methods of identifying enzymes and microorganisms. Further, the invention relates to methods of diagnosing infectious diseases caused by such microorganisms, and to methods of treatments and compounds for use in the treatments of such infectious disorders.

The listing or discussion of an apparently prior published document in this specification should not necessarily be taken as an acknowledgement that the document is part of the state of the art or is common general knowledge.

Fast and accurate diagnosis is an essential component of malaria control and elimination strategies. The process of diagnosis is initiated by a suspicion of malaria on the basis on clinical criteria and confirmed by a parasitological test: either a blood film for microscopy, a rapid diagnostic test (RDT), polymerase chain reaction (PCR) (World Malaria Report, 2013, ISBN 9789241564694), Quantitative Buffy Coat Assay (QBC) method, or parasite nucleic acids detection. The last three, although technically advanced, are not suitable technologies for quick and inexpensive malaria diagnosis and therefore have not been established on the market for daily diagnosis. Of the commercially available solutions, the leading examples for malaria diagnosis are microscopy and RDT. Accordingly, since 2010, WHO has recommended that suspected cases of malaria should always be confirmed by one of these two methods.

Geographically, and in accordance with the distribution of patients, Africa is the largest and fastest growing market segment for RDTs, followed by South East Asia. The global number of RDTs supplied by manufacturers to the public system increased from 200.000 in 2005 to 108 m in 2012, with most of the RDTs delivered (78%) and used in Africa, followed by the South-East Asia region (16%) and Eastern Mediterranean (3%) [WHO, 2013]. Taking into account that these totals represent public sector distributions and suffer from insufficient reporting (12 out of 44 endemic countries have not reported for 2012) they probably underestimate the real number of RTDs distributed. Accordingly, RTD sales have reached 205 m in 2012. Although impressive, this number far from covers the diagnostic needs on a regional and global level, which WHO conservatively estimates to be over 1 billion.

Due to its high sensitivity level (5-10 parasites/μL) and low test price, microscopic diagnosis is still the ‘golden standard’ for malaria diagnosis. However, its requirement of highly trained technicians and sufficient infrastructure, both rarely available in rural areas, prevents its wide use in resource-limited countries. RDT's introduction to malaria diagnosis is recent and has been aimed to mostly cover areas where microscopic diagnosis is unavailable. Fostered by policy measures, the malaria RDT market has been growing rapidly, and in 2011 well over 155 m RDTs were purchased. RDTs are easy-to-use, do not require any equipment or refrigeration and they are currently commercialized at very low costs (<1 [symbol] ). When performed correctly, good quality RDTs are an excellent tool to diagnose malaria cases at parasitemias over 100 parasites/ul of blood. In the face of economic constraints, RDTs thus represent a valuable tool for disease diagnosis.

In uncomplicated malaria cases, as well as in severe cases, clinical signs and symptoms share commonalities with many other diseases, which results in over-diagnosis and subsequent inappropriate treatment of non-malarial illness with antimalarial drugs. The practice accelerates the evolution of drug resistance and has contributed to the slow progress of malaria control efforts over the past few decades.

Following clinical diagnosis, blood-slide microscopy is the primary diagnostic tool. This method has a high sensitivity detection limit of 5-10 parasites/μL and allows for the specific differentiation of the five malaria parasite species that infect humans. The price of each sample is low (<1 [symbol] ). However, it takes approximately one hour to produce results, and, consistent and dependable microscopy requires specially trained microscopies, electricity and well-maintained and expensive microscopes, which makes it difficult to use in many resource-limited settings.

PCR and nested-PCR are highly sensitive techniques, with very low detection limits of 0.01-1 parasite/μL. This technique is highly specific, being able to differentiate malaria species and identify drug-resistant strains. Both PCR and QBC™ are highly specific for malaria detection. However, due to expensive cost, electric dependence, extensive sample preparation, long readout time, specific training and elaborated laboratory infrastructure requirements, they are unlikely tools for widespread use in malaria endemic areas.

RDTs are immunochromatographic tests that detect parasite antigens in whole blood samples. RDTs can be performed close to the home in settings with no infrastructure or in remote locations. They have 90% sensitivity, specificity for Plasmodium falciparum infection with 200 parasites/μL. RDTs take only 15-20 minutes to provide a result, are simple to perform and can be carried out by non-clinical staff. However, available RDT's still suffer significant drawbacks, namely: variable field performance, degradation at high storage temperatures and no reliability to detect low-density parasitaemia which is useful in assessing the severity of illness and for monitoring a patient's response and avoid overtreatment.

LAMP is a very recent technology for malaria detection developed with the support of FIND (Hopkins et al., JID 2013: 208, 645). The technology is defined by a complex one-step PCR-like method relying on annealing of six primers and subsequent strand displacement amplification (SDA) which is similar to or even more powerful than PCR. Although representing a promising technology, LAMP still holds important drawbacks such as: 1) the DNA target is only present in one, two or a few copies in each pathogen and therefore the initiation of the LAMP reaction can be difficult, 2) it is dependent on electricity and instrumentation for obtaining high incubation temperature and 3) it does not allow internal control(s).

By far the most common diagnostic test used to detect tuberculosis is microscopic examination of stained sputum or other clinical material smeared on a glass slide. When present in sufficiently high concentrations, the bacteria can be identified by a trained technician using this technique, which has changed little since its development over 100 years ago. Such microscopic techniques are associated with numerous disadvantages—they are difficult to perform in the field and require well-trained and motivated technicians but, most-importantly, are insensitive, with only 40-60% test sensitivity under field conditions (WHO report, 2006; Diagnostics for tuberculosis: global demand and market potential; ISBN 9241563303).

Some molecular-based approaches for detecting tuberculosis have been developed, but they are generally expensive and complex, and poorly-suited to performance under field conditions.

For example, several methods have focused on the enzymatic amplification of bacterial DNA or ribosomal RNA (for example, using PCR) and detection of the amplified product by probe-hybridisation. The price and complexity of such approaches has generally limited their use to developed countries, and they display significantly reduced specificity (with a higher proportion of false-positives) when performed under field conditions (WHO report, 2006).

Other molecular-based approaches have focused on estimating cell-mediated immune response of the test individual, by isolating circulating lymphocytes from a blood sample and detecting release of inflammatory cytokines when exposed to mycobacterial antigens. Such assays include Quanti-FERON-TB (by Cellestis Ltd) which was approved by the FDA in 2001, and T-Spot-B (by Oxford Immunotec Limited). However, such approaches are expensive and require the use of fresh blood samples, which can make performance in the field difficult or impossible (WHO report, 2006).

Most of the currently-available HIV tests are based on either detection of HIV-RNA in the individual, or detection of anti-HIV antibodies in the individual.

Tests for detecting HIV-RNA typically involve enzymatic amplification of the target nucleic acid (for example, using Polymerase Chain Reaction-based methods), and detection of the resulting product. Whilst PCR-based methods are generally specific and sensitive, they are also expensive and associated with electric dependence, sample preparation, long readout time, specific training, and laboratory infrastructure requirements, so are inconvenient for use in the field.

All rapid HIV tests currently approved by the FDA are based on the detection of anti-HIV antibodies in the individual. Whilst such approaches can be specific and sensitive, diagnosis is, only possible after a significant immune response has actually been mounted in the individual, and the early stages of HIV infection may therefore be missed.

A disadvantage of such tests is that the inclusion of an enzyme and/or an antibody results in a test kit and test reagents which are poorly stable, particularly in warm climates such as tropical environments.

Group B Streptococcus (GBS), or Streptococcus agalactiae, is a gram-positive bacterium that causes invasive disease primarily in infants, pregnant or postpartum women, and older adults, with the highest incidence among young infants (CDC Morbidity and Mortality Weekly Report, Vol 59, 19 Nov. 2010, No RR-10). The bacterium group B Streptococcus (GBS) emerged as the leading infectious cause of early neonatal morbidity and mortality in the United States. Initial case series reported case-fatality ratios as high as 50%. Maternal colonization with GBS in the genitourinary or gastrointestinal tracts is the primary risk factor for disease. Current detection techniques for Group B Streptococcus are based on antibodies specific for GBS antigens. Again, however, this results in a test kit and test reagents which are poorly stable. Also, more sensitive techniques are required.

The drawbacks of the existing diagnostic methods for malaria, tuberculosis, HIV, and GBS infection outlined above, are not unique to those diseases, but apply equally to the detection methods of other infectious diseases. Hence, there is a need for new detection strategies for malaria, tuberculosis, HIV, GBS infection and other diseases.

The inventors have now found that detecting enzyme activities, and particularly the activities of nucleic acid-modifying enzymes, provides a sensitive detection technique for malaria and other diseases. By definition, enzymes convert substrate molecules to products with changed chemical or physical characteristics without the enzymes themselves being affected by the process. It follows that one enzyme can, in general, create indefinite amounts of product provided that it is fuelled with sufficient substrate. According to the methods of the present invention, therefore, specific enzymes and/or enzymatic activities are detected on the basis of a detection of a nucleic acid substrate, which is specifically targeted and processed by that specific enzymes and/or enzymatic activities. Furthermore, according to the methods of the present invention, a microorganism, cell or cell type is identified in a sample by detecting a nucleic acid substrate which is targeted by a nucleic acid-modifying enzyme system specific for said microorganism, cell or cell type. The detection method also forms the basis of identification and diagnostic methods, compositions and uses of the present invention.

Compared to conventional methods for detecting diseases such as malaria, the present technology has several advantages. For example, the methods do not require any instrumentation and can readily be performed in the context of a strip. The methods are isothermal, work on non-invasive samples such as saliva, and show high sensitivity. Also, the number of molecules of nucleic acid-modifying enzymes present in cells is high (e.g. around 10⁷ molecules of type I topoisomerase are estimated in eukaryotic cells), and so the methods are believed to be less prone to false negatives. Other benefits include equal specificity to PCR, and a simple and practical usability.

Accordingly, a first aspect of the invention provides a method of identifying a microorganism that expresses a nucleic acid-modifying enzyme, in a sample, the method comprising:

(a) contacting a nucleic acid substrate'targeted by the nucleic acid-modifying enzyme with the sample;

(b) adding, a further nucleic acid molecule to the sample which nucleic acid molecule is ligated to the nucleic acid substrate in the presence of the nucleic acid-modifying enzyme to form a ligation product which comprises a linear single strand of nucleic acid that is capable of being detected; and

(c) detecting the presence of the ligation product.

By identifying a microorganism in a sample, we include the meaning of detecting the presence of that microorganism in the sample. The identification may be qualitative whereby the readout is simply whether or not the sample contains that microorganism, or, depending upon how the ligation product is detected in step (d), the identification may be quantitative, as is described further below.

By microorganism that expresses a nucleic acid-modifying enzyme, we include the meaning of any microorganism that naturally, or otherwise, expresses the nucleic acid-modifying enzyme defined herein. Preferably, the microorganism is one that is characterised by the expression of the nucleic-acid-modifying enzyme in question. For example, the microorganism may be the only, microorganism, or one of only a few microorganisms, to express that nucleic acid-modifying enzyme. In order words, the expression of the nucleic acid-modifying enzyme is specific for that microorganism. In this way, detection of the activity of the particular nucleic acid-modifying enzyme can be used as a direct readout of the presence of the microorganism that expresses it.

The microorganism can be any pathogenic microorganism or parasitic microorganism. Examples include a virus, a bacterium, a protozoa, a fungus, a mould, an amoeba or a parasitic worm. Typically, the microorganism is a bacterium or virus or protozoan.

The microorganism may be selected from the group consisting of a Eukaryote, an Alveolate, an Apicomplexan/sporozoan, a Haematozoan, a Haemosporidia, and a Plasmodiidae.

The microorganism can be a bacterium, in which case it may be selected from the group consisting of Eubacteria, Actinobacteria, Actinomycetes, Corynebacterinease, and Mycobacteriaceae.

The microorganism identified by the method of the invention is typically involved in and/or is the causative agent in one or more infectious disorders. For example, the microorganism may be involved in tuberculosis, malaria, HIV infection (e.g. AIDs), SIV infection, MLV infection, FIV infection, Group B Streptococcal disease (e.g. perinatal GBS disease), toxoplasmosis, or Lyme disease/borreliosis (Borrelia). Viruses, such as retroviruses, have been linked to a wide range of cancers, and so the when the microorganism is a virus (e.g. a retrovirus), the disorder may be a cancer. For example leukaemia (e.g. chronic and acute) is known to be associated with retroviral infection.

In a preferred embodiment, the microorganism belongs to the Mycobacteriaceae family, such as one selected from the Mycobacterium genus. The microorganism may be selected from the Mycobacterium tuberculosis complex (MTBC), the members of which are causative agents of human and animal tuberculosis. Species in this complex include Mycobacterium tuberculosis, Mycobacterium bovis, Mycobacterium bovis BCG, Mycobacterium africanum, Mycobacterium canetti, Mycobacterium caprae, Mycobacterium micron and Mycobacterium pinnipedii. Other species include Mycobacterium avium and Mycobacterium smegmatis. Preferably, the microorganism is Mycobacterium tuberculosis.

In another preferred embodiment, the microorganism belongs to the Plasmodiidae family, such as one selected from the Plasmodium genus. Preferably, the microorganism is Plasmodium falciparum, which is a causative agent of human malaria. Other possible species, however, include Plasmodium clelandi, Plasmodium draconis, Plasmodium lionatum, Plasmodium saurocordatum, Plasmodium vastator, Plasmodium juxtanucleare, Plasmodium basillisci, Plasmodium lygosomae, Plasmodium mabuiae, Plasmodium minasense, Plasmodium rhadinurum, Plasmodium volans, Plasmodium anasum, Plasmodium circumflexum, Plasmodium dissanaikei, Plasmodium durae, Plasmodium fallax, Plasmodium, formosanum, Plasmodium gabaldoni, Plasmodium garnhami, Plasmodium gundersi, Plasmodium hegneri, Plasmodium lophurae, Plasmodium pedioecetii, Plasmodium pinnotti, Plasmodium polare, Plasmodium cathemerium, Plasmodium coggeshalli, Plasmodium cotumixi, Plasmodium elongatum, Plasmodium gallinaceum, Plasmodium giovannolai, Plasmodium lutzi, Plasmodium matutinum, Plasmodium paddae, Plasmodium parvalum, Plasmodium relictum, Plasmodium tejera, Plasmodium hermani, Plasmodium floridense, Plasmodium tropiduri, Plasmodium billbrayi, Plasmodium billcollinsi, Plasmodium gaboni, Plasmodium reichenowi, Plasmodium pessoai, Plasmodium tomodoni, Plasmodium wenyoni, Plasmodium ashfordi, Plasmodium bertii, Plasmodium bambusicolai, Plasmodium columbae, Plasmodium corradetti, Plasmodium dissanaikei, Plasmodium globularis, Plasmodium hexamerium, Plasmodium jiangi, Plasmodium kemp, Plasmodium lucens, Plasmodium megaglobularis, Plasmodium multivacuolaris, Plasmodium nucelophilum, Plasmodium papemai, Plasmodium parahexamerium, Plasmodium paranucelophilum, Plasmodium rouxi, Plasmodium vaughani, Plasmodium dominicum, Plasmodium chirichuae, Plasmodium mexicanum, Plasmodium pifanoi, Plasmodium bouillize, Plasmodium brasilianum, Plasmodium ceropitheci, Plasmodium coatneyi, Plasmodium cynomolgi, Plasmodium eylesi, Plasmodium fieldi, Plasmodium fragile, Plasmodium georgesi, Plasmodium girardi, Plasmodium gonderi, Plasmodium gora, Plasmodium gorb, Plasmodium inui, Plasmodium jefferyi, Plasmodium joyeuxi, Plasmodium knowlei, Plasmodium hyobati, Plasmodium malariae, Plasmodium ovale, Plasmodium petersi, Plasmodium pitheci, Plasmodium rhodiani, Plasmodium schweitzi, Plasmodium semiovale, Plasmodium semnopitheci, Plasmodium silvaticum, Plasmodium simium, Plasmodium vivax, Plasmodium youngi, Plasmodium achiotense, Plasmodium adunyinkai, Plasmodium aeuminatum, Plasmodium agamae, Plasmodium balli, Plasmodium beltrani, Plasmodium brumpti, Plasmodium cnemidophoria, Plasmodium diploglossi, Plasmodium giganteum, Plasmodium heischi, Plasmodium josephinae, Plasmodium pelaezi, Plasmodium zonuriae, Plasmodium achromaticum, Plasmodium aegyptensis, Plasmodium anomaluri, Plasmodium atheruri, Plasmodium berghei, Plasmodium booliati, Plasmodium brodeni, Plasmodium bubalis, Plasmodium bucki, Plasmodium caprae, Plasmodium cephalophi, Plasmodium chabaudi, Plasmodium coulangesi, Plasmodium cyclopsi, Plasmodium foleyi, Plasmodium girardi, Plasmodium incertae, Plasmodium inopinatum, Plasmodium landauae, Plasmodium lemuris, Plasmodium melanipherum, Plasmodium narayani, Plasmodium odocoilei, Plasmodium percygarnhami, Plasmodium pulmophilium, Plasmodium sandoshami, Plasmodium traguli, Plasmodium tyrio, Plasmodium uilenbergi, Plasmodium vinckeic, Plasmodium watteni and Plasmodium yoelli.

In yet another preferred embodiment, the microorganism is a retrovirus such as Human Immunodeficiency Virus (HIV), murine leukaemia virus (MLV), simian immunodeficiency virus (SIV) and feline immunodeficiency virus (FIV).

In still yet another preferred embodiment, the microorganism is a Streptococcus such as Group B Streptococcus (GBS), for example Streptococcus agalactiae.

Other possible microorganisms include enterobacteria, enterococci, corynebacteria, Salmonella spp, Paratuberculosis, Brachyspira spp (e.g. Brachyspira hyodysenteriea), Lawsonia intracellularis, Campylobacter spp, Clostridium spp, coronavirus, rotavirus, torovirus, calcivirus, astrovirus, canine parvovirus, coccidian, cryptosporidia, Escherichi coli (e.g. enteropathogenic E coli), Yersinia spp (e.g. Yersinia enterocolitica), Coxiella burnetti, bovine virus, diarrhoea virus, bovine herpes virus, rinderpest virus, sappovirus, norovirus, Vibrio spp (e.g. Vibrio cholera), Shigella spp, and Helicobacter spp.

By a nucleic acid-modifying enzyme, we include the meaning of any enzyme that has nucleic acid as its substrate, and which acts to change the chemical or physical characteristics of the nucleic acid. For example, the enzyme may alter the covalent structure of the nucleic acid.

By ‘nucleic acid’, ‘oligonucleotide’ or ‘polynucleotide’ as used herein, we include the meaning of polynucleotides, such as deoxyribonucleic acid (DNA) or ribonucleic acid (RNA), oligonucleotides, fragments generated by the polymerase chain reaction (PCR), and fragments generated by any of ligation, scission, endonuclease action, and exonuclease action. Polynucleotides can be composed of monomers that are naturally-occurring nucleotides (such as DNA and RNA), or analogs of naturally-occurring nucleotides (e.g. alpha-enantiomeric forms of naturally-occurring nucleotides) or a combination of both. Modified nucleotides can have alterations in sugar moieties and/or in pyrimidine or purine base moieties. Sugar modifications include, for example, replacement of one or more hydroxyl groups with halogens, alkyl groups, amines, and azido groups, or sugars can be functionalized as ethers or esters. Moreover, the entire sugar moiety can be replaced with sterically and electronically similar structures, such as aza-sugars and carbocyclic sugar analogs. Examples of modifications in a base moiety include alkylated purines and pyrimidines, acylated purines or pyrimidines, or other well-known heterocyclic substitutes. Nucleic acid monomers can be linked by phosphodiester bonds or analogs of such linkages. Analogs of phosphodiester linkages include phosphorothioate, phosphorodithioate, phosphoroselenoate, phosphorodiselenoate, phosphoroanilothioate, phosphoranilidate, phosphoramidate, and the like. The term “polynucleotide” also includes so-called “peptide nucleic acids,” which comprise naturally-occurring or modified nucleic acid bases attached to a polyamide backbone. Nucleic acids can be either single stranded or double stranded.

The nucleic acid-modifying enzyme may be a DNA- or RNA-modifying enzyme. Preferably, the nucleic acid-modifying enzyme is a DNA-modifying enzyme.

Examples of nucleic acid-modifying enzymes include nucleases, ligases, recombinases, phosphatases, phosphorylases, integrases, transposases, and topoisomerases (e.g. type I and type II topoisomerases).

It will be appreciated that some enzymes have more than one effect on a nucleic acid, such as both cleavage and ligase activities. It is preferred that the nucleic acid-modifying enzyme has at least ligase activity. Hence in one embodiment, the nucleic acid-modifying enzyme is one that has ligase activity such as a topoisomerase (e.g. a type I topoisomerase), an integrase or a recombinase.

In a preferred embodiment, the nucleic acid-modifying enzyme is a type I topoisomerase, and so a microorganism may be detected by detecting specific single enzymatic products mediated, by topoisomerase I. Generally, type I topoisomerases act by introducing single strand cuts in DNA followed by subsequent ligation of the generated nick in a reaction that involves the formation of a covalent enzyme-DNA cleavage intermediate. Hence, the microorganism can be identified by detecting a nucleic acid substrate which is targeted by a type I topoisomerase of that microorganism, as described further below.

By a type I topoisomerase, we include the meaning of any topoisomerase that can catalyse reversible breakage and rejoining of one strand of DNA in the absence of any energy-donating cofactor(s), changing the linking number in multiples of one (Bhaduir & Nagaraja, 1994 Ind J Biochem Biophys 31: 339-343). Such enzymes differ from type II topoisomerases, which require ATP and catalyse the formulation of transient double-stranded breaks, changing the linking number in steps of two. Type I topoisomerases are subdivided into two main subclasses: type IA topoisomerases which share many structural and mechanistic features with the type II topoisomerases, and type IB topoisomerases, which utilise a controlled rotary mechanism. Examples of type IA topoisomerases include topo I and topo III. Historically, type IB topoisomerases were referred to as eukaryotic topo I, but type IB topoisomerases are present in all three domains of life. Type IA topoisomerases form a covalent intermediate with the 5′ end of DNA and change the linking unit of a circular DNA strand by units of, strictly, one, while type IB topoisomerases form a covalent intermediate with the 3′ end of DNA and change the linking number by multiples of 1 (n). Recently, a type IC topoisomerase has been identified, called topo V, which although structurally unique from type IA and IB topoisomerases, shares a similar mechanism with type IB topoisomerase. For the avoidance of doubt, any type IA, IB and IC topoisomerase is included in the scope of the present invention.

Examples of particular type I topoisomerases that may be detected by the methods of the present invention include a type I topoisomerase from a Mycobacterium (e.g. Mycobacterium tuberculosis (mtTopol) or Mycobacterium smegmatis (msTopol)), and a type I topoisomerase from a Plasmodium (e.g. Plasmodium falciparum (pfTopol)), the amino acid and polynucleotide sequences of which are provided in the list of sequences below (SEQ ID Nos: 18-21). Preferably, the type I topoisomerase from a Mycobacterium is a type IA topoisomerase. Preferably, the type I topoisomerase from a Plasmodium is a type IB topoisomerase. However, other type IA and type IB topoisomerases are also included in the scope of the invention. The type I topoisomerase that may be detected by the methods of the present invention may also include a type I topoisomerase from a Streptococcus such as Group B Streptococcus (GBS), for example Streptococcus agalactiae. The amino acid sequence of the type I topoisomerase from Streptococcus agalactiae is provided below (SEQ ID No: 27; WP_000246605.1)

In another preferred embodiment, the nucleic acid-modifying enzyme is an integrase, and so a microorganism may be detected by detecting specific single enzymatic products mediated by an integrase. Typically, the integrase is a retroviral integrase (e.g. an HIV integrase, SIV integrase, MLV integrase, or FIV integrase). In this embodiment, the method can be used to identify the presence of any pro- and eukaryotic integrating viruses, such as retroviruses, for example HIV, SIV, MLV and FIV.

For the avoidance of doubt, although the methods of the invention described herein are generally used to detect the activity of a single nucleic acid-modifying enzyme, it is also envisaged that they may be used to detect the activity of a nucleic acid-modifying enzyme system. For example, cascades of enzymes can operate to modify a nucleic acid target, and so the detection of such a cascade or system is also included in the scope of the present invention.

By nucleic acid substrate targeted by the nucleic acid-modifying enzyme, we include the meaning of a nucleic acid whose chemical or physical characteristics (e.g. its covalent structure) is altered as a result of the activity of the enzyme. For example, where the enzyme is a type I topoisomerase, the nucleic acid substrate is one that is processed by type I topoisomerase, and when the enzyme is an integrase, the nucleic acid substrate is one that is processed by integrase, and soon. Processing of substrates by enzymes can be determined using an appropriate enzyme assay, for example one which measures depletion of substrate or one which measures accumulation of enzyme product. Enzyme assays for a range of nucleic acid-modifying enzymes are known in the art and are available in kit form.

Preferably, the nucleic acid substrate is one that is selectively targeted by the nucleic acid-modifying enzyme in question. For example, it is preferred if the nucleic acid substrate is targeted by the nucleic acid-modifying enzyme more than it is targeted by any other enzyme (e.g. another enzyme that may be present in the sample), for instance at least 50 times or 100 times or 1000 times more than it is targeted by any other enzyme. Most preferably, the nucleic acid substrate is solely targeted by the nucleic acid substrate. In this way, the nucleic acid substrate is selectively modified by the nucleic acid-modifying enzyme. Similarly, it is preferred if the nucleic acid-modifying enzyme acts on the nucleic acid substrate more than it acts on any other nucleic acid, for instance at least 50 times or 100 times or 100 times more than it acts of any other nucleic acid. Most preferably, the nucleic acid-modifying enzyme solely acts on the nucleic acid substrate.

Thus, it is appreciated that the nucleic acid substrate, is one that is predominantly targeted by the nucleic acid-modifying enzyme of the microorganism which is to be identified, and to a lesser extent by any nucleic acid-modifying enzyme that is native to the sample. By ‘native’ we include the meaning of a natural nucleic acid-modifying enzyme that is expressed by the cells of the subject which the sample originated from, e.g. human cells if the sample originates from a human being, and bovine cells if the sample originates from a bovine subject. Hence, a type I topoisomerase native to a human sample is a human type I topoisomerase and a type I topoisomerase native to a bovine sample is a bovine type I topoisomerase. If, for example, the nucleic acid substrate is intended to detect a Plasmodium type I topoisomerase, the nucleic acid substrate is preferably one that is targeted by a Plasmodium type l topoisomerase to a greater extent than it is targeted by any other type I topoisomerase that may be present in the sample being tested (e.g. an animal or human type I topoisomerase), and so on.

The sequence and structure of appropriate nucleic acid substrates can be designed with respect to the nucleic acid-modifying enzyme for which it is desired to detect. For example, different types of nucleic acid-modifying enzymes are known to have different consensus nucleic acid sequences and/or structural motifs to which they bind, and specific target sequences and structural motifs are known to be targeted with higher efficiency by enzymes of certain organisms than others. In this way, topoisomerases (or other nucleic acid-modifying enzymes) of microorganisms, such as pathogenic and/or parasitic microorganisms, can be distinguished from human and non-human mammal topoisomerase (or other nucleic acid-modifying enzymes). For a given nucleic acid-modifying enzyme, the skilled person will be able to identify an appropriate substrate using established methods in the art. For example, substrates can be generated using standard genetic engineering or synthetic synthesis techniques, and the activity of a particular nucleic acid-modifying enzyme against that substrate assessed by performing an appropriate assay for the activity of the enzyme in question. Typically, enzyme assays measure the level of product generated by the enzymatic reaction, which level is often conveniently indicated by a detectable signal. Also, research has been done on determining both the structural and sequence specificity of nucleic acid-modifying enzymes, and so one can consult the scientific literature to facilitate the design of an appropriate substrate.

In addition to the sequence and structure of the nucleic acid substrate, it will also be understood that the specificity of enzyme action on the nucleic acid substrate can be managed by controlling the conditions under which the nucleic acid substrate is brought into contact with the sample. Different enzymes operate under different conditions and so it may be desirable to manipulate the conditions of the contact so that they favour action by the nucleic acid-modifying enzyme that is to be detected and/or disfavour action by any other nucleic acid-modifying enzyme.

The nucleic acid substrate may be single stranded or double stranded nucleic acid. When the nucleic acid substrate is acted on in a double stranded form, it will be understood that the nucleic acid substrate may be provided as two single molecules, which are hybridised, or as a single nucleic acid which folds into a secondary hairpin structure comprising a double-stranded target region. In the former case, one of the molecules may also correspond to the further nucleic acid molecule as is described further below. The target region may comprise the sequence and/or structure that confers the specificity between the enzyme and the nucleic acid substrate. It may be desirable to label the nucleic acid substrate with a detectable moiety.

The nucleic acid substrate may be between 5 and 500 nucleotides in length. More specifically, the nucleic acid substrate may be between 5 and 400 nucleotides in length, such as between 5 and 300, 5 and 200, 5 and 100, or 5 and 50 nucleotides in length. Typically, the nucleic acid substrate is between 20 and 50 nucleotides in length, such as between 25 and 45 nucleotides in length. Thus, the nucleic acid substrate may be 26, 27, 28, 29, 30, 31, 32, 33, 34, 35, 36, 37, 38, 39, 40, 41, 42, 43, or 44 nucleotides in length.

In one embodiment, the nucleic acid-modifying enzyme is a type I topoisomerase from the Plasmodium genus, the microorganism is a Plasmodium, and the nucleic acid substrate is one that is selectively targeted by a Plasmodium type I topoisomerase. For example, the nucleic acid substrate is targeted by a Plasmodium type I topoisomerase more than it is targeted by any other type I topoisomerase from another source (e.g. an animal or human type I topoisomerase), for instance at least 50 times or 100 times or 100,times more. Most preferably, the nucleic acid substrate is solely targeted by a Plasmodium type I topoisomerase.

In a specific embodiment when the nucleic acid-modifying enzyme is a type I topoisomerase from the Plasmodium genus, the nucleic acid substrate is double stranded DNA wherein at least one strand comprises a sequence selected from the group consisting of ACTACCATTCTGAGTCGTTCGAAGTTCCTATACTTT (SEQ ID No: 1) and TCTAGAAAGTATAGGAACTTCGAACGACTCAGAATG (SEQ ID No: 2); a sequence sharing at least 30% sequence identity thereto, such as at least 40%, 50%, 60%, 70%, 75%, 80%, 85%, 90%, 95% or 99% sequence identity thereto; or a sequence which is a part of at least 5 consecutive nucleotides, such as at least 10, 15, 20, 25 or 30 consecutive nucleotides, of any of said sequences. In this case, the microorganism is selected from the Plasmodium genus, for example the microorganism is Plasmodium falciparum. Preferably, the nucleic acid substrate is double stranded DNA wherein the first strand comprises the sequence ACTACCATTCTGAGTCGTTCGAAGTTCCTATACTTT (SEQ ID No: 1) and the second strand comprises the sequence TCTAGAAAGTATAGGAACTTCGAACGACTCAGAATG (SEQ ID No: 2). To the extent that the nucleic acid substrate varies from the sequences of SEQ ID Nos: 1 and 2, it will be appreciated that the polynucleotides must still serve as substrates for the type I topoisomerase from the Plasmodium genus.

In another specific embodiment when the nucleic acid-modifying enzyme is a type I topoisomerase from the Plasmodium genus, the nucleic acid substrate is double stranded DNA wherein at least one strand comprises a sequence selected from the group consisting of ACTACCATTCTGAGTCGTTCGATCTAAAAGACTTAGA (SEQ ID No: 3) and CTAAGTCTTTTAGATCGAACGACTCAGAATG (SEQ. ID No: 4); a sequence having at least 30% sequence identity thereto, such as at least 40%, 50%, 60%, 70%, 75%, 80%, 85%, 90%, 95% or 99% sequence identity thereto; or, a sequence which is a part of at least 5 consecutive nucleotides, such as at least 10, 15, 20, 25, 30 or 35 consecutive nucleotides, of any of said sequences. In this case, the microorganism is selected from the Plasmodium genus, for example the microorganism is Plasmodium falciparum. Preferably, the nucleic acid substrate is double stranded DNA wherein the first strand comprises the sequence ACTACCATTCTGAGTCGTTCGATCTAAAAGACTTAGA (SEQ ID No: 3) and the second strand comprises the sequence ATTTTTCTAAGTCTTTTAGATCGAACGACTCAGAATG (SEQ ID No: 4). To the extent that the nucleic acid substrate varies from the sequences of SEQ ID Nos: 3-and 4, it will be appreciated that the polynucleotides must still serve as substrates for the type I topoisomerase from the Plasmodium genus.

It will be appreciated that the sequence of the nucleic acid substrate is only one parameter that is important in conferring specificity towards type I topoisomerases, and so nucleic acid substrates may comprise sequences different to those above but nevertheless share the same specificity for a given type I topoisomerase. For example, nucleic acid substrates having a cleavage site near a protruding 5′ end are believed to be the main feature of nucleic acid substrates that are targeted by Plasmodium type I topoisomerases (human type I topoisomerase is not able to cleave close to the end of this substrate). The skilled person can readily design and test appropriate substrates using standard techniques in the art. Also, the skilled person can make use of what is known about the nucleic acid substrates of well characterised type I topoisomerases to design candidate nucleic acid substrates for those type I topoisomerases that are less well characterised (see, for example, Perry and Mondragon, 2003, Structure 11(11): 1349 which describes the structure of a complex between E. coli DNA topoisomerase I and single-stranded DNA).

In one embodiment, the nucleic acid-modifying enzyme is a typed topoisomerase from the Mycobacterium genus, the microorganism is a Mycobacterium (e.g. Mycobacterium tuberculosis), and the nucleic acid substrate is one that is selectively targeted by a Mycobacterium type I topoisomerase. For example, the nucleic acid substrate is targeted by a Mycobacterium type I topoisomerase more than it is targeted by any other type I topoisomerase from another source (e.g. an animal or human type I topoisomerase), for instance at least 50 times or 100 times or 100 times more. Most preferably, the nucleic acid substrate is solely targeted by a Mycobacterium type I topoisomerase.

In a specific embodiment when the nucleic acid-modifying enzyme is a type I topoisomerase from the Mycobacterium genus, the nucleic acid substrate is single stranded DNA that comprises the sequence CAGTGAGCGAGCTTCCGCTTGACATCCCAATAGTTTCTCTTC (SEQ ID No: 5); a sequence sharing at least 30% sequence identity thereto, such as at least 40%, 50%, 60%, 70%, 75%, 80%, 85%, 90%, 95% or 99% sequence identity thereto; or a sequence which is a part of at least 5 consecutive nucleotides, such as at least 10, 15, 20,25 or 30 consecutive nucleotides, of said sequence. In this case, the microorganism is selected from the Mycobacterium genus, for example the microorganism is Mycobacterium tuberculosis. Preferably, the nucleic acid substrate is single stranded DNA that comprises the sequence CAGTGAGCGAGCTTCCGCTTGACATCCCAATAGTTTCTCTTC (SEQ ID No: 5). To the extent that the nucleic acid substrate varies from the sequence of SEQ ID No: 5, it will be appreciated that the polynucleotide must still serve as substrate for the type I topoisomerase from the Mycobacterium genus.

In one embodiment, the nucleic acid-modifying enzyme is a type I topoisomerase from the Streptococcus genus (e.g. GBS, such as Streptococcus agalactiae), the microorganism is a Streptococcus (e.g. GBS, such as Streptococcus agalactiae), and the nucleic acid substrate is one that is selectively targeted by a Streptococcus type I topoisomerase (e.g. GBS, such as Streptococcus agalactiae). For example, the nucleic acid substrate may be targeted by a Streptococcus (e.g. GBS, such as Streptococcus agalactiae) type I topoisomerase more than it is targeted by any other type I topoisomerase from another source (e.g. an animal or human type I topoisomerase), for instance at least 50 times or 100 times or 100 times more. Most preferably, the nucleic acid substrate is solely targeted by a Streptococcus type I topoisomerase (e.g. GBS, such as Streptococcus agalactiae).

In a specific embodiment when the nucleic acid-modifying enzyme is a type I topoisomerase from the Streptococcus genus (e.g. GBS, such as Streptococcus agalactiae), the nucleic acid substrate is single stranded DNA that comprises the sequence ACTTCGGGATGCTGAAACGCAGCTGTGC (SEQ ID No: 28); a sequence sharing at least 30% sequence identity thereto, such as at least 40%, 50%, 60%, 70%, 75%, 80%, 85%, 90%, 95% or 99% sequence identity thereto; or a sequence which is a part of at least 5 consecutive nucleotides, such as at least 10, 15, 20 or 25 consecutive nucleotides, of said sequence. In this case, the microorganism is selected from the Streptococcus genus, for example the microorganism is Group B Streptococcus such as Streptococcus agalactiae. Preferably, the nucleic acid substrate is single stranded DNA that comprises the sequence ACTTCGGGATGCTGAAACGCAGCTGTGC (SEQ ID No: 28). To the extent that the nucleic acid substrate varies from the sequence of SEQ ID No: 28, it will be appreciated that the polynucleotide must still serve as substrate for the type I topoisomerase from the Streptococcus genus. The topoisomerase is expected to cleave at the site indicated by the arrow: ACTTCGGG↓ATGCTGAAACGCAGCTGTGC (SEQ ID No: 28). Upon cleavage, the topoisomerase will be covalently attached to the 3′ end and will be able to ligate to a further nucleic acid molecule carrying a free 3′ OH group, as described further below.

In an embodiment when the nucleic acid-modifying enzyme is an integrase, the nucleic acid substrate is a long terminal repeat (LTR) sequence that is selectively targeted by the integrase. In this case, the microorganism is a virus, such as a retrovirus. Considerable research has been done on elucidating LTR sequences, and so published literature on LTR sequences can be searched to find the appropriate sequence for a given integrase.

An example of an appropriate nucleic acid substrate to detect HIV integrase is the double stranded DNA wherein the first strand comprises the sequence TTTAGTCAGTGTGGAAAATCTCTAGCAGT (SEQ ID No: 6) and the second strand comprises the sequence ACTGCTAGAGATTTTCCACACTGACTAAA (SEQ ID No: 7). Hence in a specific embodiment, the nucleic acid substrate is double stranded DNA wherein at least one strand comprises a sequence selected from the group consisting of TTTAGTCAGTGTGGAAAATCTCTAGCAGT (SEQ ID No: 6) and ACTGCTAGAGATTTTCCACACTGACTAAA (SEQ ID No: 7); a′sequence sharing at least 30% sequence identity thereto, such as at least 40%, 50%, 60%, 70%, 75%, 80%, 85%, 90%, 95% or 99% sequence identity thereto; or a sequence which is a part of at least 5 consecutive nucleotides of any of said, such as at least 10, 15, 20 or 25 consecutive nucleotides. The key specificity determining feature of SEQ ID No: 6 is the four 3′ end nucleotides (CAGT; SEQ ID No: 8): ‘CA’ is conserved across all retroviruses, whereas the two terminal nucleotides may vary between retroviruses. Thus, to the extent that the nucleic acid substrate is different from SEQ ID No: 6, it is preferred that the ‘CA’ dinucleotide remains intact, and even more preferred that the four 3′ end nucleotides (CAGT; SEQ ID No: 8) remain intact. However, it will be appreciated that by varying the two 3′ end nucleotides, one may change the specificity of the substrate for a different integrase (i.e. one other than HIV integrase) if desired. Preferably, the nucleic acid substrate is the double stranded DNA wherein the first strand comprises the sequence TTTAGTCAGTGTGGAAAATCTCTAGCAGT (SEQ ID No: 6) and the second strand comprises the sequence ACTGCTAGAGATTTTCCACACTGACTAAA (SEQ ID No: 7). To the extent that the nucleic acid substrate varies from the sequences of SEQ ID Nos: 6 and 7, it will be appreciated that the polynucleotides must still serve as substrates for the HIV integrase.

An example of an appropriate nucleic acid substrate to detect MLV integrase is the double stranded DNA wherein the first strand comprises the sequence TTGACTACCCGTCAGCGGGGGTCTTTCATT (SEQ ID No: 22) and the second strand comprises the sequence AATGAAAGACCCCCGCTGACGGGTAGTCAA (SEQ ID No: 23). Hence in a specific embodiment, the nucleic acid substrate is double stranded DNA wherein at least one strand comprises a sequence selected from the group consisting of TTGACTACCCGTCAGCGGGGGTCTTTCATT (SEQ ID No: 22) and AATGAAAGACCCCCGCTGACGGGTAGTCAA (SEQ ID No: 23); a sequence sharing at least 30% sequence identity thereto, such as at least 40%, 50%, 60%, 70%, 75%, 80%, 85%, 90%, 95% or 99% sequence identity thereto; or a sequence which is a part of at least 5 consecutive nucleotides of any of said, such as at least 10, 15, 20 or 25 consecutive nucleotides. The key specificity determining feature of SEQ ID No: 22 is the four 3′ end nucleotides (CATT; SEQ ID No: 24): ‘CA’ is conserved across all retroviruses, whereas the two terminal nucleotides may vary between retroviruses. Thus, to the extent that the nucleic acid substrate is different from SEQ ID No: 22, it is preferred that the ‘CA’ dinucleotide remains intact, and even more preferred that the four 3′ end nucleotides (CATT; SEQ ID No: 24) remain intact. However, it will be appreciated that by varying the two 3′ end nucleotides, one may change the specificity of the substrate for a different integase (i.e. one other than MLV integrase) if desired. Preferably, the nucleic acid substrate is the double stranded DNA wherein the first strand comprises the sequence TTGACTACCCGTCAGCGGGGGTCTTTCATT (SEQ ID No: 22) and the second strand comprises the sequence AATGAAAGACCCCCGCTGACGGGTAGTCAA (SEQ ID No: 23). To the extent that the nucleic acid substrate varies from the sequences of SEQ ID Nos: 22 and 23, it will be appreciated that the polynucleotides must still serve as substrates for the MLV integrase.

By the term “sequence identity”, we include the meaning of a quantitative measure of the degree of homology between two amino acid sequences or between two nucleic acid sequences of equal length. If the two sequences to be compared are not of equal length, they must be aligned to give the best possible fit, allowing the insertion of gaps or, alternatively, truncation at the ends of the polypeptide sequences or nucleotide sequences. The sequence identity can be calculated, wherein Ndif is the total number of non-identical residues in the two sequences when aligned and wherein Nref is the number of residues in one of the sequences, preferably sequence identity is calculated over the full length reference as provided herein. Hence, the DNA sequence AGTCAGTC (SEQ ID No: 9) will have a sequence identity of 75% with the sequence AATCAATC (SEQ ID No: 10) (Ndif=2 and Nref=8). A gap is counted as non-identity of the specific residue(s), i.e. the DNA sequence AGTGTC (SEQ ID No: 11) will have a sequence identity of 75% with the DNA sequence AGTCAGTC (SEQ ID No: 9) (Ndif=2 and Nref=8).

With respect to all embodiments of the invention relating to nucleotide sequences, the percentage of sequence identity between one or more sequences may also be based on alignments using the clustalW software (http:/www.ebi.ac.uk/clustalW/index.html) with default settings. For nucleotide sequence alignments these settings are:

Alignment=3Dfull, Gap Open 10.00, Gap Ext. 0.20, Gap separation Dist. 4, DNA weight matrix: identity (IUB).

The sample may be any suitable biological or non-biological sample, and may originate, be obtained or be isolated from any source that is of interest for detection of specific microorganisms. The choice of sample depends on the microorganism, disease or infectious disorder to be determined, as well as the detection method and will be appreciated by those skilled in the art.

An example of a non-biological sample is water, such as drinking water or domestic water, which may be subjected to analysis for detection of contamination with microorganisms, such as infectious agents, for example pathogenic bacteria or parasitic microorganisms (e.g. Mycobacteria or Plasmodium). However, any other non-biological sample may be assessed where it is desirable to determine whether that sample contains a particular microorganism. For example, the sample may be obtained from any source of human or animal consumption, such as food or feed, i.e. the sample may be a food or feed sample. Alternatively, samples may be prepared from facilities used in food production such as conveyor belts.

Biological samples may originate, be obtained or isolated from any subject of the animal kingdom, depending upon the intended use of the method of the invention. For example, the sample may originate, be obtained or isolated from any subject of vertebrates, such as mammals, reptiles, fish, birds, and amphibians. Preferably, the biological sample is isolated or originating or obtained from a mammalian subject, such as a human being or a bovine subject, i.e. it is a human or bovine sample. In other examples, the sample is a sample originating, obtained or isolated from a ruminant, a ferret, a badger, a rodent, an elephant, a sheep, a goat, a duck, a chicken, a turkey, a koala, a bird, a pig, a deer, a coyote, a camel, a puma, a fish, a dog, a cat, a non-human primate, or a non-human animal.

When the sample of the invention is a biological sample, the sample typically comprises cells that originate from the subject from which the sample is isolated. Thus, in one embodiment, the sample comprises eukaryotic cells, such as mammalian (e.g. human or bovine), reptile, fish, bird or amphibian cells. The cells in the sample may be lysed prior to the sample being brought into contact with the nucleic acid substrate, for example by placing in a hypotonic buffer, or by sonication or by microfluidic technology.

In one embodiment, the sample is a blood sample, a tissue sample, or a secretion sample. Conveniently, the sample is a blood sample. By ‘blood sample’ we include whole blood and any fraction of blood, such as blood plasma or blood serum. The tissue sample may be any sample of a tissue selected from the group consisting of skin, epidermis, dermis, hypodermis, breast, fat, thymus, gut, small intestine, large intestine, stomach, muscle, pancreas, heart muscle, skeletal muscle, smooth muscle, liver, lung, brain, cornea and tumours, ovarian tissue, uterine tissue, colon tissue, prostate tissue, lung tissue, renal tissue, thymus tissue, testis tissue, hematopoietic tissue, bone marrow, urogenital tissue, expiration air, stem cells, including cancer stem cells, biopsies, and cerebrospinal fluid. Further examples of possible samples include semen, sputum, saliva, ova, hairs, nails, tears, urine, cerebrospinal fluid, a cell smear, a biopsy and faeces. Yet further possible samples include vaginal or rectal swabs.

As mentioned above, according to the method of the first aspect of the invention, a microorganism is identified in a sample by detecting the activity of a nucleic acid-modifying enzyme of that microorganism via its effect on a nucleic acid substrate.

The first step of the method involves contacting the sample with the nucleic acid substrate. The conditions in which the sample is brought into contact with the nucleic acid substrate should support binding of a nucleic acid-modifying enzyme within the sample to the nucleic acid substrate. Conveniently, the conditions are also conducive to the activity of the nucleic acid modifying enzyme, and ideally are set to match the optimum temperature and pH of the nucleic acid-modifying enzyme in question. For example, the nucleic acid substrate may be brought into contact with the sample at a temperature of around 20-40° C., for example 25-40° C. or 30-40° C., such as around 37° C., and/or at a pH of between 7.0 and 7.5. Typical incubation times for the contact step range from 1 minute to 2 hours, such as between 5 and 100 minutes, 10 and 60 minutes, or 20 and 30 minutes.

In a preferred embodiment of the first aspect of the invention, the nucleic acid substrate is immobilised on a surface, and prior to step (b), the nucleic acid substrate on the surface is washed so as to remove substantially all non-specifically bound material from the substrate. Thus, it will be appreciated that the invention includes a method of identifying a microorganism that expresses a nucleic acid-modifying enzyme, in a sample, the method comprising:
(a)(i) contacting a nucleic acid substrate targeted by the nucleic acid-modifying enzyme with the sample, wherein the nucleic acid substrate is immobilised on a surface;
(a)(ii) washing the nucleic acid substrate immobilised on the surface so as to remove substantially all non-specifically bound material from the substrate;
(b) adding a further nucleic acid molecule to the sample which nucleic acid molecule is ligated to the nucleic acid substrate in the presence of the nucleic acid-modifying enzyme to form a ligation product which comprises a linear single strand of nucleic acid that is capable of being detected; and
(c) detecting the presence of the ligation product.

Preferably, the nucleic acid substrate is immobilised on to a solid support, typically one that is non-porous. Examples of suitable supports include glass, paper, cardboard, sepharose, agarose, plastic, metal, silicon, ceramics and latex. The nucleic acid substrate may be attached directly to the surface or it may be attached indirectly, for example via one or more linking moieties.

Generally, the surface and/or nucleic acid substrate are modified so as to facilitate immobilisation of the nucleic acid substrate onto the surface. For example, functional groups that are reactive with the nucleic acid substrate, or that are reactive with a linker moiety that in turn binds to, or reacts with, the nucleic acid substrate, may be introduced into the surface or used to coat the surface. Similarly, the nucleic acid substrate may be modified with functional groups in order to have attachment to a reactive group on the surface or linker moiety. Common surface modifications include amino groups, halogen groups, epoxy groups, activated carboxy groups and the like. Typical nucleic acid modifications comprise amino groups, succinyl groups, thiol groups, hydrazide groups and the like. Methods of derivatising or functionalising surfaces in this way are well known in the art, and the skilled person is able to select the appropriate derivatisation or functionalisation depending on the surface (see, for example, Hermanson, “Immobilised Affinity Ligand Techniques”, Academic Press, Inc, San Diego Calif. (1992); Klein, “Affinity Membranes: Their Chemistry and Performance in Adsorptive Separation Processes”, J Wiley and Sons, NY (1991). Methods for immobilising nucleic acids onto solid supports are also described in Du et al (Top Curr Chem 2006, 261: 45-61), and any such method may be used.

Any of the conventional ways of cross-linking molecules, such as those generally described in O'Sullivan et al Anal. Biochem. (1979) 100, 100-108 may be used to immobilise the nucleic acid substrate on to the surface (e.g. solid support). It will be understood that a large number of homobifunctional and heterobifunctional crosslinking chemistries would be appropriate to join the surface (e.g. solid support) with the nucleic acid substrate, and any such chemistry may be used. For example, Click Chemistry using Staudinger Ligation Chemistry (phosphine-azido chemistry) may be used. In another example, an amino-modified nucleic acid substrate may be reacted with a bifunctional agent capable of reacting with those amino groups, for example through binding to a N-hydroxysuccinimide ester.

In a preferred embodiment, the nucleic acid substrate is modified with an amine group, and the solid support is one that is capable of reacting with such an amine group. For instance, the solid support may be one that is coated with maleic anhydride (e.g. Well-Coated™ Amine Binding 8-well strip plates from G-biosciences).

Conveniently, the nucleic acid substrate is fixed to the surface (e.g. solid support) by covalent bonding. However, it will be appreciated that non-covalent interactions (e.g. based on avidin/streptavidin/biotin or antibody/antigen interactions) may also be used. For example, the surface may be biotinylated and coated with streptavidin, which in turn binds to a biotinylated nucleic acid substrate.

Another suitable means for immobilising the nucleic acid substrate to the surface (e.g. solid support) is via gold/sulpho-nucleic acid binding.

The surface (e.g. solid support) to, which the nucleic acid substrate is immobilised can take any form, and may be a two-dimensional surface or a three-dimensional surface. For example, the surface may be a planar surface such as a slide (e.g. glass microscope slide) or chip (e.g. silicon chip), or it may be a bead or particle or micro-sphere (e.g. polystyrene, magnetic or silica micro-spheres) or other geometric shape or configuration known in the art. When the surface is in the form of a bead or particle or micro-sphere, it will be understood that it may constitute the matrix of a column.

The surface can be washed by any liquid which does not disrupt the interaction between the nucleic acid substrate and surface. Suitable buffers are described in the Examples and include Tris based buffers and saline sodium citrate buffers.

By removing substantially all non-specifically bound material from the substrate, we include the meaning of removing substantially all material other than the nucleic acid-modifying enzyme from the substrate. For example, the sample may contain agents that bind to the substrate non-specifically, but that nevertheless compete with the nucleic acid-modifying enzyme, or which otherwise reduce, binding of the nucleic acid-modifying enzyme, for the nucleic acid substrate. Such agents include other enzymes, which may be other nucleic acid-modifying enzymes, and nucleic acid. Removing non-specifically bound material in this way is expected to increase the specificity of the method.

Preferably, the immobilised surface is washed so that at least about 50% of such material is removed, preferably at least about 60%, and more preferably at least about 70%, about 80%, about 90%, about 95% and about 99% of such material is removed. Most preferably, the immobilised surface is washed so that only the nucleic acid-modifying enzyme is attached to the nucleic acid substrate. This can be assessed by analysing what is bound to the nucleic acid substrate after contact with the sample, for example, by electrophoretic mobility shift assays. By comparing the analysis with that of a control nucleic acid substrate contacted with only the nucleic acid-modifying enzyme, one can examine whether the intensity of the washing needs to be modified (e.g. by altering strength of buffer) and/or whether the washing process needs to be repeated. Typically, the surface is washed at least one time, two times or three times.

Detection of the activity of the nucleic acid-modifying enzyme relies upon the enzyme catalysed formation of a ligation product between the nucleic acid substrate and a further nucleic acid molecule. Thus, as well as bringing the nucleic acid substrate into contact with the sample, it must also be brought into contact with the further nucleic acid molecule. The further nucleic acid molecule may be, brought into contact with the nucleic acid substrate before, at the same time as, or after, the sample. Preferably, the further nucleic acid molecule is brought into contact with the nucleic acid substrate after the sample, and most preferably after the nucleic acid substrate has been washed as explained above. However, it may be desirable for the further nucleic acid substrate to be brought into contact with the nucleic acid substrate before the sample. In this case, both the nucleic acid substrate and the further nucleic acid molecule may be immobilised to the surface, which provides a useful format for various applications of the method of the invention (e.g. the reagents used in the method can be provided as stick tests or dipsticks). As is explained further below, the further nucleic acid molecule may be a part of the nucleic acid substrate in which case the nucleic acid substrate and further'nucleic acid molecule are clearly in contact with each other, before being exposed to the sample.

The nucleic acid substrate and further nucleic acid molecule are brought into contact with each other under conditions that support their ligation to one another in the presence of the nucleic acid-modifying enzyme. Such conditions will depend upon the nucleic acid-modifying enzyme in question, and are ideally set to match the optimum temperature and pH of that enzyme. The conditions can be readily identified by the skilled person, for example by consulting scientific literature. Examples of such conditions for type I topoisomerase and integrase are also provided in the Examples. Typically, the nucleic acid substrate, further nucleic acid molecule and sample are brought into contact with the sample at a temperature of around 20-40° C., for example 25-40° C. or 30-40° C., such as around 37° C., and/or at a pH of between 7.0 and 7.5. The reaction mixture is incubated for a sufficient length of time to allow for ligation of the nucleic acid substrate to the further nucleic acid molecule. Typical incubations times range from 1 minute to 2 hours, such as between 5 and 100 minutes, 10 and 60 minutes, or 20 and 30 minutes.

In addition to washing the nucleic acid substrate immobilised on the surface, another way of minimising false positives is by depleting the sample and/or reaction buffers of entities that are required for the activity of other nucleic acid-modifying enzymes that may be present in the sample but which are not required for the, activity of the nucleic acid-modifying enzyme for which it is desired to detect. For example, the sample or reaction buffers may be depleted of divalent cations, which are a prerequisite for the activity of most nucleic acid-modifying enzymes, including ligases, but not for type I topoisomerases such as Plasmodium falciparum type I topoisomerase and Mycobacterium tuberculosis type I topoisomerase. Thus, in an embodiment, the sample and/or reaction buffers are depleted of divalent cations. This may be done, for example, by adding an agent for depletion of divalent cations to the sample prior to its combination with the nucleic acid substrate, or the substrate is mixed with such an agent for depletion of divalent cations in order to reduce the activity of other nucleic acid-modifying enzymes which are not intended to be detected. The agent for depletion of divalent cations include a chelating agent, such as EDTA, or any other effective agent.

The further nucleic acid molecule may be any nucleic acid molecule as defined herein provided that it can be ligated to the nucleic acid substrate in the presence of the nucleic acid-modifying enzyme to form a ligation product that comprises a linear single stand of nucleic acid that is capable of being detected. Typically, the further nucleic acid molecule is double stranded DNA or single stranded DNA. As explained further below, the further nucleic acid molecule may be a part of the nucleic acid substrate. For example, it may comprise one strand of a double stranded nucleic acid substrate. It will be appreciated that the linear single strand of nucleic acid that is capable of being detected is not present, or else is not capable of being detected, prior to ligation of the nucleic acid substrate and further nucleic acid molecule. Thus, the ligation product may comprise a new polynucleotide sequence that can be detected, which sequence was not present, or was not detectable, prior to ligation. In this way, detection of the linear single strand of nucleic acid can be used as a direct readout of the formation of the ligation product and hence the presence of the nucleic acid-modifying enzyme.

Pairs of nucleic acid substrates and further nucleic acid molecules can be readily designed so as to ensure that the ligation product they form when they are ligated to each other contains the linear single strand of nucleic acid that is capable of detection. Suitable pairs are described herein and in the Examples. As an example, when the nucleic acid substrate is double stranded DNA, the ends of the DNA may comprise an overhang, and ligation of a single stranded further nucleic acid molecule to one of the ends can create a ligation product that contains a protruding single strand of nucleic acid that can be detected. Alternatively, a linear single stranded nucleic acid capable of detection may be formed via asymmetric cleavage of the nucleic acid substrate by the nucleic acid-modifying enzyme. Alternatively, the linear single stranded nucleic acid capable of detection may be formed via ligation of two single strands of nucleic acid. Alternatively, the further nucleic acid molecule may a double stranded nucleic acid that comprises an overhang. Further configurations of nucleic acid substrate and further nucleic acid molecule will be apparent to the skilled person in accordance with standard molecular biology techniques, and are shown in the Examples and Figures. The skilled person will be able to design such pairs of nucleic acid substrate and further nucleic acid molecule, for example by reference to the scientific literature regarding the target sequence of the nucleic acid modifying enzyme in question (e.g. type I topoisomerase), the behaviour of the enzyme in question, and basic principles of oligonucleotide design (such as melting temperature and consequences of combining certain base sequences, PCR methods and vectors).

The further nucleic acid molecule may be part of the nucleic acid substrate. In this embodiment, the nucleic acid substrate is targeted by the nucleic acid-modifying enzyme which ligates one strand of the nucleic acid substrate to another strand of the nucleic acid substrate so as to form a ligation product which comprises a linear single strand of nucleic acid that is capable of being detected. In this embodiment, the nucleic acid substrate is typically double stranded, and the further nucleic acid molecule comprises one of the strands of the nucleic acid substrate but is not ligated to the other strand in the absence of a nucleic acid-modifying enzyme. Thus, the invention provides a method of identifying a microorganism that expresses a nucleic acid-modifying enzyme, in a sample, the method comprising:
(a) contacting a nucleic acid substrate targeted by the nucleic acid-modifying enzyme with the sample, wherein the nucleic acid substrate is capable of being processed in the presence of the nucleic acid modifying enzyme to form a ligation product which comprises a linear single strand of nucleic acid that is capable of being detected; and
(b) detecting the presence of the ligation product.

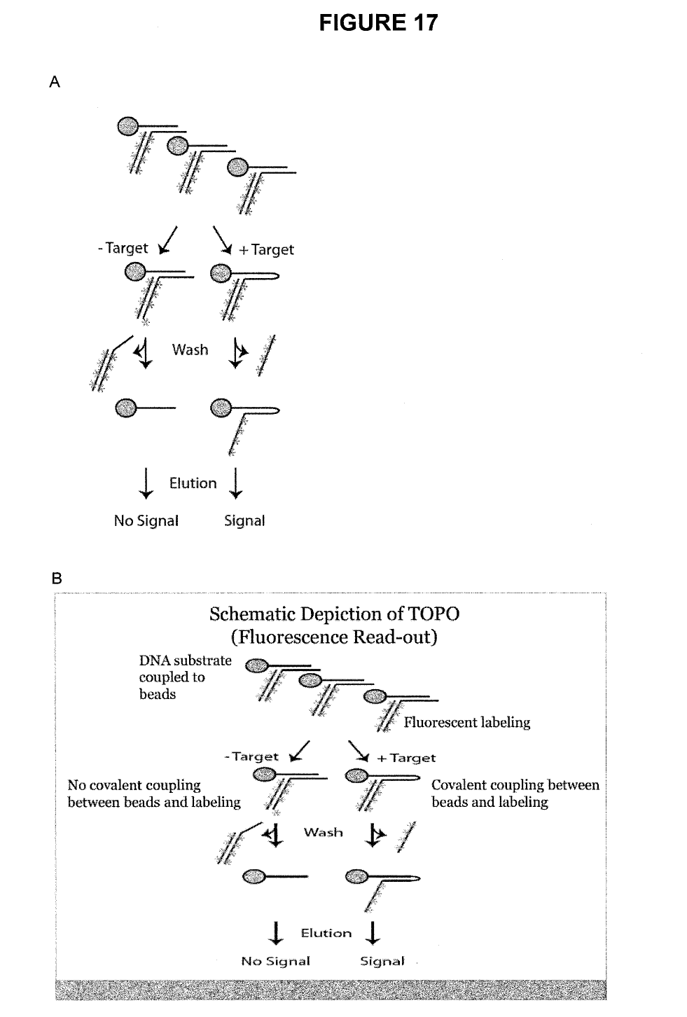

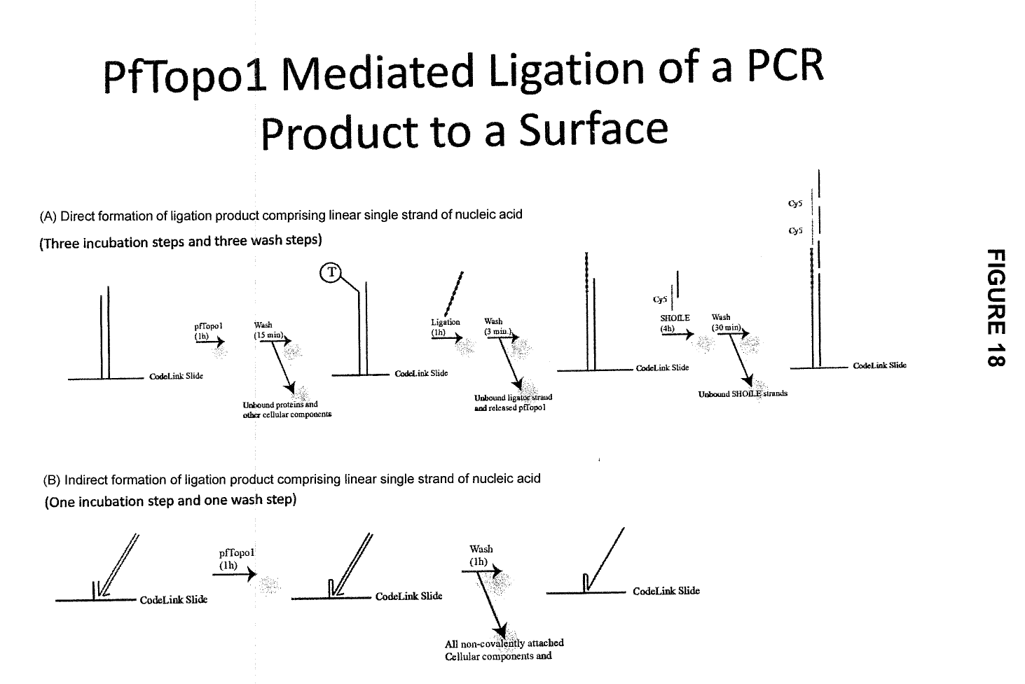

By a nucleic acid substrate that is capable of being processed in the presence of the nucleic acid-modifying enzyme to form a ligation product which comprises a linear single strand of nucleic acid that is capable of being detected, we include the meaning of a nucleic acid substrate that has a first and second strand that can be ligated together in the presence of a nucleic acid-modifying enzyme. In this way, the further nucleic acid molecule may comprise the first or second strand of the nucleic acid substrate. However, it will be appreciated that the further nucleic acid molecule may itself also comprise a double stranded portion that is distinct from the portion of the molecule that makes up the nucleic acid substrate. Where the nucleic acid-modifying enzyme has cleavage activity as well as ligase cleavage, for example in the case of type I topoisomerase, it will be understood that one or more of the first and second strands may be cleaved by the nucleic acid-modifying enzyme prior to being ligated to one another. This embodiment, where the further nucleic acid molecule is part of the nucleic acid substrate, is illustrated in FIGS. 17 and 18(B).

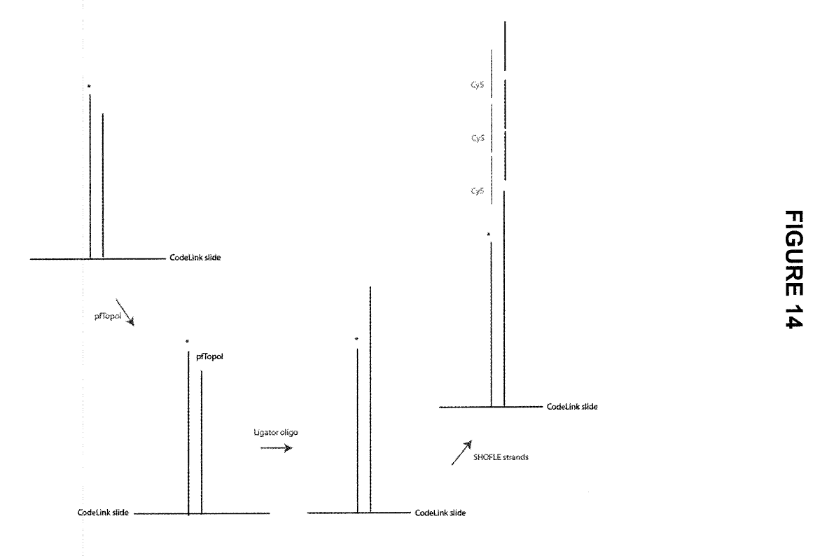

For the avoidance of doubt, it will be appreciated that the further nucleic acid molecule may be ligated to the nucleic acid substrate to form the ligation product (which comprises a linear single strand of nucleic acid that is capable of being detected) either directly or indirectly. When the ligation product is formed directly, the product formed when the nucleic acid substrate and further nucleic acid molecule are ligated, itself, comprises a linear single strand of nucleic acid without the need for further processing steps. Examples of direct formation of the ligation product are shown in FIGS. 14-16 and 18(A). When the ligation product is formed indirectly, the ligation product that comprises a linear single strand of nucleic acid is produced via the formation of one or more intermediate products when the nucleic acid substrate and further nucleic acid molecule are ligated. Thus, the nucleic acid substrate and further nucleic acid molecule may be ligated to form an intermediate product which is then further processed to produce the ligation product that comprises a linear single strand of nucleic acid. The intermediate product may comprise double stranded nucleic acid, and the further processing may involve exposing the intermediate product to denaturing conditions such that strands of double stranded nucleic acid are separated, thereby exposing a linear single strand of nucleic acid. Suitable denaturing conditions include those that disrupt the hydrogen bonding between nucleotide bases in the double stranded nucleic acid. Examples of denaturing conditions are well known in the art and include urea, formamide and heat. Thus, the processing may include washing an intermediate product comprising double stranded nucleic acid in a buffer comprising urea (e.g. 6-8 M urea), for example. An example of such processing is shown in FIGS. 17 and 18(B).

The invention includes a method of identifying a microorganism that expresses a nucleic acid-modifying enzyme, in a sample, the method comprising:
(a) contacting a nucleic acid substrate targeted by the nucleic acid-modifying enzyme with the sample, wherein the nucleic acid substrate is capable of being processed in the presence of the nucleic acid modifying enzyme to form a ligation product which comprises a linear single strand of nucleic acid that is capable of being detected, and wherein the nucleic acid substrate is immobilised on a surface;
(b) washing the nucleic acid substrate immobilised on the surface so as to remove substantially all non-specifically bound material from the substrate; and
(c) detecting the presence of the ligation product.

In this embodiment, the nucleic acid substrate is typically double stranded nucleic acid whereby, in the presence of the nucleic acid-modifying enzyme, a first strand is ligated to a second strand to form a ligation product which comprises a linear single strand of nucleic acid that is capable of being detected. The first strand may be immobilised to the surface and the second strand may be hybridised to the first strand without being immobilised to the surface. Preferably, the surface is a bead or slide. It will be appreciated that the wash step (b) may involve exposing an intermediate product formed by ligation of a first and second strand of the nucleic acid substrate, to denaturing conditions (e.g. urea such as 6-8M urea) such that strands of double stranded nucleic acid are separated, thereby exposing a linear single strand of nucleic acid. This is illustrated in FIGS. 17 and 18(B).

Preferably, in this embodiment, the nucleic acid substrate is double stranded, wherein a first strand is immobilised to a surface (e.g. a bead or slide) and a second strand (corresponding to the further nucleic acid molecule) is hybridised to the first strand without itself being immobilised to the surface. It is also preferred if a portion of the further nucleic acid molecule, separate from the portion that is hybridised to the first strand of the nucleic acid substrate, comprises double stranded nucleic acid. In this case, in the presence of a nucleic acid-modifying enzyme, an intermediate product is formed whereby the first and second strand of the nucleic acid substrate are ligated to each other. Washing of the nucleic acid substrate immobilised to the surface under denaturing conditions then results in the formation of a ligation product that comprises a linear single strand of nucleic acid that is capable of being detected, the linear single strand corresponding to the strand of the portion of the double strand nucleic acid of the further nucleic acid molecule that remains attached to the nucleic acid substrate. In the absence of a nucleic acid-modifying enzyme, no ligation of the first and second strands of the nucleic acid substrate takes place, and so washing of the nucleic acid substrate immobilised to the surface, under denaturing conditions, simply results in the, first strand of the nucleic acid substrate remaining immobilised on the surface. The second strand that is attached to the rest of the further nucleic acid molecule is released.

In an embodiment, ligation of the further nucleic acid molecule to the nucleic acid substrate does not generate a circular ligation product. By circular ligation product, we include the meaning of one that has no exposed termini, and which has a continuous sugar-phosphate backbone.

In one embodiment, the further nucleic acid molecule is double stranded DNA, the nucleic acid substrate is a long terminal repeat (LTR) sequence that is selectively targeted by an integrase of the virus, and the nucleic acid-modifying enzyme is an integrase. In this case, the microorganism is selected from a virus, for example a retrovirus such as HIV, SIV, MLV or FIV. Preferably, the nucleic acid substrate is immobilised on a surface as described above. To enable detection of the ligation product, the ends of the double stranded DNA are designed in such a way that, following ligation, they can bind to oligonucleotides (e.g. to the so-called SHOfLE oligonucleotides as described below). In a more specific embodiment in the context when the nucleic acid substrate is a LTR and the nucleic acid-modifying enzyme is an integrase, the further nucleic acid molecule is double stranded DNA wherein the first strand comprises the sequence TGCACGCATGTCGATGTGTCGCAATCGCATGTTGTCATCGTGCATGCATCATGACG TGCTGACTGAAGCTTCCGCT (SEQ ID No: 12) and the second strand comprises the sequence TCAGCACGTCATGATGCATGCACGATGACAACATGCGATTGCGACACATCGACATG CGTGCAAGCGGAAGCTTCAG (SEQ ID No: 13); or wherein the first and second strands each comprise a sequence sharing at least 30% sequence identity thereto, such as at least 40%, 50%, 60%, 70%, 75%, 80%, 85%, 90%, 95% or 99% sequence identity thereto of any of said sequences. To the extent that the further nucleic acid molecule differs from the precise sequences above, it will be appreciated that it must still be ligated to the nucleic acid substrate in the presence of integrase to form a ligation product which comprises a linear single strand of nucleic acid that is capable of being detected. For detection by SHOfLE oligonucleotides described below, only the ends of the double stranded DNA is important, and so to the extent that the further nucleic acid molecule differs from the precise sequences above, it is preferred that up to the 15 (e.g. up to the 14, 13, 12, 11 or 10) nucleotides at the 5′ and 3′ terminal are unchanged (or else only contain up to 4 nucleotide substitutions).

In another embodiment, the further nucleic acid molecule is single stranded DNA, the nucleic acid substrate is single stranded DNA that is selectively targeted by a type I topoisomerase, and the nucleic acid-modifying enzyme is a type I topoisomerase. For example, the nucleic acid substrate may be one that is selectively targeted by Mycobacterium type I topoisomerase (e.g. Mycobacterium tuberculosis or Mycobacterium smegmatis), and the nucleic acid-modifying enzyme may be a Mycobacterium type I topoisomerase. In this case, the microorganism is a Mycobacterium, for example Mycobacterium tuberculosis or Mycobacterium smegmatis. In a more specific embodiment in the context when the nucleic acid substrate is single stranded DNA that is selectively targeted by a Mycobacterium type I topoisomerase and the nucleic acid-modifying enzyme is a type I topoisomerase, the further nucleic acid molecule comprises the sequence CAGTGAGCGTCTGGCTGAAGCTTCCGCT (SEQ ID No: 14), or a sequence sharing at least 30% sequence identity thereto, such as at least 40%, 50%, 60%, 70%, 75%, 80%, 85%, 90%, 95% or 99% sequence identity thereto; or a sequence which is a part of at least 10, 15, 20 or 25 consecutive nucleotides, of said sequence. To the extent that the further nucleic acid molecule differs from the precise sequence above, it will be appreciated that it must still be ligated to the nucleic acid substrate in the presence of a Mycobacterium type I topoisomerase to form a ligation product which comprises a linear single strand of nucleic acid that is capable of being detected.

In yet another embodiment, the further nucleic acid molecule is single stranded DNA, the nucleic acid substrate is double stranded DNA that is selectively targeted by a type I topoisomerase, and the nucleic acid-modifying enzyme is a type I topoisomerase. For example, the nucleic acid substrate may be one that is selectively targeted by Plasmodium type I topoisomerase (e.g. Plasmodium falciparum), and the nucleic acid-modifying enzyme may be a Plasmodium type I topoisomerase. In this case, the microorganism is a Plasmodium, for example Plasmodium falciparum. In a more specific embodiment in the context when the nucleic acid substrate is double stranded DNA that is selectively targeted by a Plasmodium type I topoisomerase and the nucleic acid-modifying enzyme is a type I topoisomerase, the further nucleic acid molecule comprises the sequence TTCTAGACCAGACGCTCACTGAGCGGAAGCTTCAG (SEQ ID No: 15), or a sequence sharing at least 30% sequence identity thereto, such as at least 40%, 50%, 60%, 70%, 75%, 80%, 85%, 90%, 95% or 99% sequence identity thereto; or a sequence which is a part of at least 10, 15, 20 or 25 consecutive nucleotides, of said sequence. To the extent that the further nucleic acid molecule differs from the precise sequence above, it will be appreciated that it must still be ligated to the nucleic acid substrate in the presence of a Plasmodium type I topoisomerase to form a ligation product which comprises a linear single strand of nucleic acid that is capable of being detected.

In yet a more specific embodiment in the context when the nucleic acid substrate is double stranded DNA that is selectively targeted by a Plasmodium type I topoisomerase and the nucleic acid-modifying enzyme is a type I topoisomerase, the further nucleic acid molecule may be, a part of the nucleic acid substrate. For example, the nucleic acid substrate may be double stranded DNA wherein the first strand comprises the sequence ACTACCATTCTGAGTCGTTCGATCTAAAAGACTTAGA (SEQ ID No: 3) and the second strand comprises the sequence ATTTTTCTAAGTCTTTTAGATCGAACGACTCAGAATG (SEQ ID No: 4), or sequences sharing at least 30% sequence identity thereto, such as at least 40%, 50%, 60%, 70%, 75%, 80%, 85%, 90%, 95% or 99% sequence identity thereto, or sequences which are a part of at least 5 consecutive nucleotides, such as at least 10, 15, 20, 25, 30 or 35 consecutive nucleotides, of any of said sequences; and the further nucleic acid molecule may comprise the first or second strand of that nucleic acid substrate. For instance, the further nucleic acid molecule may be the PCR product formed using the vector pYES2.1 TOPO (Invitrogen) as a template, and the primers P1: ATT TTT CTA AGT CTT TTA GAT CGA ACG ACT CAG AAT GAT GCA TGT ATA CTA AAC TCA CAA ATT AGA GC (SEQ ID No: 25) and P2: TTT TTT TTT TTT TTT TTT TTT TTT TGC TTT CTC ATA GCT CAC GCT G (SEQ ID No: 26), or a-sequence sharing at least 30% sequence identity thereto, such as at least 40%, 50%, 60%, 70%, 75%, 80%, 85%, 90%, 95% or 99% sequence identity thereto, or a sequence which is a part of at least 10, 15, 20, 25, 30, 35, 40, 45, 50, 100, 150 or 200 consecutive nucleotides, of said sequence. The generated PCR fragment is around 1500 bp and comprises the nucleotide sequence of SEQ ID No: 4 which hybridises to the nucleotide sequence of SEQ ID No: 3 to make up the nucleic acid substrate. To the extent that the nucleic acid substrate and further nucleic acid molecule differ from the precise sequences above, it will be appreciated that they must still be ligated to one another in the presence of a Plasmodium type I topoisomerase to form a ligation product which comprises a linear single strand of nucleic acid that is capable of being detected. It will be appreciated that in this embodiment, the further nucleic acid molecule may be ligated to the nucleic acid substrate to form a ligation product which comprises a linear single strand of nucleic acid that is capable of being detected, indirectly. Following ligation of one of the strands of the nucleic acid substrate to one of the strands of the PCR product, the strand of the PCR product that is ligated to the nucleic acid substrate is subsequently exposed when washed under denaturing conditions. This exposed strand then corresponds to the linear single strand of nucleic acid that is capable of being detected.

In still another embodiment, the further nucleic acid molecule is single stranded or double stranded DNA, the nucleic acid substrate is single stranded DNA that is selectively targeted by a type I topoisomerase, and the nucleic acid-modifying enzyme is a type I topoisomerase. For example, the nucleic acid substrate may be one that is selectively targeted by a Streptococcus type I topoisomerase (e.g. Group B Streptococcus (GBS), for example Streptococcus agalactiae), and the nucleic acid-modifying enzyme may be a Streptococcus type I topoisomerase (e.g. Group B Streptococcus (GBS), for example Streptococcus agalactiae). In this case, the microorganism is a Streptococcus, for example Group B Streptococcus (GBS), such as Streptococcus agalactiae. In a more specific embodiment in the context when the nucleic acid substrate is single stranded DNA that is selectively targeted by a Streptococcus type I topoisomerase and the nucleic acid-modifying enzyme is a type I topoisomerase, the further nucleic acid molecule may be the PCR product formed using the vector pYES2.1 TOPO (Invitrogen) as a template, and the primers P1: ATT TTT CTA AGT CTT TTA GAT CGA ACG ACT CAG AAT GAT GCA TGT ATA CTA AAC TCA CAA ATT AGA GC (SEQ ID No: 25) and P2: TTT TTT TTT TTT TTT TTT TTT TTT TGC TTT CTC ATA GCT CAC GCT G (SEQ ID No: 26), or a sequence sharing at least 30% sequence identity thereto, such as at least 40%, 50%, 60%, 70%, 75%, 80%, 85%, 90%, 95% or 99% sequence identity thereto, or a sequence which is a part of at least 10, 15, 20, 25, 30, 35, 40, 45, 50, 100, 150 or 200 consecutive nucleotides, of said sequence. To the extent that the further nucleic acid molecule differs from the precise sequence above, it will be appreciated that it must still be ligated to the nucleic acid substrate in the presence of a Streptococcus type l topoisomerase to form a ligation product which comprises a linear single strand of nucleic acid that is capable of being detected.

Preferably, the nucleic acid substrate is immobilised on a surface as described above, and once the further nucleic acid molecule has been allowed to ligate to it to form the ligation product, the surface is washed to remove substantially all further nucleic acid molecule that is non-ligated to the substrate. The surface can be washed by any liquid which does not disrupt the integrity of the ligation product immobilised on the surface. Suitable buffers are described in the Examples and include Tris based buffers and saline sodium citrate buffers.

Where the further nucleic acid molecule is part of the nucleic acid substrate, the nucleic acid substrate (including the further nucleic acid molecule) is preferably immobilised on a surface as described above (e.g. on a bead or slide such as a codelink slide).

By a ‘linear single strand of nucleic acid that is capable of being detected’, we include the meaning of the ligation product comprising an exposed single strand of nucleic acid that can bind to a detectable entity such as another oligonucleotide, a protein or a dye, as described further below. Such binding can be direct or indirect, for example via one or more linking moieties. The exposed single strand of nucleic acid can be a 3′ or 5′ overhang, for example. Generally, the exposed single strand of nucleic acid is between 5 and 50 nucleotides in length, such as between 20 and 40, or between 25 and 35 nucleotides in length.

The linear single strand of nucleic acid that is capable of being detected may also itself comprise one or more detectable oligonucleotides. This may be especially desirable in the embodiment where the further nucleic acid molecule is part of the nucleic acid substrate, and where the ligation product is formed indirectly via one or more processing steps, for example as illustrated in FIGS. 17 and 18(B). In this way, the presence of the detectable signal after the one or more processing steps (e.g. wash step) may provide a direct readout of the presence of the nucleic acid-modifying enzyme.

It is preferred if the linear single strand of nucleic acid in the ligation product is one that is capable of being detected by hybridising to one or more detectable oligonucleotides, and more preferably to one or more detectable oligonucleotides, herein defined as “SHOfLE oligonucleotides”.

By “SHOfLE oligonucleotides” we include the meaning of one or more detectable oligonucleotides that are capable of forming, by self-assembly with other such one or more detectable oligonucleotides, a pair of complementary, substantially contiguous single strands of a double-stranded nucleic acid filament. The single strands of the double-stranded nucleic acid filament may be termed plus and minus strands, or sense and antisense strands to reflect the complementary base pairing between nucleotides from the respective strands. By “substantially contiguous” we include the meaning of the confronting head-to-tail nucleotide bases in adjacent SHOfLE oligonucleotides from one strand, being immediately adjacent to one another, or being spaced (e.g. by up to 1-5 bases) such that each single strand is not necessarily continuous. In the latter case, each single strand will have periodic gaps that are offset from gaps in the other strand of the double stranded filament.

Generally, each SHOfLE oligonucleotide has two adjacent segments (i.e. a 5′ segment and a 3′ segment) that can hybridise to the same complementary segments in another SHOfLE oligonucleotide. Under hybridisation conditions, the first 5′-end segment in one SHOfLE oligonucleotide will hybridise to the first 5′-end segment in another SHOfLE oligonucleotide, and the second 3′-end segment in one SHOfLE oligonucleotide will hybridise to the second 3′-end segment in another SHOfLE oligonucleotide. In this way, the SHOfLE oligonucleotides self-assemble to form a double stranded polynucleotide in which the first and second segments of an oligonucleotide in one strand of the filament are hybridised with complementary first and second segments, respectively, of two adjacent oligonucleotides in the second strand of the filament. In other words, the SHOfLE oligonucleotides in one strand of the double stranded filament are hybridised to other SHOfLE oligonucleotides in the other strand of the filament in a staggered fashion wherein one oligonucleotide on one strand of the filament is hybridised to two oligonucleotides on the other side of the filament. An illustration of the self-assembly of such SHOfLE oligonucleotides is provided in FIG. 14.

It will be appreciated that the first and second segments of the SHOfLE oligonucleotides may be the same or different lengths. Equally, the SHOfLE oligonucleotides used to detect the linear single strand of nucleic acid in the ligation may comprise a single oligonucleotide species, wherein each SHOfLE oligonucleotide has the same sequence and length, or it may comprise multiple (e.g. 2, 3, 4 or more) oligonucleotide species of different sequences and lengths. In any event, the SHOfLE oligonucleotides are ones that self-assemble under hybridisation conditions to form the double stranded nucleic acid filaments described above, that are capable of being detected.

Typical lengths of SHOfLE oligonucleotide range from 6 to 50 nucleotides in length, such as between 10-20, 10-30, 10-40, 10-50, 20-40 and 20-30 nucleotides in length. In one embodiment, the SHOfLE oligonucleotides are between 25 and 30 nucleotides in length, such as 26, 27, 28 or 29 nucleotides in length.

Examples of SHOfLE oligonucleotides include CAG TGA GCG TCT GGC TGA AGC TTC CGC T (SEQ ID No: 16) and CCA GAC GCT CAC TGA GCG GAA GCT TCA G (SEQ ID No: 17). Other such SHOfLE oligonucleotides that have the functions described above can be readily designed in line with standard molecular biology techniques and principles. Preferably, the SHOfLE oligonucleotides are ones that have minimal second structure so as to facilitate creation of the double stranded nucleic acid filament.

Where the the linear single strand of nucleic acid in the ligation product is detected by binding to a detectable oligonucleotide (e.g. a SHOfLE oligonucleotide), the oligonucleotide may hybridise to that strand of nucleic acid directly, in which case the sequence of the detectable oligonucleotide (e.g. SHOfLE oligonucleotide) should of course may complementary to at least part of the sequence of the linear single strand of nucleic acid. The design of such oligonucleotides is well known to those of skill in the art. Detectable oligonucleotides of suitable lengths are within the scope of the invention, and the lengths are typically similar to the single, of the linear single strand of nucleic acid, although it is appreciated that they may be longer or shorter than the linear single strand of nucleic acid.

Binding of the one or more detectable nucleotides to the linear single strand of nucleic acid may be detected directly. For example, the detectable nucleotide may be a labelled nucleic acid probe. Alternatively, binding of the one or more detectable nucleotides to the linear single strand of nucleic acid may allow for the generation of an amplification product (e.g. a PCR product or assembly of SHOfLE oligonucleotides as defined herein). Essentially any suitable detection method can be used to detect the linear single strand of nucleic acid, including one or more techniques selected from the group consisting of a linear amplification technique, southern blotting, polymerase chain reaction (PCR), reverse-transcription-PCR (RT-PCT), quantitative PCR (qPCR), restriction fragment length dimorphism-PCR (RFLD-PCR), primer extension, DNA array technology, and isothermal amplification, all of which are standard practice in the art.

Conveniently, the one or more detectable oligonucleotides (e.g. SHOfLE oligonucleotides) are detectably labelled with a detectable moiety such as one or more fluorescent dyes, one or more other dyes, one or more moieties that allow detection by silver staining, radioactive nucleotides and/or biotinylated nucleotides. The detectable moiety may be bound directly to, or else incorporated in, the oligonucleotide (e.g. in the case of a fluorescent dye or radioactive nucleotides) or it may be bound indirectly to the oligonucleotide (e.g. biotinylated nucleotides may be detected by using streptavidin gold nanoparticle-enhanced silver staining). In this way, the presence of the ligation product can be qualified and quantified by assessing a readily detectable signal such as any of a radioactive signal or a fluorescent signal or a chemiluminescent signal or a colometric signal.

It will also be appreciated that the one or more detectable oligonucleotides may be coupled to an enzyme, which enzyme is capable of converting a substrate into a detectable product. The enzyme may, for example, be fused with streptavidin, thereby enabling it to be coupled to biotinylated nucleotides. The enzyme may be any enzyme with an easily detectable activity. In one example, the enzyme is horse-radish peroxidase, a substrate of which is TMB (3,3′,5,5′-Tetramethylbenzidine).

In a preferred embodiment, the one or more detectable oligonucleotides are SHOfLE oligonucleotides that are detectably labelled with a detectable moiety (e.g. one or more fluorescent dyes) or that contain biotinylated nucleotides that can be detected using streptavidin gold nanoparticle-enhanced silver staining. In this way, there is no requirement for a polymerase enzyme. This improves the stability of the reaction mixture, and also results in a cheaper assay. Hence, in one embodiment, the presence of the ligation product is detected by an amplification technique that does not involve use of a polymerase.

In one embodiment, the presence of the ligation product is detected in a microfluidic system. Thus, the method may comprise:

(i) loading the sample, the nucleic acid substrate, and the further nucleic acid into a sample chamber comprising a flow through channel, wherein droplets comprising the sample, nucleic acid substrate and further nucleic acid molecule are generated;

(ii) transferring the droplets from the sample chamber to a droplet retaining means through the flow through channel;

(iii) capturing one or more single droplets in individual cavities of the droplet retaining means, wherein each single droplet is spatially isolated from other droplets; and

(iv) detecting, in one or more captured droplets, the ligation product.

It will be appreciated that when the nucleic acid substrate is immobilised on a surface, which surface is washed following contact with the sample, the method may comprise:

(i) loading the washed nucleic acid substrate and the further nucleic acid molecule into a sample chamber comprising a flow through channel, wherein droplets comprising the nucleic acid substrate are generated;

(ii) transferring the droplets from the sample chamber to a droplet retaining means through the flow through channel;

(iii) capturing one or more single droplets in individual cavities of the droplet retaining means, wherein each single droplet is spatially isolated from other droplets; and

(iv) detecting, in one or more captured droplets, the ligation product.

In an embodiment of the invention where a microfluidic system is used, the sample chamber comprises one or more inlet channels and/or one of more outlet channels for the generated drops. The one or more flow through channels, inlet channels and/or outlet channels have a diameter of 10-50 micrometers, such as approximately 25 micrometers.

Further detail of microfluidics systems is provided in document WO 2013/029631, which is incorporated herein by reference.

The invention provides a method of identifying a microorganism that expresses a nucleic acid-modifying enzyme, in a sample, the method comprising:
(a) contacting a nucleic acid substrate targeted by the nucleic acid-modifying enzyme with the sample, wherein the nucleic acid substrate is capable of being processed in the presence of the nucleic acid modifying enzyme to form a ligation product which comprises a linear single strand of nucleic acid that is capable of being detected, and wherein the nucleic acid substrate is immobilised on a surface such as a bead or slide (e.g. a codelink slide);
(b) washing the nucleic acid substrate immobilised on the surface so as to remove substantially all non-specifically bound material from the substrate; and
(c) detecting the presence of the ligation product.

Preferably, the nucleic acid substrate is double stranded nucleic acid whereby, in the presence of the nucleic acid-modifying enzyme, a first strand is capable of being ligated to a second strand to form a ligation product which comprises a linear single strand of nucleic acid that is capable of being detected. The first strand may be immobilised to the surface such as a bead or slide (e.g. a codelink slide) and the second strand may be hybridised to the first strand without being immobilised to the surface. Thus, the invention provides a method of identifying a microorganism that expresses a nucleic acid-modifying enzyme, in a sample, the method comprising:
(a) contacting a nucleic acid substrate targeted by the nucleic acid-modifying enzyme with the sample, wherein the nucleic acid substrate comprises, a first strand of nucleic acid that is immobilised on a surface (e.g. bead or slide (e.g. a codelink slide) and a second strand of nucleic acid that is hybridised to the first strand of nucleic acid but which is not immobilised to the surface, and wherein, in the presence of the nucleic acid modifying enzyme, the first strand of nucleic acid is capable of being ligated to the second strand of nucleic acid to form a ligation product which comprises a linear single strand of nucleic acid that is capable of being detected,
(b) washing the nucleic acid substrate immobilised on the surface so as to remove substantially all non-specifically bound material from the substrate, under denaturing conditions; and
(c) detecting the presence of the ligation product.

It will be appreciated that the wash step (b) may involve exposing an intermediate product formed by ligation of a first and second strand of the nucleic acid substrate, to denaturing conditions (e.g. urea such as 6-8M urea) such that a linear single strand of nucleic acid is exposed. This is illustrated in FIGS. 17 and 18(B).

In one embodiment, the bead is a streptavidin coated bead and the first strand of the nucleic acid substrate is biotinylated.

In one embodiment, the slide is a codelink slide and the first strand of the nucleic acid substrate is amine linked.

It will be appreciated that the method of the first aspect of the invention may be used for identifying any microorganism and/or any infection in any subject.

Accordingly, a second aspect of the invention provides a method of diagnosing an infectious disease in a subject, the method comprising identifying a microorganism in a sample from the subject according to the method of the first aspect of the invention, wherein the presence of the microorganism in the sample is indicative of the infectious disease.

The infectious disease may be any disease in which at least part of the pathology is mediated by the presence of a microorganism (e.g. pathogen or parasite), such as any of those described above. For example, the infectious disease may be a bacterial infection, a viral infection, a protozoan infection or a fungal infection. A large number of microorganisms are known to be associated as causative agents with certain infectious diseases. Preferably, the infectious disease is tuberculosis, in which case the method of the first aspect of the invention is used to detect Mycobacterium tuberculosis (e.g. by identifying the activity of a Mycobacterium type I topoisomerase). Also preferably, the infectious disease is malaria, in which case the method of the first aspect of the invention is used to detect Plasmodium falciparum (e.g. by identifying the activity of a Plasmodium falciparum type I topoisomerase). Yet further preferably, the infectious disease is one caused by a virus, such as a retrovirus. An example of a preferred disease is HIV infection, SIV infection, MLV infection, and FIV infection. The infection may result in AIDs. Thus, the infectious disease may be a retroviral infection (e.g. a HlV, SIV, MLV or FIV infection), in which case the method of the first aspect of the invention is used to detect a retrovirus (e.g. HIV, SIV, MLV or FIV), for example by identifying the activity of a retroviral integrase (e.g. HIV, SIV, MLV or FIV integrase). Viruses, such as retroviruses, have been linked to a wide range of cancers, and so the when the microorganism is a virus (e.g. a retrovirus), the disorder may be a cancer. For example leukaemia (e.g. chronic and acute) is known to be associated with retroviral infection. Still further, preferably, the infectious disease is Group B Streptococcal disease (GBS disease e.g. perinatal Group B Streptococcal disease)

Preferences for the subject include those described above, for example the subject can be any of a mammal, a human, a cow, a ferret, a badger, a koala, a chicken, a turkey, a duck, a rodent, an elephant, a bird, a pig, a deer, a coyote, a camel, a puma, a fish, a dog, a sheep, a goat, a cat, or a non-human primate. Preferably, the subject is, a human. It is known that some infectious diseases affect some subjects more than others, and, so the subject is typically one that is known to be commonly affected by the infectious disease in question. For example in the case of tuberculosis, the subject is commonly a human or a cow. For example, in the case of GBS, the subject is commonly a pregnant female (e.g. a human).

Also, the microorganisms identified are explained elsewhere herein, for example the microorganism may be selected from the Plasmodium and/or Mycobacterium and/or Streptococcus genus. In a preferred embodiment, the infectious disease is malaria, and the microorganism is selected from the Plasmodium genus, for example, the microorganism is Plasmodium falciparum. In another preferred embodiment, the infectious disease;is human and/or bovine tuberculosis, and the microorganism is selected from the Mycobacterium genus, for example the microorganism is Mycobacterium tuberculosis. In another preferred embodiment, the infectious disease is Group B Streptococcal disease (e.g. perinatal GBS disease), and the microorganism is a Streptococcus, for example Group B Streptococcus such as Streptococcus agalactiae.

The diagnostic applications of the present invention may be practised using any suitable and practical setup or machinery, which utilises the system's sensitivity, simplicity and short reaction time. For example, the method may be used in advanced equipment for single cell or single molecule detection for ultra-sensitive detection of infection sources. Alternatively, the diagnostic methods may be performed in the style of stick tests/dipsticks. A testing dipstick can be made of paper or cardboard and is impregnated with the reagents required to perform the reaction of the invention. The readout of a dipstick test is preferably presented by a changing colour. In this way, dipsticks can be used to test for a variety of liquid samples for the presence of a specific microorganism, and the dipstick can then be employed in easy and efficient diagnosis of infectious diseases, such as any infectious disease according to the present invention.

A third aspect of the invention provides a method of combating an infectious disease in a subject, the method comprising diagnosing the disease according to the method of the second aspect of the invention, and treating the disease.

The invention includes an anti-infectious disease agent for use in combating an infectious disease in a subject who has been diagnosed with the disease according to the method of the second aspect of the invention.

Similarly, the invention includes the use of an anti-infectious disease agent in the manufacture of a medicament for combating an infectious disease in a subject who has been diagnosed with the disease according to the method of the second aspect of the invention.

By combating an infectious disease, we include the meaning of reducing or alleviating symptoms in a subject (i.e. palliative use), preventing symptoms from worsening or progressing, and treating the disorder (e.g. by inhibition or elimination of the causative agent).

Preferences for the infectious disease include those mentioned above in relation to the second aspect of the invention.

The treatment of the disease encompasses any known treatment regime, or administration of one or more suitable therapeutic agents, for the infectious disease in question. Thus, when the infectious disease is a bacterial infection, the method may include administering one or more antibiotics; when the infectious disease is a virus, the method may include administering one or more anti-viral agents; when the infectious disease is a fungal infection, the method may include administering one or more anti-fungal agents; and when the infectious disease is a protozoan infection, the method may include administering one more anti-protozoan agents; and so on. When the disease is a cancer, the method may include administering one or more anti-cancer agents.

As a particular example, when the disease is tuberculosis, the method may comprise administering one or more anti-tuberculosis agents selected from the group consisting of ethambutol, isoniazid, pyrazinamide, rifampicin, streptomycin, aminoglycoside, amikacin, kanamycin, capreomycin, viomycin, enviomycin, fluoroquinolone, ciprofloxacin (CIP), levofloxacin, moxifloxacin, thioamide, ethionamide, prothionamide, cycloserine, terizidone, rifabutin, macrolide, clarithromycin, linezolid, thioacetazone, thioridazine, arginine, vitamin D, and bedaquiline.

As another example, when the disease is malaria, the method may comprise administering one or more anti-malarial agents selected from the group consisting of an artemisinin, amodiaquine, lumefantrine, mefloquine, sulfadoxine/pyrimethamine, dihydroartemisinin, piperaquine, quinine, and clindamycin.

As yet another example, when the disease is HIV, the method may comprise administering one or more anti-HIV agents selected from the group consisting of a nucleoside reverse transcriptase inhibitor (e.g. abacavir, didanosine, emtricitabine, lamivudine, stavudine, tenofovir disoproxil fumarate, zidovudine), a non-nucleoside reverse transcriptase inhibitor (e.g. delavirdine, efavirenz, etravirine, nevirapine, rilpivirine), a proteasome inhibitor (e.g. atazanavir, darunavir, fosamprenavir, indinavir, nelfinavir, ritonavir, saquinavir, tipranavir), a fusion inhibitor (e.g. enfuvirtide), an entry inhibitor (e.g. maraviroc), and an integrase inhibitor (e.g. dolutegravir and raltegravir).

As yet another example, when the disease is Group B Streptococcal disease, the method may comprise administering one or more antibiotics known to be effective in treating GBS disease, for example benzylpenicillin or ampicillin plus gentamicin.

As another example, when the disease is cancer, the method may comprise administering one or more anti-cancer agents (e.g. chemotherapeutic agents). For instance, when the cancer is acute leukaemia, the anti-cancer agent may be all trans retinoic acid (ATRA).

The nucleic acid-modifying enzymes described herein are often essential to the life-cycle of microorganisms since they are part of DNA metabolism. It follows that any agent capable of specifically blocking, inhibiting or down-regulating the activity of these nucleic acid-modifying enzymes may be used as therapeutic agents against such microorganisms and infectious diseases caused by such microorganisms.

Accordingly, a fourth aspect of the invention provides a method of assessing whether an agent has an effect on an infectious disease caused by a microorganism in a subject, the method comprising: (a) administering the agent to the subject and (b) determining the effect of the agent on the amount of ligation product produced in a sample from the subject when the sample is contacted with a nucleic acid substrate targeted by a nucleic acid-modifying enzyme of the microorganism and a further nucleic acid molecule that is ligated to the nucleic acid substrate in the presence of the nucleic acid-modifying enzyme, according to the method of the first aspect of the invention. It will be appreciated that step (b) may be performed separately from step (a) if step (a) has already been carried out and a sample, taken from the subject following administration of the agent, is available.

Similarly, the invention provides a method of assessing whether an agent has an effect on a microorganism in a sample, the method comprising:

(a) contacting the sample with the agent; and

(b) assessing the effect of the agent on the amount of ligation product produced when the sample is contacted with a nucleic acid substrate targeted by a nucleic acid-modifying enzyme of the microorganism and a further nucleic acid molecule that is ligated to the nucleic acid substrate in the presence of the nucleic acid-modifying enzyme, according to the method of the first aspect of the invention. Thus, the invention provides a method of assessing whether an agent has an effect on a microorganism in a sample, the method comprising:

(a) providing a sample;

(b) providing a nucleic acid substrate targeted by a nucleic acid-modifying enzyme of the microorganism;

(c) providing a further nucleic acid molecule that is ligated to the nucleic acid substrate in the presence of the nucleic acid-modifying enzyme;

(d) providing an agent

(d) combining the sample of step (a), the nucleic acid substrate of step (b) and the further nucleic acid molecule of step (c), which combination is done with or without the agent of step (d); and

(e) detecting the ligation product produced in step (d), with or without the agent.

It will be understood that the further nucleic acid molecule may be part of the nucleic acid substrate as described above.

It is appreciated that the agent may either enhance, reduce or eliminate production of the ligation product depending on its effect on the microorganism. For example, if the agent is detrimental to the growth and/or survival of the microorganism, the agent would be expected to reduce or eliminate production of the ligation product. Similarly, if the agent activates the growth and/or survival of the microorganism, the agent would be expected to enhance production of the ligation product. Thus, the methods of the invention encompass both positive and negative effects.

Typically, however, the methods of the fourth aspect of the invention are employed to identify agents that have a negative effect on an infectious disease in a subject or on a microorganism. In other words, the methods of the fourth aspect of the aspect may be used to identify potential therapeutic agents that inhibit the growth and/or survival of the microorganism.

Preferences for the sample, microorganism, nucleic acid substrate, nucleic acid-modifying enzyme, and further nucleic acid for this and all subsequent aspects of the invention include those described above in relation to the first aspect of the invention.

In a particular example, the methods may be used to assess whether an agent has an effect (e.g. an inhibitory effect) on an infectious disease caused by a Plasmodium (e.g. malaria), or on a Plasmodium microorganism (e.g. Plasmodium falciparum), by assessing the effect of the agent on the amount of ligation product produced when a nucleic acid substrate that is selectively targeted by a Plasmodium type I topoisomerase is brought into contact with the sample, and with a further nucleic acid molecule that is ligated to the nucleic acid substrate in the presence of a Plasmodium type I topoisomerase.

In another example, the methods may be used to assess whether an agent has an effect (e.g. an inhibitory effect) on an infectious disease caused by a Mycobacterium (e.g. tuberculosis), or on a Mycobacterium microorganism (e.g. Mycobacterium tuberculsosis), by assessing the effect of the agent on the amount of ligation product produced when a nucleic acid substrate that is selectively targeted by a Mycobacterium type I topoisomerase is brought into contact with the sample, and with a further nucleic acid molecule that is ligated to the nucleic acid substrate in the presence of a Mycobacterium type I topoisomerase.

In yet another example, the methods may be used to assess whether an agent has an effect (e.g. an inhibitory effect) on an infectious disease caused by a retrovirus (e.g. HIV, SIV, MLV or FIV), or on a retrovirus microorganism (e.g. HIV, SIV, MLV or Fly), by assessing the effect of the agent on the amount of ligation product produced when a nucleic acid substrate that is selectively targeted by a retroviral integrase is brought into contact with the sample, and with a further nucleic acid molecule that is ligated to the nucleic acid substrate in the presence of a retroviral integrase.

In still yet another example, the methods may be used to assess whether an agent has an effect (e.g. an inhibitory effect) on an infectious disease caused by a Streptococcus (e.g. Group B Streptococcus such as Streptococcus agalactiae), by assessing the effect of the agent on the amount of ligation product produced when a nucleic acid substrate that is selectively targeted by a Streptococcus type I topoisomerase (e.g. Group B Streptococcus type I topoisomerase) is brought into contact with the sample, and with a further nucleic acid molecule that is ligated to the nucleic acid substrate in the presence of a Streptococcus type I topoisomerase (e.g. Group B Streptococcus type I topoisomerase).

Several techniques are available in the art to detect the presence of the ligation product and are discussed above in relation to the first aspect of the invention. Many of the techniques allow quantification of the amount of ligation product produced. For example binding of detectable moieties to the linear single strand of nucleic acid of the ligation product generally results in a detectable signal that can be readily quantified (e.g. a radioactive signal or a fluorescent signal or a chemiluminescent signal or a colometric signal). Alternatively, in some embodiments, the linear single strand of nucleic acid may itself comprise one or more detectable nucleotides.

It will be appreciated that it may be desirable to assess the amount of ligation product formed in the absence of the agent, to provide a baseline level to which the amount of ligation product formed in the presence of the agent can be compared to. For example, where the method comprises administering the agent to the subject and assessing whether that agent has any effect on the amount of ligation product produced in a sample taken from the subject when that sample is contacted with the nucleic acid substrate and further nucleic acid molecule, it may be desirable to take two samples from the subject for the assay. A first sample may be taken before the subject is administered the agent, and a second sample may be taken after the subject has been administered the agent. The nucleic acid substrate and further nucleic acid molecule can then be added to both samples and the samples incubated under conditions that support ligation, and the amount of ligation product formed assessed. If the amount of ligation product formed is less in the second sample, then the agent is expected to have a negative effect (e.g. an inhibitory or suppressing effect) on the particular microorganism, whereas if the amount of ligation product is more in the second sample, then the agent is expected to have a positive effect (e.g. a stimulatory effect) on the particular microorganism. Similarly, where the method comprises contacting the sample with the agent directly, it may be desirable to assess the amount of ligation product formed when the nucleic acid substrate and further nucleic acid molecule is added to the sample before the agent is added, and repeat the assessment after the agent is added.

However, it will also be appreciated that the baseline amount of ligation product may already be known, and so there is no need to perform another assay to calculate it.

The above aspect of the invention includes screening methods to identify drugs or lead compounds of use in treating a disease or condition caused by the microorganism (e.g. malaria or tuberculosis or GBS disease). It is appreciated that screening assays which are capable of high throughput operation are particularly preferred.

It is appreciated that in the methods described herein, which may be drug screening methods, a term well known to those skilled in the art, the agent may be a drug-like compound or lead compound for the development of a drug-like compound.

The term “drug-like compound” is well known to those skilled in the art, and may include the meaning of a compound that has characteristics that may make it suitable for use in medicine, for example as the active ingredient in a medicament. Thus, for example, a drug-like compound may be a molecule that may be synthesised by the techniques of organic chemistry, less preferably by techniques of molecular biology or biochemistry, and is preferably a small molecule, which may be of less than 5000 daltons and which may be water-soluble. A drug-like compound may additionally exhibit features of selective interaction with a particular protein or proteins and be bioavailable and/or able to penetrate target cellular membranes or the blood:brain barrier, but it will be appreciated that these features are not essential.

The term “lead compound” is similarly well known to those skilled in the art, and may include the meaning that the compound, whilst not itself suitable for use as a drug (for example because it is only weakly potent against its intended target, non-selective in its action, unstable, poorly soluble, difficult to synthesise or has poor bioavailability) may provide a starting-point for the design of other compounds that may have more desirable characteristics.

Thus in one embodiment the method further comprises modifying an agent which has been shown to modulate (e.g. decrease) the amount of ligation product produced, and testing the ability of the modified agent to modulate (e.g. decrease) the amount of ligation product produced.

The method may further comprise testing the ability of the modified agent to modulate at least one activity or function of the nucleic acid-modifying enzyme of the microorganism (e.g. a Plasmodium type I topoisomerase. a Mycobacterium type I topoisomerase, a Streptococcus type I topoisomerase and a retroviral integrase).

Once an agent or modified agent which modulates (e.g. decreases) the amount of ligation product produced has been found, it may be desirable to test its effect in a suitable model in vivo. For example, the method may comprise testing the ability of the agent or modified agent to modulate the amount of ligation product formed in a sample taken from an in vivo model of an infectious disease caused by the relevant microorganism. For example, the effect of the agent or modified agent may be tested in an animal model of malaria or tuberculosis or GBS disease or a retroviral infection (e.g. HIV, MLV, SIV, or FIV) or cancer.

The method may further comprise determining whether the agent or modified agent modulates or affects disease severity, duration or progression in the in vivo model of the infectious disease, such as malaria, tuberculosis or GBS disease or a retroviral infection (e.g. HIV, MLV, SIV or FIV) or cancer.

In a further embodiment, the method may also comprise the step of formulating an agent which has the ability to modulate (e.g. decrease) the amount of ligation product formed, into a pharmaceutically acceptable composition.

Therefore, the invention also includes a pharmaceutical composition comprising an agent that has an effect on an infectious disease in a subject or a microorganism in a sample (e.g. a suppressing or inhibitory effect) that has been identified as described above, and a pharmaceutically acceptable carrier, diluent or excipient.

While it is possible for an agent to be administered alone, it is preferable to present it as a pharmaceutical formulation, together with one or more acceptable carriers. The carrier(s) must be “acceptable” in the sense of being compatible with the agent of the invention and not deleterious to the recipients thereof. Typically, the carriers will be water or'saline which will be sterile and pyrogen free.

The aforementioned agents or a formulation thereof may be administered by any conventional method including oral, which is preferred, as well as parenteral (e.g. subcutaneous or intramuscular) injection. A suitable method of administration is intranasal or inhalation administration. Here, the agent or formulation is conveniently delivered in the form of a dry powder inhaler or an aerosol spray presentation from a pressurised container, pump, spray or nebuliser with the use of a suitable propellant, e.g. dichlorodifluoromethane, trichlorofluoromethane, dichlorotetrafluoro-ethane, a hydrofluoroalkane such as 1,1,1,2-tetrafluoroethane (HFA 134A3 or 1,1,1,2,3,3,3-heptafluoropropane (HFA 227EA3), carbon dioxide or other suitable gas. The pressurised container, pump, spray or nebuliser may contain a solution or suspension of the active compound, e.g. using a mixture of ethanol and the propellant as the solvent, which may additionally contain a lubricant, e.g. sorbitan trioleate. Capsules and cartridges (made, for example, from gelatin) for use in an inhaler or insufflator may be formulated to contain a powder mix of a formulation in accordance with the present invention and a suitable powder base such as lactose or starch. The aforementioned agents or formulation thereof can also be delivered using ultrasonic nebulisation techniques.

The treatment may consist of a single dose or a plurality of doses over a period of time.

It will be appreciated that the methods described herein need not be limited to the context of microorganisms and infectious diseases, but can be used to identify any nucleic acid-modifying enzyme in a sample. By measuring the activity of a nucleic acid-modifying enzyme on a given nucleic acid substrate, the present methods allows the detection of a nucleic acid-modifying enzyme in any sample, and by measuring the level of the activity one can get an insight into the amount of nucleic acid-modifying enzyme there is in a given sample. Thus, the methods of the present invention allow both a qualitative and quantitative assessment of a nucleic acid-modifying enzyme in a given sample.

Accordingly, a fifth aspect of the invention provides a method of assessing the presence or activity of a nucleic acid-modifying enzyme in a sample, the method comprising:

(a) contacting a nucleic acid substrate targeted by the nucleic acid-modifying enzyme with the sample, wherein the nucleic acid substrate is immobilised on a surface;

(b) adding a further nucleic acid molecule to the sample which nucleic acid molecule is ligated to the nucleic acid substrate in the presence of the nucleic acid-modifying enzyme to form a ligation product that comprises a linear single strand of nucleic acid that is capable of being detected; and

(c) detecting the presence or activity of the ligation product.

Preferences for the sample, nucleic acid substrate, nucleic acid-modifying enzyme, further nucleic acid, and detection methods, include those described above in relation to the first aspect of the invention. Preferably the ligation product is detected using the SHOfLE oligonucleotides defined herein.

It is preferred if the nucleic acid substrate is immobilised on a surface, and prior to step (b), the nucleic acid substrate on the surface is washed so as to remove substantially all non-specifically bound material from the substrate. Thus, it will be appreciated that the invention includes a method of assessing the presence or activity of a nucleic acid-modifying enzyme in a sample, the method comprising:

(a)(i) contacting a nucleic acid substrate targeted by the nucleic acid-modifying enzyme with the sample, wherein the nucleic acid substrate is immobilised on a surface;

(a)(ii) washing the nucleic acid substrate immobilised on the surface so as to remove substantially all non-specifically bound material from the substrate;

(b) adding a further nucleic acid molecule to the sample which nucleic acid molecule is ligated to the nucleic acid substrate in the presence of the nucleic acid-modifying enzyme to form a ligation product that comprises a linear single strand of nucleic acid that is capable of being detected; and

(c) detecting the presence or activity of the ligation product.

Suitable means for immobilising the nucleic acid substrate are described above in relation to the first aspect of the invention.

As described above in relation to the first aspect of the invention, it will be appreciated that the further nucleic acid molecule may be a part of the nucleic acid substrate.

Thus, the invention also provides a method of assessing the presence or activity of a nucleic acid-modifying enzyme in a sample, the method comprising:
(a) contacting a nucleic acid substrate targeted by the nucleic acid-modifying enzyme with the sample, wherein the nucleic acid substrate is capable of being processed in the presence of the nucleic acid modifying enzyme to form a ligation product which comprises a linear single strand of nucleic acid that is capable of being detected; and
(b) detecting the presence or activity of the ligation product.

Conveniently, the nucleic acid substrate is immobilised on to a surface (e.g. a bead or slide such as a codelink slide) Thus, the invention also provides a method of assessing the presence or activity of a nucleic acid-modifying enzyme in a sample, the method comprising:
(a) contacting a nucleic acid substrate targeted by the nucleic acid-modifying enzyme with the sample, wherein the nucleic acid substrate is capable of being processed in the presence of the nucleic acid modifying enzyme to form a ligation product which comprises a linear single strand of nucleic acid that is capable of being detected, and wherein the nucleic acid substrate is immobilised on a surface;
(b) washing the nucleic acid substrate immobilised on the surface so as to remove substantially all non-specifically bound material from the substrate; and
(c) detecting the presence or activity of the ligation product.

Preferably, the nucleic acid substrate is double stranded nucleic acid whereby, in the presence of the nucleic acid-modifying enzyme, a first strand is capable of being ligated to a second strand to form a ligation product which comprises a linear single strand of nucleic acid that is capable of being detected. The first strand may be immobilised to a surface such as a bead or slide (e.g. a codelink slide) and the second strand may be hybridised to the first strand without being immobilised to the surface. Thus, the invention provides a method of assessing the presence or activity of a nucleic acid-modifying enzyme in a sample, the method comprising:
(a) contacting a nucleic acid substrate targeted by the nucleic acid-modifying enzyme with the sample, wherein the nucleic acid substrate comprises a first strand of nucleic acid that is immobilised on a surface such as a bead or slide (e.g. a codelink slide) and second strand of nucleic acid that is hybridised to the first strand of nucleic acid but which is not immobilised to the surface, and wherein, in the presence of the nucleic acid modifying enzyme, the first strand of nucleic acid is capable of being ligated to the second strand of nucleic acid to form a ligation product which comprises a linear single strand of nucleic acid that is capable of being detected,
(b) washing the nucleic acid substrate immobilised on the surface so as to remove substantially all non-specifically bound material from the substrate, under denaturing conditions; and
(c) detecting the presence or activity of the ligation product.

The method may make use of a microfluidic system as outlined above and as described in WO2013/029631.

It will be appreciated that this aspect of the invention may be used to identify specific cells or cell types which express the nucleic acid-modifying enzymes in question, or which express said enzymes at a particular level. In other words, the method allows one to qualitatively and/or quantitatively assess a nucleic acid-modifying enzyme (e.g. topoisomerase, such as from Mycobacterium tuberculosis or Plasmodium falciparum or Streptococcus (e.g. Group B Streptococcus such as Streptococcus agalactiae), or a retroviral integrase such as from HIV, SIV, FIV or MLV, in a given sample, whereby either its presence in that sample can be detected, or whereby its presence and the activity associated with that presence can be detected.

The cell may be any suitable cell or cell type that expresses the nucleic acid-modifying enzyme including any of those mentioned above. For example, the cell or cell type may also be a cancer cell, which expresses a specific enzymatic activity such as a specific topoisomerase I activity. In this case, the method of the invention may be used for diagnosing a cancer, or staging a cancer on the basis of the expression of specific nucleic acid-modifying enzymes (e.g. if the cancer cells express unique nucleic acid-modifying enzymes, different from those expressed by non-cancer cells), or by relative activity of nucleic acid-modifying enzymes (e.g. if cancer and non-cancer cells express the same nucleic acid-modifying enzymes but which have different activities). The method can be employed for analysing the relative or absolute level of cancer cells in a tumour, which express a certain enzyme or has a certain enzymatic activity.

When the method is used to identify specific cells or cell types, the sample is preferably one that contains cells. The cells may be specific cells of the subject. For example, in one embodiment, the cells are cancer cells such as cancer cells isolated from a human being.

When the method is used to identify specific cells or cell types that express particular nucleic acid-modifying enzymes (e.g. type I topoisomerases or retroviral integrases), it is preferred if the nucleic acid substrate that is used is one which is selectively targeted by the nucleic acid-modifying enzyme from those cells or cell types. In other words, the nucleic acid substrate is targeted by nucleic acid-modifying enzymes (e.g. type I topoisomerases or retroviral integrases) from those specific cells or cell types, more than it is targeted by any other nucleic acid-modifying enzymes, for example nucleic acid-modifying enzymes of other cells or cell types. Preferably, the nucleic acid substrate is predominantly targeted by a nucleic acid-modifying enzyme (e.g. type I topoisomerase or retroviral integrase) of the specific cell or cell type, and to a lesser extent by any other nucleic acid-modifying enzyme that may be present in the sample, for example which originates from another cell or cell type in the sample. Thus, when the cell to be identified is a cancer cell, the nucleic acid substrate may be one that is selectively targeted by a nucleic acid-modifying enzyme (e.g. topoisomerase) expressed in a cancer cell, as opposed to a nucleic acid-modifying enzyme expressed in a non-cancer cell. However, it is understood that the cancer cell to be identified may not contain a unique nucleic acid-modifying enzyme (e.g. topoisomerase) that is not present in non-cancer cells, in which case the relative activities of the nucleic acid-modifying enzyme (e.g. topoisomerase) as between cancer cells and non-cancer cells may be used to identify the cancer cells. For example, if the cancer cells have a relatively high enzymatic activity, the activity of the nucleic acid-modifying enzyme (e.g. topoisomerase) in the sample can be used to assess the presence of the cancer cells, for example by comparing the activity with the activity expected for the same nucleic acid-modifying enzyme (e.g. topoisomerase) in non-cancer cells.

It will be appreciated that the method of the invention may also be used for sorting cells on the basis of their enzymatic expression profile, for example for sorting cells on the basis of a cancer tumour into a separate population on the basis of their enzymatic activities, for example the activity and specificity of type I topoisomerases of different cells of the tumour.

It will be appreciated that the method allows one to assess whether an agent has an effect on the activity of a nucleic acid-modifying enzyme in a sample, for example in a method comprising:

(a) contacting the sample with the agent, a nucleic acid substrate targeted by the nucleic acid-modifying enzyme, and a further nucleic acid molecule that is ligated to the nucleic acid substrate in the presence of the nucleic acid-modifying enzyme to a ligation product; and

(b) assessing the effect of the agent on the amount of ligation product produced.

Thus, the invention provides a method of assessing whether an agent has an effect on a nucleic acid-modifying enzyme (e.g. a Mycobacterium type I topoisomerase, a Plasmodium type I topoisomerase, a Streptococcus type I topoisomerase, or a retroviral integrase such as HIV, SIV, FIV or MLV integrase) in a sample, the method comprising:

(a) providing a sample;

(b) providing a nucleic acid substrate targeted by the nucleic acid-modifying enzyme;

(c) providing a further nucleic acid molecule that is ligated to the nucleic acid substrate in the presence of the nucleic acid-modifying enzyme;

(d) providing an agent

(d) combining the sample of step (a), the nucleic acid substrate of step (b) and the further nucleic acid molecule of step (c), which combination is done with or without the agent of step (d); and

(e) detecting the ligation product produced in step (d), with or without the agent.

Preferences for the sample, nucleic acid substrate, nucleic acid-modifying enzyme, and further nucleic acid include those described above in relation to the first aspect of the invention. Preferably the ligation product is detected using the SHOfLE oligonucleotides defined herein. As described above, the further nucleic acid molecule may be a part of the nucleic acid substrate.

By effect on the activity of a nucleic acid-modifying enzyme, we include the meaning of both increasing and decreasing the activity of the nucleic acid-modifying enzyme. For example, if the agent increases the amount of ligation product produced, the agent is expected to be an activator of the enzyme (i.e. one that increases its activity), and if the agent decreases the amount of ligation product produced, the agent is expected to be an inhibitor of the enzyme (i.e. one that decreases its activity).

The nucleic acid-modifying enzyme in the sample (e.g. a type I topoisomerase) may be one that is contained in a cell expressing it, for example a cancer cell. Thus, it will be appreciated that this method may be useful to identify potential therapeutic agents that inhibit the growth and/or survival of cancer cells (e.g. cancer cells expressing a particular nucleic acid-modifying enzyme (e.g. a type I topoisomerase) or having a particular activity of nucleic acid-modifying enzyme (e.g. a type I topoisomerase), wherein the enzyme or its activity is unique to the cancer cells).

As in the fourth aspect of the invention, it will be appreciated that it may be desirable to assess the amount of ligation product formed in the absence of the agent, to provide a baseline level to which the amount of ligation product formed in the presence of the agent can be compared to. However, it will also be appreciated that the baseline amount of ligation product may already be known, and so there is no need to perform another assay to calculate it.

As with the fourth aspect of the invention, the method of assessing whether an agent has an effect on the activity of a nucleic acid-modifying enzyme includes screening methods to identify drugs or lead compounds for use in treating a disease or condition mediated by aberrant expression of a nucleic acid-modifying enzyme (e.g. overexpression or underexpression of a nucleic acid-modifying enzyme or expression of a nucleic acid-modifying enzyme in an aberrant cell (e.g. cell or tissue)). It is appreciated that screening assays which are capable of high throughput operation are particularly preferred.

Also, the method may further comprises modifying an agent which has been shown to have an effect on the activity of a nucleic acid-modifying enzyme (e.g. an inhibitory effect), and testing whether the modified agent has an effect on the activity.

Once an agent or modified agent which has an effect on the activity of a nucleic acid-modifying enzyme (e.g. an inhibitory effect), has been found, it may be desirable to test its effect in a suitable model in vivo. For example, the method may comprise testing the ability of the agent or modified agent to affect the activity of the nucleic acid-modifying enzyme in a sample taken from an in vivo model of a disease or condition mediated by aberrant expression of a the nucleic acid-modifying enzyme. For example, the effect of the agent or modified agent may be tested in an animal model of cancer, wherein the cancer cells express a particular nucleic acid-modifying enzyme (e.g. a type I topoisomerase) or have a particular activity of nucleic acid-modifying enzyme (e.g. a type I topoisomerase) and the enzyme or its activity is unique to the cancer cells.

The method may further comprise determining whether the agent or modified agent modulates or affects disease severity, duration or progression in the in vivo model of the disease or condition (e.g. cancer).

In a further embodiment, the method may also comprise the step of formulating the agent which has an effect on the activity of a nucleic acid-modifying enzyme (e.g. an inhibitory effect) such as a type I topoisomerase, into a pharmaceutically acceptable composition.

Therefore, the invention also includes a pharmaceutical composition comprising an agent which has an effect on the activity of a nucleic acid-modifying enzyme, such as a type I topoisomerase (e.g. an inhibitory effect) that has been identified as described above, and a pharmaceutically acceptable carrier, diluent or excipient.

A sixth aspect of the invention provides a device for carrying out a method according to the first or fifth aspects of the invention.

In one embodiment, the device comprises a means for washing the nucleic acid substrate immobilised on the surface, for example according to step (a)(i) of the method of the first or fifth aspects of the invention. For example, the device may contain an inlet channel through which a wash buffer can be transferred to a chamber containing the nucleic acid immobilised on the surface, and an outlet channel through which the wash buffer may be removed from the chamber. By passing or pumping wash buffer both through the inlet channel and out of the outlet channel, the nucleic acid substrate may be washed as described in the first or fifth aspects of the invention. Thus, it will be appreciated that the device may contain a pump for passing wash buffer over the surface to which the nucleic acid substrate is immobilised.

The device may comprise separate chambers for the different reaction or steps of the methods of the invention. For example, the device may comprise a separate processing chamber, comprising the nucleic acid substrate and further nucleic acid molecule, and an amplification chamber comprising the one or more detectable oligonucleotides that bind to the linear single strand of nucleic acid on the ligation product (e.g. SHOfLE oligonucleotides), and a detection chamber comprising means for detection of the amplified product (e.g. assembly of SHOfLE oligonucleotides), for example a fluorescent microscope. The device may comprise further chambers, or the chambers may be merged into fewer chambers. For example, in one embodiment, the device comprises a separate reaction chamber and detection chamber. By reaction chamber, we include the meaning of the chamber wherein the nucleic acid substrate and the further nucleic acid molecule are ligated by the nucleic acid-modifying enzyme in the sample to form the ligation product. By detection chamber, we include the meaning of the chamber wherein the ligation product is detected. Thus, the reaction chamber may contain the nucleic acid substrate and further nucleic acid molecule, and the'detection chamber may include the one of more detectable oligonucleotides that bind to the linear single strand of nucleic acid on the ligation product (e.g. SHOfLE oligonucleotides). Preferably, the nucleic acid substrate within the device is immobilised on a surface as described above in more detail in relation to the first aspect of the invention.

In one embodiment, the device comprises a control chamber. By control chamber we include the meaning of a chamber where a negative control and/or a positive control can be performed, optionally in parallel with the detection of the nucleic acid-modifying enzyme of the microorganism, cell or cell type. A negative control may comprise contacting the nucleic acid substrate and the further nucleic acid product in the absence of sample but otherwise under the same reaction conditions as the nucleic acid substrate and further nucleic acid molecule in the presence of the sample. A positive control may comprise contacting the nucleic acid substrate and the further nucleic acid product in the presence of the nucleic acid-modifying enzyme that selectively targets the nucleic acid substrate, and under the same reaction conditions as the nucleic acid substrate and further nucleic acid molecule in the presence of the sample. It will be understood that the controls allow for the verification of substrate function, enzyme activity etc.

Where the device contains separate chambers for different reactions, the device may further comprise means for transferring the ligation product from the reaction to the detection chamber. In one embodiment, the device comprises a magnet for transferring the ligation product and/or the one or more detectable oligonucleotides (e.g. SHOfLE oligonucleotides). In such a device, the ligation product and/or one or more detectable oligonucleotides are preferably coupled to magnetic beads or the like, thereby allowing the use of the included magnet for transferring the ligation product and/or detectable oligonucleotides from one compartment, such as a reaction chamber or amplification chamber, to a detection chamber.

It will be appreciated that the device may allow for multiplexed analysis, wherein two or more microorganisms, cells or cell types are detected in parallel or simultaneously. Such devices will comprise a plurality of nucleic acid substrates, each targeted by a different nucleic acid-modifying enzyme (e.g. type I topoisomerase).

Conveniently, the device comprises a waste outlet. For example, this may allow dispensing of spent wash buffer.

The device may comprise a means that allows the ligation product to be detected by any of the techniques described above in relation to the first aspect of the invention. Examples of such means are means for southern blotting, PCR, RT-PCR, qPCR, RFLD, primer extension, DNA array technology, isothermal amplification, and/or rolling circle amplification. Preferably, the means are means for detecting assemblies of SHOfLE oligonucleotides defined herein.

In a further embodiment, the device further comprises one or more analytical means to detect the ligation product. For example, the device may comprise a means to detect and/or quantify the detectable signal produced when the ligation product is detected by one of the techniques described herein, such as the use of SHOfLE oligonucleotides (e.g. a radioactive signal or a fluorescent signal or a chemiluminescent signal or a colometric signal). Suitable means are known in the art and include a photometer or a fluorometer or a radiometer or imaging apparatus.

The device may be a microfluidic system whereby the methods of the invention can be at least partly implemented in a microfluidic setup. Microfluidic systems are described in WO2013029631, which is herein incorporated by reference, and particularly pages 23-27 thereof. It will be appreciated that the device may comprise any one or more of the features described therein that enable the microfluidic setup. For example, the device may comprise a sample chamber that comprises one or more inlet channels for loading components into the microfluidic system, and an outlet channel that may be formed as a serpentine channel that serves for the components of the droplets to be adequately mixed. From the sample chamber, droplets are generated and transferred via an outlet flow through channel to a droplet retaining means, where one or more single droplets are captured in individual cavities and each single droplet is spatially isolated from other droplets.

A seventh aspect of the invention provides a kit of parts comprising a nucleic acid substrate targeted by a nucleic acid-modifying enzyme and a means for detecting a ligation product formed by ligation of the nucleic acid substrate to a further nucleic acid molecule, which ligation product comprises a linear single strand of nucleic acid that is capable of being detected.

It will be appreciated that the device of the sixth aspect of the invention and kit of parts of the seventh aspect of the invention, may be used to carry out the methods of the earlier aspects of the invention. Thus, the nucleic acid-modifying enzyme may be one from a microorganism, cell or cell type, and the nucleic acid substrate may be one that is targeted by the nucleic acid-modifying enzyme from that microorganism, cell or cell type. Examples of possible microorganisms, cells and cell types include those described above. Preferably, the microorganism is a Plasmodium (e.g. Plasmodium falciparum) or the microorganism is a Mycobacterium (e.g. mycobacterium tuberculosis) or the microorganism is a retrovirus (e.g. HIV, SIV, FIV or MLV) or the microorganism is a Streptococcus (e.g. Group B Streptococcus such as Streptococcus agalactiae). Preferably, the cell or cell type is a cancer cell.

In an embodiment, the kit of parts comprises a further nucleic acid molecule. The further nucleic acid molecule may be any nucleic acid molecule as defined herein provided that it can be ligated to the nucleic acid substrate in the presence of the nucleic acid-modifying enzyme to form a ligation product that comprises a linear single stand of nucleic acid that is capable of being detected. Typically, the further nucleic acid molecule is double stranded DNA or single stranded DNA. The further nucleic acid molecule may be part of the nucleic acid substrate.

Preferences for the nucleic acid substrate, nucleic acid-modifying enzyme, further nucleic acid molecule, ligation product, and means for detecting the ligation product, include those defined above in relation to the first aspect of the invention.

For example, the nucleic acid substrate may be one that is selectively targeted by a Plasmodium type I topoisomerase (e.g. a double stranded DNA wherein at least one strand comprises a sequence selected from the group consisting of ACTACCATTCTGAGTCGTTCGAAGTTCCTATACTTT (SEQ ID No: 1) and TCTAGAAAGTATAGGAACTTCGAACGACTCAGAATG (SEQ ID No: 2), or a double stranded DNA wherein at least one strand comprises a sequence selected from the group consisting of ACTACCATTCTGAGTCGTTCGATCTAAAAGACTTAGA (SEQ ID No: 3) and ATTTTTCTAAGTCTTTTAGATCGAACGACTCAGAATG (SEQ ID No: 4), any of which sequences may be varied as outlined above), and the nucleic acid-modifying enzyme may be a Plasmodium type I topoisomerase.

In another example, the nucleic acid substrate may be one that is selectively targeted by a Mycobacterium type I topoisomerase (e.g. a single stranded DNA that comprises the sequence CAGTGAGCGAGCTTCCGCTTGACATCCCAATAGTTTCTCTTC (SEQ ID No: 5) which sequence may be varied as outlined above), and the nucleic acid-modifying enzyme may be a Mycobacterium type I topoisomerase.

In yet another example, the nucleic acid substrate may be one that is selectively targeted by an integrase, such as a HIV integrase (e.g. a double stranded DNA wherein at least one strand comprises a sequence selected from the group consisting of TTTAGTCAGTGTGGAAAATCTCTAGCAGT (SEQ ID No: 6) and ACTGCTAGAGATTTTCCACACTGACTAAA (SEQ ID No: 7) either of which sequences may be varied as outlined above) or an MLV integrase (e.g. a double stranded DNA wherein at least one strand comprises a sequence selected from the group consisting of TTGACTACCCGTCAGCGGGGGTCTTTCATT (SEQ ID No: 22) and AATGAAAGACCCCCGCTGACGGGTAGTCAA (SEQ ID No: 23) either of which sequences may be varied as outlined above), and the nucleic acid-modifying enzyme may be an integrase (e.g. a HIV integrase or a MLV integrase).

In still another example, the nucleic acid substrate may be one that is selectively targeted by a Streptococcus type I topoisomerase, such as Group B Streptococcus (e.g. Streptococcus agalactiae) type I topoisomerase (e.g. single stranded DNA that comprises the sequence ACTTCGGGATGCTGAAACGCAGCTGTGC (SEQ ID No: 28) which sequence may be varied as outlined above), and the nucleic acid-modifying enzyme may be a Streptococcus type I topoisomerase (e.g. Group B Streptococcus (e.g. Streptococcus agalactiae)).

In an embodiment, the means for detecting the ligation product'comprises one or more detectable oligonucleotides as described above in relation to the first aspect of the invention. It is preferred if the one or more detectable oligonucleotides are SHOfLE oligonucleotides as defined herein. Conveniently, the means are preferably coupled to magnetic beads or the like, thereby allowing the use of a magnet for transferring the ligation product and/or detectable oligonucleotides from one compartment to another in the device described above. However, other means are also encompassed by the invention, such as means for southern blotting, polymerase chain reaction, RT-PCR, qPCR, RFLD, primer extension, DNA array technology, isothermal amplification, and/or rolling circle amplification.

The kit may be presented in any appropriate physical form, which allows for easy use thereof. In a preferred embodiment, the kit is provided wherein the nucleic acid substrate is immobilised on a solid support, optionally wherein the further nucleic acid molecule is immobilised on a solid support. Suitable supports are described above in relation to the first aspect of the invention and include glass, plastic, metal, silicon, ceramics and latex. Thus, the kit may be provided as a lateral flow test strip and/or a dipstick. The lateral flow test may be provided in a dipstick format. Lateral flow tests are simple devices for detecting the presence (or absence) of a target analyte in a sample, which in the present invention is the ligation product. In the lateral flow test, the sample flows along a solid substrate, preferably by capillary action. After the sample is applied to the test, it first encounters one or more nucleic acid substrates and further nucleic acid molecules immobilised on the test strip. The substrate and further nucleic acid molecule are here targeted by the nucleic acid-modifying enzyme (e.g. type I topoisomerase or integrase) of the sample and ligated. Then, the ligation product flows or is actively transferred to encounter detectable entities that bind to the linear single strand of nucleic acid on the ligation product. These entities are preferably one or more detectable oligonucleotides such as SHOfLE oligonucleotides defined herein, in which case the SHOfLE oligonucleotides form an assembly which is then visualised, for example by silver staining or fluoroscopy. The one or more detectable oligonucleotides (e.g. SHOfLE oligonucleotides) may be conveniently labelled with one or more fluorescent or other dyes, radioactive nucleotides, one or more moieties that allow detection by silver staining, and/or biotinylated nucleotides.

In an embodiment, the kit of parts does not include an oligonucleotide primer (e.g. a PCR primer). In a further embodiment, the kit of parts does not include a polymerase enzyme. In yet a further embodiment, the kit of parts does not include an oligonucleotide primer and does not include a polymerase enzyme.

In an embodiment, the kit comprises a plurality of nucleic acid substrates each targeted by a nucleic acid-modifying enzyme of a different microorganism, cell or cell type. In this way, the kit allows the detection of multiple microorganisms, cells or cell types. By nucleic acid-modifying enzyme of a different microorganism, cell or cell-type, we include the meaning of the respective nucleic acid substrates being selectively targeted by different respective nucleic acid-modifying enzymes which derive from different microorganisms, cells or cell types. For example, the kit may comprise a nucleic acid substrate that is selectively targeted by a Plasmodium type I topoisomerase (e.g. Plasmodium falciparum type I topoisomerase), and/or a nucleic acid substrate that is selectively targeted by a Mycobacterium type I topoisomerase (e.g. Mycobacterium tuberculosis type I topoisomerase) and/or a nucleic acid substrate that selectively targeted by a Streptococcus type I topoisomerase (e.g. Group B Streptococcus type I topoisomerase). Alternatively, the kit may comprise a nucleic acid substrate that is selectively targeted by a type I topoisomerase from a particular cancer cell, and a nucleic acid substrate that is selectively targeted by a particular non-cancer cell. In each case, it will be understood that the nucleic acid substrate is preferably one that is selectively targeted by a nucleic acid-modifying enzyme of a given microorganism, cell or cell type, and most preferably is not targeted by a nucleic acid-modifying enzyme of another microorganism, cell or cell type.

The kit of parts may include reagents to carry out one or more control reactions (e.g. positive and/or negative controls) alongside the test reaction, as described above in relation to the sixth aspect of the invention. For example, as a positive control, the kit of parts may further comprise the nucleic acid-modifying enzyme that selectively targets the nucleic acid substrate of the kit.

As described above, a useful way of minimising false positives in the methods of the invention is by depleting the sample and/or reaction buffers of entities that are required for the activity of other nucleic acid-modifying enzymes that may be present in the sample being tested, but which are not required for the activity of the nucleic acid-modifying enzyme for which it is desired to detect. For example, the sample or reaction buffers may be depleted of divalent cations, which are a prerequisite for the activity of most nucleic acid-modifying enzymes, including ligases, but not for type I topoisomerases such as Plasmodium falciparum type I topoisomerase and Mycobacterium tuberculosis type I topoisomerase. Thus, the kit of parts may further comprise an agent for depletion of divalent cations. Examples of such agents include a chelating agent, such as EDTA, or any other effective agent.

The invention will now be described with the aid of the following Figures and Examples.

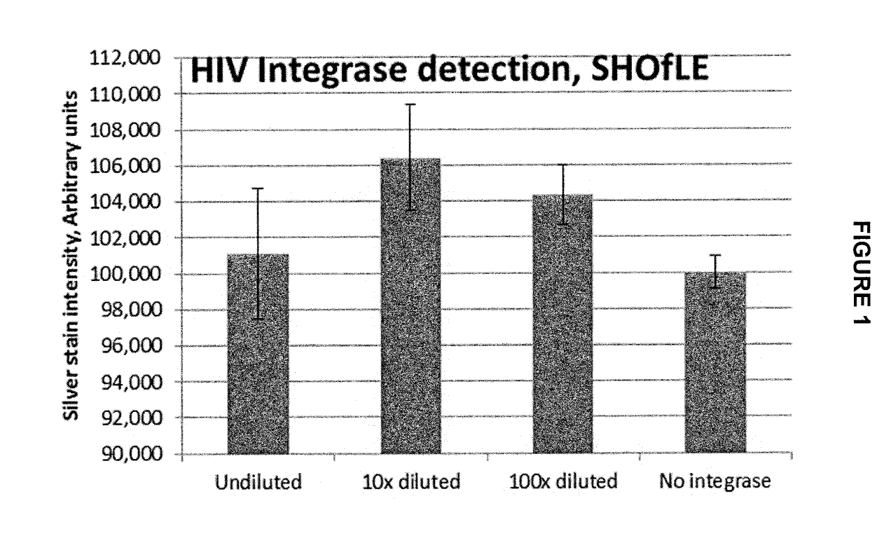

FIG. 1: HIV integrase detection by SHOfLE methodology (see Example 3). SHOfLE was performed as described in the text using purified HIV integrase. The integrase was diluted in storage buffer (200 mM KCl, 10 μM ZnCl₂, 50% glycerol) as indicated on the figure. The “no integrase control” was just added storage buffer. The reaction was carried out in retrovirus reaction buffer supplemented with a double stranded DNA substrate (the further nucleic acid) and 10% (v/v) purified undiluted or diluted integrase.

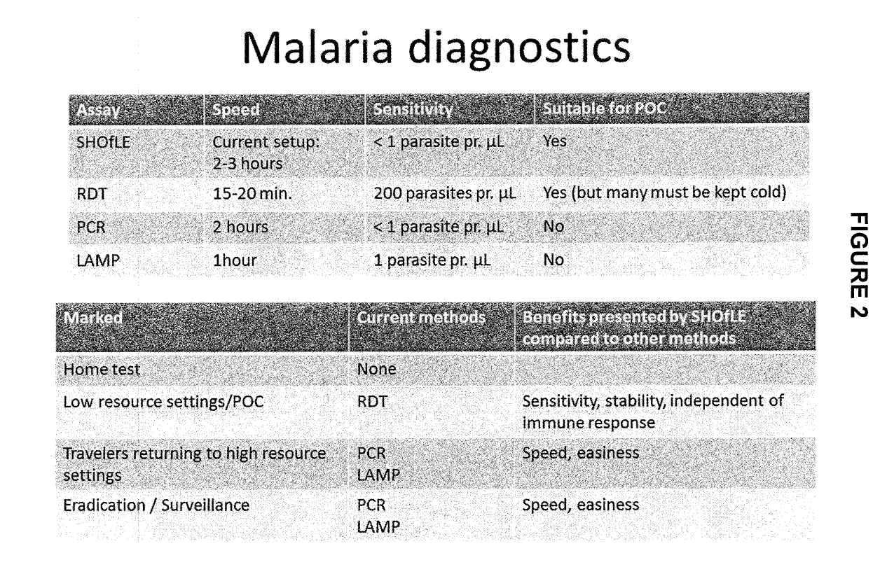

FIG. 2: Comparison of the SHOfLE strategy to current diagnostic methods

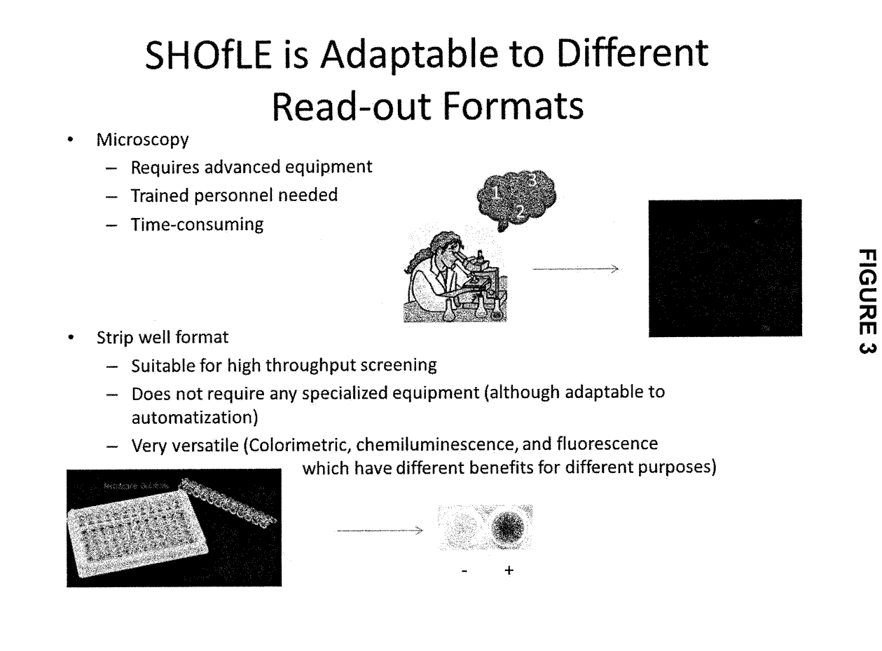

FIG. 3: Comparison of the microscopy based readout to the strip well based read out.

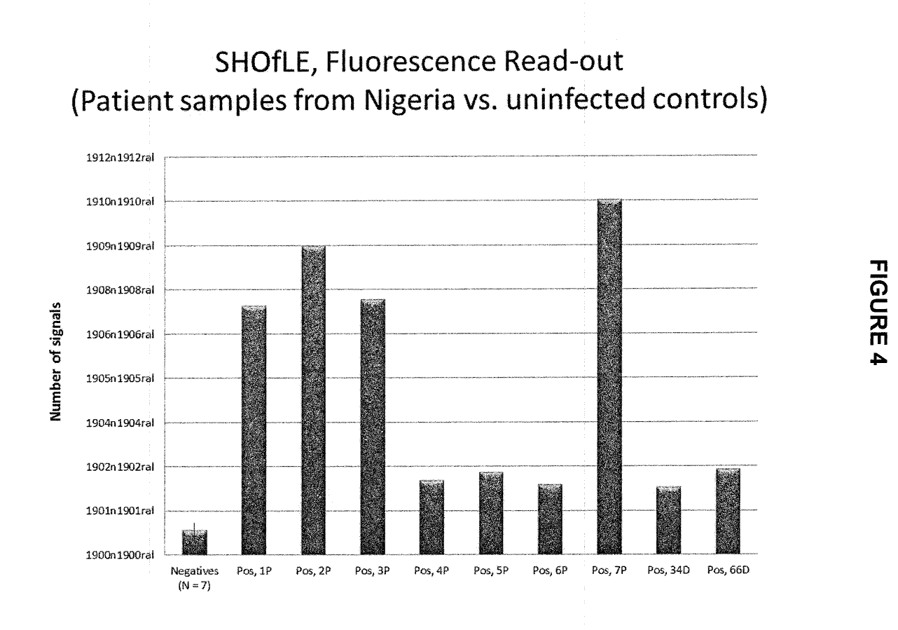

FIG. 4: Detection of the malaria parasite (via pfTopol) in blood samples from malaria infected patients. The blood samples had only been frozen once at the time of analysis. The blood samples were lysed as described and subjected to SHOfLE. The infected samples were compared to blood samples from seven uninfected individuals. One of the SHOfLE oligonucleotides was labeled with Fam and the products were hence visible by fluorescence microscopy.

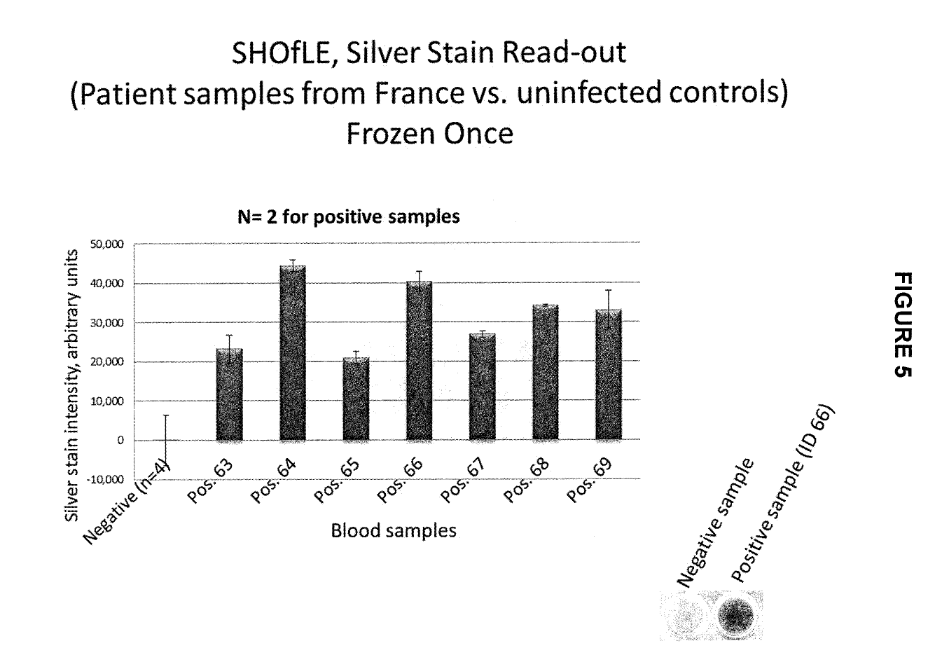

FIG. 5: Detection of the malaria parasite (via pfTopol) in blood samples from malaria infected patients. The blood samples had only been frozen once at the time of analysis. The blood samples were lysed as described and subjected to SHOfLE. The infected samples were compared to blood samples from four uninfected individuals. One of the SHOfLE oligonucleotides was labeled with biotin and the products was visible by binding to streptavidin conjugated gold nanoparticles and subsequent silver precipitation on the gold nanoparticles.

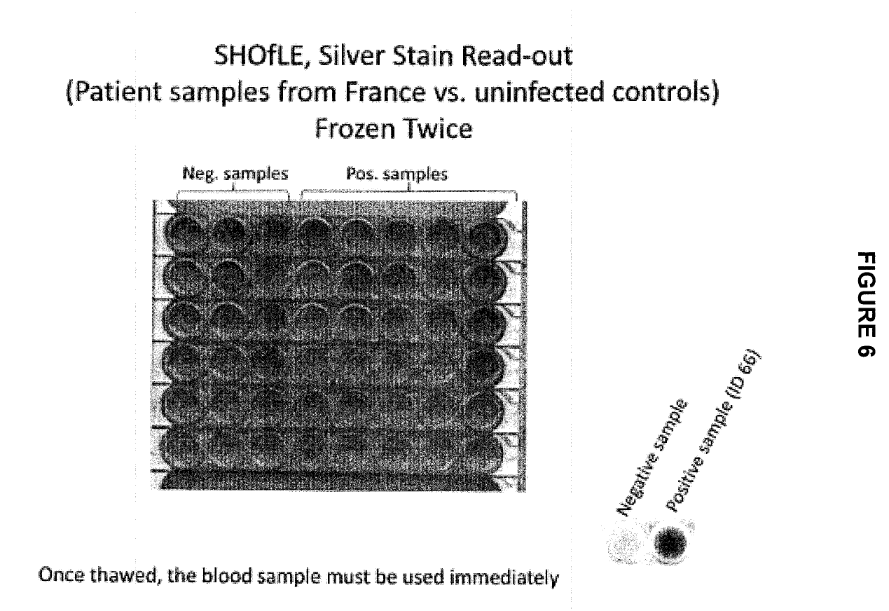

FIG. 6: Detection of the malaria parasite (via pfTopol) in blood samples from 30 malaria infected patients. The blood samples had been frozen twice at the time of analysis. The blood samples were lysed as described and subjected to SHOfLE. The infected samples were compared to blood samples from 18 uninfected individuals. One of the SHOfLE oligonucleotides was labeled with biotin and the products was visible by binding to streptavidin conjugated gold nanoparticles and subsequent silver precipitation on the gold nanoparticles.

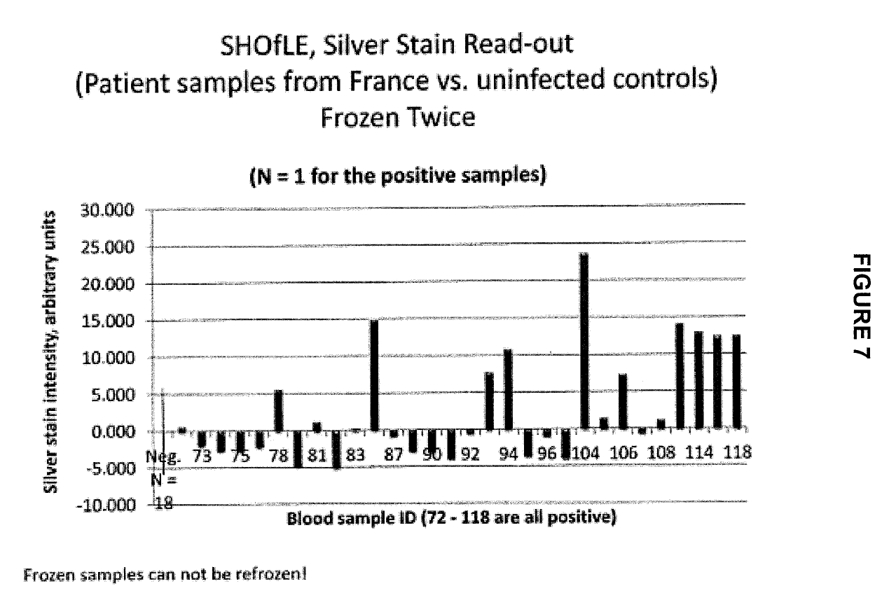

FIG. 7: Graph showing quantification of the data in FIG. 6.

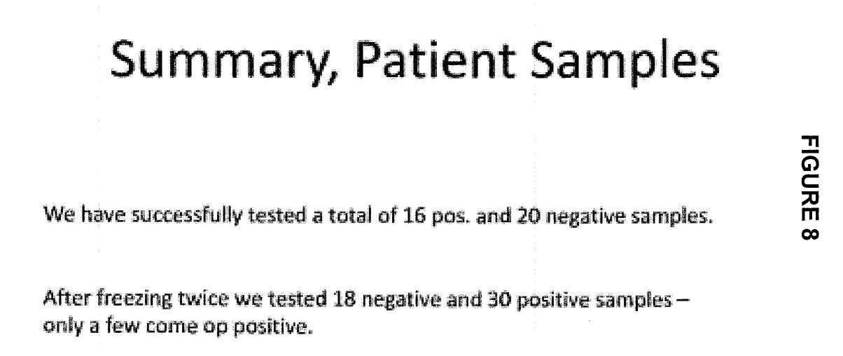

FIG. 8: Summary of conclusions.

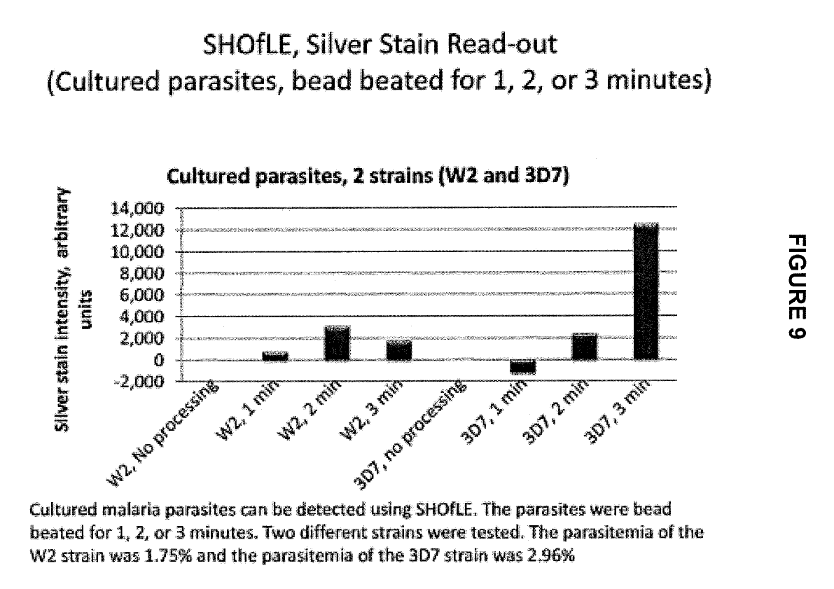

FIG. 9: Cultured parasites was lysed by mixing 80 μL of blood with 320 μL TE (10 mM Tris-HCl, pH 7,5 and 1 mM EDTA). This mixture was subjected to bead beating for 1, 2, or 3 minutes. The lysate was analyzed by SHOfLE as described for vortexed samples.

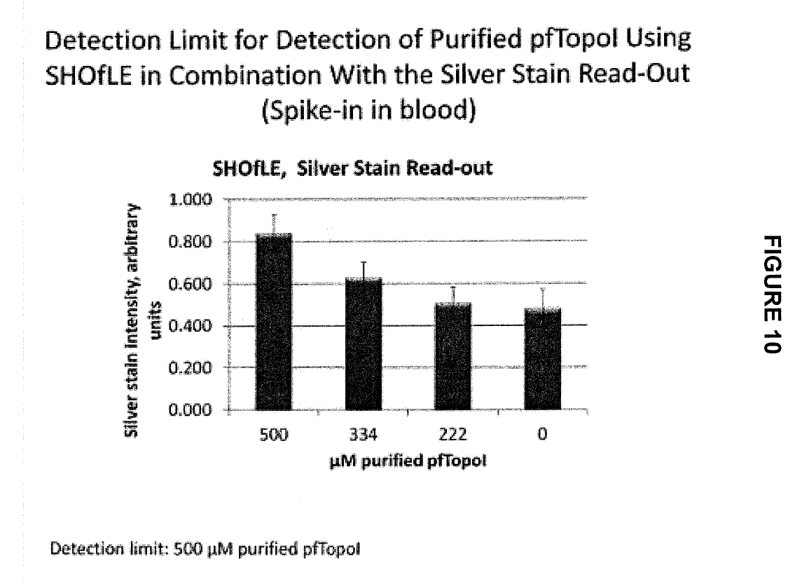

FIG. 10: SHOfLE detection (silver stain) of different amounts of purified pfTopol.

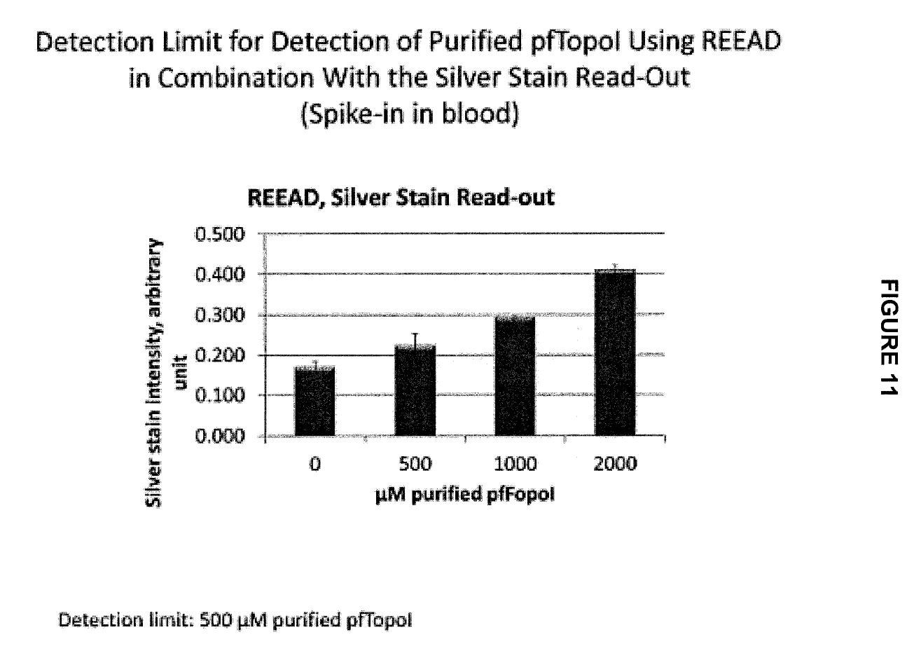

FIG. 11: REEAD detection (incorporation of biotin during RCA, silverstain as for SHOfLE) of different amounts of purified pfTopol.

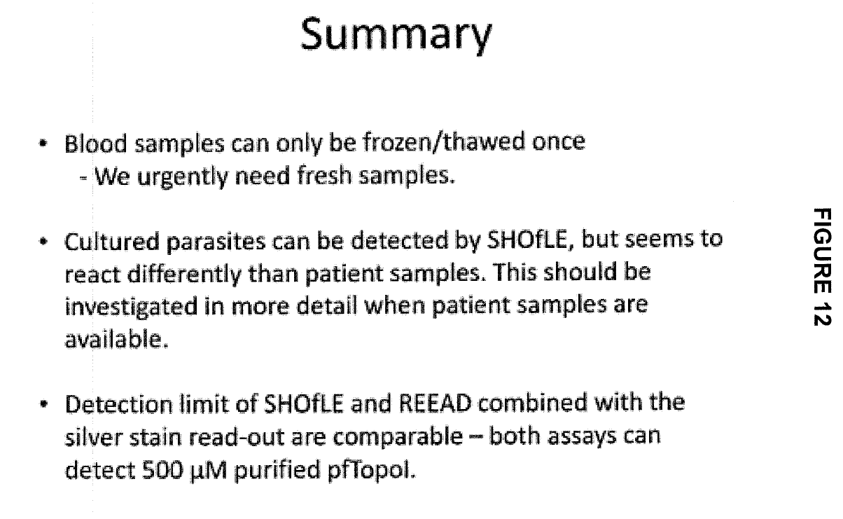

FIG. 12: Summary of conclusions.

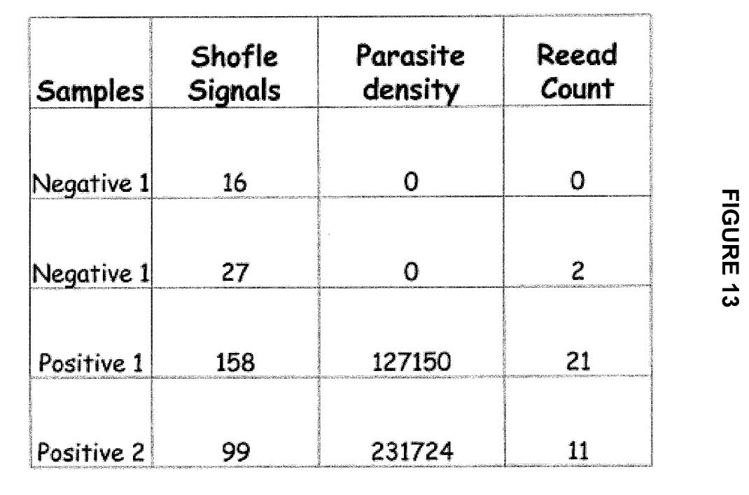

FIG. 13: Comparison of SHOfLE and REEAD assays.

FIG. 14: Schematic depiction of the SHOfLE strategy.

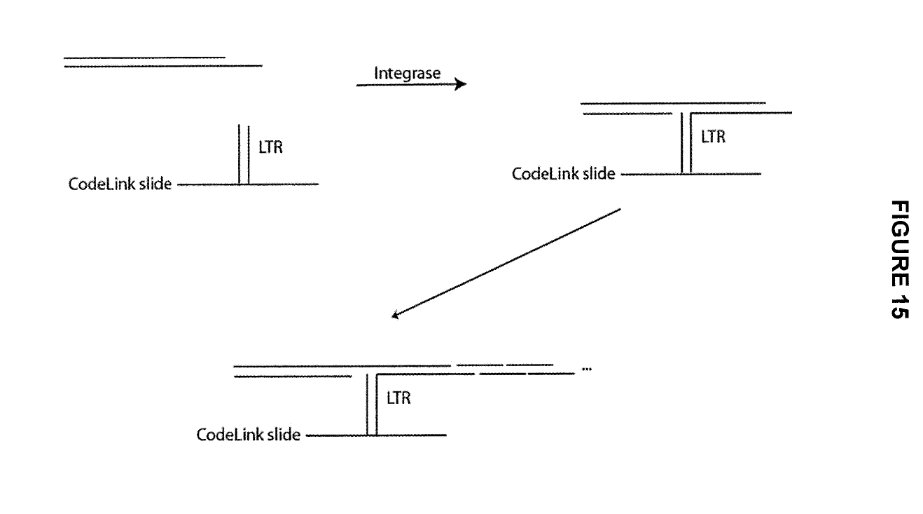

FIG. 15: Schematic depiction of retrovirus detection by SHOfLE

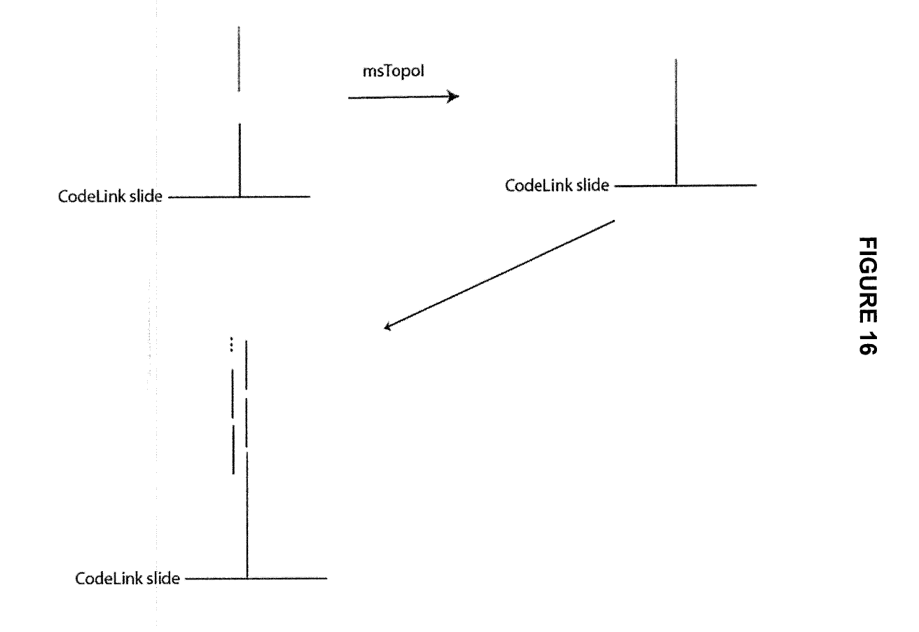

FIG. 16: Schematic depiction of msTopol (or other mycobacterial type 1A topoisomerase) SHOfLE.

FIG. 17: Schematic depiction of detection of nucleic acid-modifying enzyme where ligation product is formed indirectly and further nucleic acid molecule is part of nucleic acid substrate. (A) The nucleic acid substrate comprises two strands, a first strand immobilised to a bead and a second strand hybridised to the first strand but not immobilised to a bead. This second strand corresponds to the further nucleic acid molecule which also comprises a double stranded nucleic acid portion separate from the portion of the further nucleic acid molecule that is hybridised to the first strand of the nucleic acid substrate. The double stranded nucleic acid portion of the further nucleic acid molecule may comprise one or more detectable oligonucleotides, denoted in the figure as stars. In the presence of a nucleic acid-modifying enzyme (target), the first and second strands of the nucleic acid substrate are ligated. The nucleic acid substrate is then washed under denaturing conditions. If the two strands of the nucleic acid substrate have been ligated, then exposure to denaturing conditions results in the formation of a ligation product that comprises a linear single strand of nucleic acid that is capable of being detected. If, as shown in the figure, this linear single strand of nucleic acid comprises one or more detectable oligonucleotides, then the presence of a signal following the wash step is a direct read-out of the present of nucleic acid-modifying enzyme. If the two strands of the nucleic acid substrate have not been ligated, then exposure to denaturing conditions does not result in formation of the ligation product and no signal is detected because the further nucleic acid molecule is separated from the first strand of nucleic acid substrate that remains immobilised on the surface. (B) A more specific embodiment than that shown in (A) where the double stranded nucleic acid portion of the further nucleic acid molecule comprises one or more fluorescently labelled oligonucleotides.

FIG. 18: PFTopol mediated ligation of a PCT product to a surface. Ligation product comprising linear single strand of nucleic acid is formed either directly (A) or indirectly (B). In (B), the nucleic acid substrate comprises two strands as it does in FIG. 17, and the further nucleic acid molecule is part of this nucleic acid substrate. A first strand of the nucleic acid substrate is immobilised to a codelink slide and a second strand is hybridised to the first strand but is not immobilised to the codelink slide.

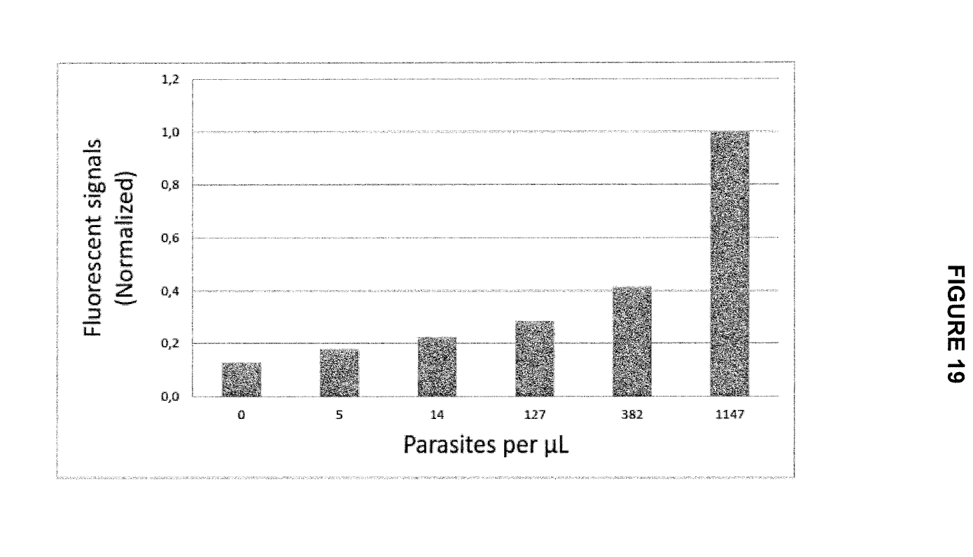

FIG. 19: Detection of pfTopo I in blood lysate.

### EXAMPLE 1

### Use of SHOfLE Technology to Detect Plasmodium falciparum Topoisomerase I

Methodology

Cloning, Expression, and Purification of pfTopo1

The gene encoding plasmodium falciparum topoisomerase I (pfTopol) was cloned into the pYES2.1 vector from Invitrogen using the pYES2.1 TOPO TA Expression Kit. The resulting vector enables galactose inducible expression of pfTopol in yeast. Purification of pfTopo1 was done essentially as described previously (Lisby et al, J Biol Chem. 2001 June 8; 276(23):20220-7). Purified enzyme was stored at −20° C.

Blood Sample Preparation

8 μL of fresh blood sample or a blood sample only frozen once is mixed with 32 μL of hypotonic lysis buffer (10 mM Tris-HCL pH 7.5; 5 mM EDTA, 0.2% Tween 20; 1 mM DTT, 1 mM PMSF) and 100 μL of Pico-Surf (TM) 1 (2% in Novec 7500). Droplets are generated by vortexing the samples for one minute. The droplets are incubated on ice for 15 minutes and broken by addition of 15 μL 1H,1H,2H,2H-Perfluoro-1-octanol.

Coupling of Amine Labelled Substrate to Microtiter Wells

The substrate for pfTopol is a DNA duplex consisting of two amine coupled 36-meric oligonucleotides. To immobilize the substrate on Well-Coated™ Amine Binding, 8-well strip plates from G-biosciences, we first couple one strand covalently to the wells and then hybridize the other strand to the covalently attached oligonucleotide.

Covalent Coupling of Amine Conjugated Oligonucleotide to Microtiter Wells

The amine-couple oligonucleotide ([AmC6T]actaccattctgagtcgttcgaagttcctatacttt; SEQ ID No: 1) is diluted in 50 mM sodium-phosphate buffer, pH 8.5 to a final concentration of 5 μM. 50 μL of this solution is added to each amine binding well.

The wells are incubated over night at room temperature in a humidity chamber containing a saturated NaCl solution.

Next day, the wells are processed as follows: 30 minutes in warm (50° C.) blocking buffer, 2×1 minute wash in water, 30 minutes in warm (50° C.) wash buffer 1, 2×1 minute wash in water. Let the wells air-dry.

Hybridization of Second Substrate Strand to the Covalently Attached Strand

The second substrate strand ([AmC6T]TCTAGAAAGTATAGGAACTTCGAACGACTCAGAATG; SEQ ID No: 2) is diluted to a final concentration of 0.2 μM in a buffer containing 10 mM Tris (pH 7.5), 1 mM EDTA, and 300 mM NaCl. 50 μL of this solution is added to each well. Incubate at 37° C. for 1 h in humidity chamber. Wash for 1 min in wash buffer 2, 1 min in wash buffer 3, and 1 min in 96% ethanol. Let the wells air-dry.

Topoisomerase I Reaction

Prepare a solution containing 20% (v/v) broken droplets (see “Blood sample preparation”) or 10% (v/v) purified pfTopol (different concentrations as indicated on figure) as well as 10 mM Tris-HCl (pH 7.5), 1 mM EDTA, and 300 mM NaCl. Add 50 μL to the each microtiter well. Incubate for 1 h at 37° C. in humidity chamber. Wash 3×5 minutes in wash buffer 4. To each well, add 50 μL of Ligation mixture: 2 μM linker oligo (TTCTAGACCAGACGCTCACTGAGCGGAAGCTTCAG; SEQ ID No: 15); 300 mM NaCl; 1 mM Tris-HCl (pH 7.5); 5 mM MgCl₂. Incubate at 37° C. for 1 h in humidity chamber. Wash for 1 min in wash buffer 2, 1 min in wash buffer 3, and 1 min in 96% ethanol. Let the wells air-dry.

SHOfLE

Add 50 μL of SHOfLE mixture to each well: 2×SSC, 20% formamid, 5% glycerol, and 0.2 μM of each of the biotin labelled SHOfLE oligoes ([Btn]CAG TGA GCG TCT GGC TGA AGC TTC CGC T; SEQ ID No: 16; [Btn]CCA GAC GCT CAC TGA GCG GAA GCT TCA G; SEQ ID No: 17). Incubate at 50° C. for 3 h in humidity chamber. Wash for 20 min in wash buffer 2, 10 min in wash buffer 3 and 1 min in 96% ethanol. Let the wells air-dry.

Silver Staining

Strep-Gold reaction mix: 2% (v/v) streptavidin coupled gold nanoparticles (Nanocs; cat. no. GNA3; 0.05% w/v), 0.5% BSA in 0.1 M PBS, pH 10+10 μl of 5% BSA. Add 50 μL to each well and incubate at room temperature for 15 minutes. Wash for 5 minutes in wash buffer 2, wash for 5 minutes in wash buffer 3, rinse in water, and wash for 1 min in 96% ethanol. Let the wells air-dry.

Solution A and Solution B from the silver enhancer kit (SigmaAldrich, SE100) are mixed 1:1 immediately before use and applied to the wells. Develop for 10 minutes. After development, the wells are rinsed in water and dried. The amount of silver precipitated is quantified using a POLARstar Omega fluorescence reader.

Buffer

Blocking buffer: 50 mM Ethanolamine, 0.1 M Tris (pH 9.0)

Wash buffer 1: 4×SSC; 0.1% SDS

Wash buffer 2: 0.1M Tris-HCl (pH 7.5); 150 mM NaCl; 0.3% SDS

Wash buffer 3: 0.1M Tris-HCl (pH 7.5); 150 mM NaCl; 0.05% Tween 20

Wash buffer 4: 10 mM Tris-HCl (pH 7.5), 1 mM EDTA, 1M NaCl

### EXAMPLE 2

### Use of SHOfLE Technology to Detect Mycobacterial Topoisomerase I

Summary

As a model system for mycobacterial topoisomerase I, we use topoisomerase I from Mycobacterium smegmatis (msTopol). To enable SHOfLE detection of msTopol, we immobilize a single stranded substrate for msTopol. msTopol can mediate ligation of one of the SHOfLE oligonucleotides to this substrate strand and thereby create a base for SHOfLE hybridization.

Methodology

Coupling of msTopol Substrate to an Amine Binding Surface (CodeLink Slide)

The msTopol substrate 5′-CAG TGA GCG AGC TTC CGC TTG ACA TCC CAA TAG TTT CTC TTC-Amino-C6-3 (SEQ ID No: 5) is coupled to CodeLink slides (SurModics) as described below: The amine-couple oligonucleotide is diluted in 50 mM sodium-phosphate buffer, pH 8.5 to a final concentration of 5 μM. 1 μL of this solution is added to a small area of a CodeLink slide (marked using a hydrophobic PAP pen). The oligonucleotide conjugated area of the CodeLink slide is denominated the printed area.

The slide is incubated over night at room temperature in a humidity chamber with saturated salt.

Next day, the slide is processed as follows: 30 minutes in warm (50° C.) blocking buffer, 2×1 minute wash in water, 30 minutes in warm (50° C.) wash buffer 1, 2×1 minute wash in water. Let the slide air-dry.

msTopo Binding Reaction

Prepare a solution containing msTopol as well as 10 mM Tris-HCl (pH 7.5), 1 mM EDTA, and 300 mM NaCl. Add 5 μL to each printed area (see previous section). Incubate for 1 h at 37° C. in humidity chamber. Wash 3×5 minutes in wash buffer 4. To each printed are, add 5 μL of ligation mixture: 2 μM oligonucleotide ([Btn]CAG TGA GCG TCT GGC TGA AGC TTC CGC T; SEQ ID No: 16); 300 mM NaCl; 1 mM Tris-HCl (pH 7.5); 5 mM MgCl₂. Incubate at 37° C. for 1 h in humidity chamber. Wash for 1 min in wash buffer 2, 1 min in wash buffer 3, and 1 min in 96% ethanol. Let the slide air-dry.

SHOfLE

As described for malaria SHOfLE, except the biotins on the SHOfLE oligonucleotide is replaced by a fluorophore (FAM). (See Example 1.)

Microscopy

After SHOfLE hybridization, the slide is washed for 20 min in wash buffer 2, 10 min in wash buffer 3, 1 min in 96% ethanol, and air-dried. The slide is mounted using Vectashield and analyzed by fluorescence microscopy. 12 random pictures were acquired for each experimental condition and the fluorescence intensity was quantified using Image J software.

Buffers

As for malaria SHOfLE (see Example 1).

### EXAMPLE 3

### Use of SHOfLE Technology to Detect HIV Integrase

Summary

To enable SHOfLE detection of HIV integrase we immobilize the LTR (the sequence binding the HIV integrase) and allow the integrase to ligate the LTR to a double stranded DNA substrate. The ends of the double stranded DNA is designed in such a way, that the SHOfLE oligonucleotides will hybridize to them allowing detection of integration events by SHOfLE.

Cloning, Expression, and Purification of HIV Integrase

The gene encoding the HIV integrase was purchased from Geneart (Life Technologies) and cloned into the pTrcHis vector from Invitrogen using the pTrcHis TOPO® TA Expression Kit. The resulting vector enables IPTG inducible expression of His-tagged integrase in E. coli. Transformed BL21 cells were grown in LB and expression of the integrase was induced with IPTG (0.3 mM). His-tagged integrase was purified using a Ni-NTA Superflow column and eluted with elution buffer (10 mM Tris-HCl, pH7.5, 200 mM NaCl, 150 mM imidazole, 5 mM MgCl₂, 10% glycerol, 1 mM PMSF). lntegrase containing fractions were pooled and dialyzed against 200 mM KCl, 10 μM ZnCl₂, 50% glycerol. Aliquots of fractions were stored at −20° C.

Preparation of Double Stranded DNA Substrate

The following two oligonucleotides are hybridized in reaction buffer (20 mM 2-(N-morpholino) ethanesulfonic acid (MES, pH 6.2), 200 mM KCl, 10 mM MnCl₂, 10 mM DTT, and 10% glycerol). The final concentration of each oligonucleotide in the hybridization mixture was 5 μM.

(SEQ ID No: 12)
5′-tgcacgcatgtcgatgtgtcgcaatcgcatgttgtcatcgtgcatgc
atcatgacgtgctgactgaagcttccgct
(SEQ ID No: 13)
5′-tcagcacgtcatgatgcatgcacgatgacaacatgcgattgcgacac
atcgacatgcgtgcaagcggaagcttcag

Coupling of Amine LTR to Microtiter Wells

The LTR sequence that binds HIV integrase is immobilized on the substrate of Well-Coated™ Amine Binding, 8-well strip plates from G-biosciences, by coupling one strand covalently to the wells and then hybridizing the other strand to the covalently attached oligonucleotide.

Covalent Coupling of Amine Conjugated LTR Oligonucleotide to Microtiter Wells

The amine-couple oligonucleotide (5′-[AmC6T] TTTAGTCAGTGTGGAAAATCTCTAGCAGT: SEQ ID No: 6) is coupled to the Well-Coated™ Amine Binding, 8-well strip plates from G-biosciences as described for malaria SHOfLE (see Example 1).

Hybridization of Second LTR Strand to the Covalently Attached Strand

The second LTR strand (5′-[AmC6T]ACTGCTAGAGATTTTCCACACTGACTAAA; SEQ ID No: 7) hybridized to the covalently attached strand as described for malaria SHOfLE (see Example 1).

Integrase Reaction

Dilute the double stranded DNA substrate in reaction buffer (20 mM 2-(N-morpholino) ethanesulfonic acid (MES, pH 6.2), 200 mM KCl, 10 mM MnCl₂, 10 mM DTT, and 10% glycerol). The final concentration should be 0.5 μM. Add either 2% (v/v) of purified enzyme (diluted as indicated on the graph) or HIV-integrase storage buffer (200 mM KCl, 10 μM ZnCl₂, 50% glycerol). Add 50 μL to each well and incubate at 37° C. for 2 h in a humidity chamber. Wash for 1 min in wash buffer 2, 1 min in wash buffer 3, and 1 min in 96% ethanol. Let the wells air-dry.

SHOfLE

As described for malaria SHOfLE (see Example 1).

Silver Staining

As described for malaria SHOfLE (see Example 1).

Buffers

As for malaria SHOfLE (see Example 1).

### EXAMPLE 4

### Detection of Plasmodium falciparum Topoisomerase I by One-Step Assay

Summary

Plasmodium falciparum topoisomerase I (PFTopo I) is detected by immobilizing amine coupled-oligonucleotide to a surface. PFTopo I can mediate ligation of a PCR product to this oligonucleotide to form a ligation product that comprises a linear single strand of nucleic acid that can be detected.

The method is illustrated in FIGS. 17 and 18(B) and involves only one incubation step and one wash step (which itself may comprise one or more individual washes in different wash liquids). Advantages of the methodology include the fact that the substrate can be prepared beforehand, thus reducing time to readout; the background is lower (substrate more specific according to Plasmodium specificity); and sensitivity is high (see FIG. 19 which shows a detection level below 5 parasites/μl).

Methodology

Printing Slides

Day 1: Prepare slides with 2 codelink pieces on each slide: Cut Codelink slide into 5×5 mm pieces and glue them to an objective glass. Let it dry for 10-15 min and use the grease pen around the edges. Alternatively, make wells with the fatpen.

In the middle of the codelink piece/fatpen well, place a drop of 10 μL of following “coupling mixture”:

Coupling Mixture

| | |
|---|---|
| 6x print buffer | 2 μL |
| H₂O | 4 μL |
| Amine-oligo (Am-S5-Cl-37, 10 μM) | 6 μL |
| Final volume | 12 μL (10 μL pr printed area) |

The amine-oligo comprises the sequence:

(SEQ ID No: 3)
ACT ACC ATT CTG AGT CGT TCG ATC TAA AAG ACT TAG A.

The slides are places in a humidity chamber with saturated NaCl at 25° C. o/n.

Day 2: Place the slides in stands and fill with blocking buffer (only enough to cover the codelink pieces), and block for 30 min at 50° C. Wash twice in ion-exchanged water (2×1 min). Wash for 30 min at 50° C. in wash buffer 1 (4×SSC+0.1% SDS). Wash twice in ion-exchanged H₂O for 1 min (place on shaker). Let the slides air dry.

FROM NOW, EVERYTHING SHOULD BE DONE IN DIMMED
LIGHT.

Hybridization of PCR Product to Primers

| | |
|---|---|
| TEN3 | 9 μL |
| 2.5 or 5 ng PCR product (adjusted to 2.5 or 5 ng/μL) | 1 μL |

The PCR product is produced using the vector pYES2.1 TOPO (Invitrogen) as a template.

Primers:

P1:
(SEQ ID No: 25)
ATT TTT CTA AGT CTT TTA GAT CGA ACG ACT CAG AAT
GAT GCA TGT ATA CTA AAC TCA CAA ATT AGA GC
P2:
(SEQ ID No: 26)
TTT TTT TTT TTT TTT TTT TTT TTT TGC TTT CTC ATA
GCT CAC GCT G

The generated PCR fragment is around 1500 bp and hybridizes to the following oligo:

(SEQ ID No: 3)
ACT ACC ATT CTG AGT CGT TCG ATC TAA AAG ACT TAG A

Place 10 μL per well.

Incubate 1 hour at 37 or 50° C. in humidity chamber.

Wash the slides for 1 min in wash buffer 2 (place on shaker). Wash for 1 min in wash buffer 3 (place on shaker).

Dehydrate the slides for 1 min in 99% EtOH (place on shaker). Let the slides air dry.

Cleavage of Substrate:

TEN3 9 μL

pfTop1/blood lysate 1 μL

Incubate the slides for 60 min at 37° C. in a humidity chamber.

The nucleic acid substrate that is recognised by topoisomerase in this Example is made up of the amine-linked oligonucleotide and the part of the PCR product that it is hybridised to. The amine-linked oligonucleotide is cleaved by topoisomerase and then ligated to the part of the PCR product that it is hybridised to.

Wash:

| | |
|---|---|
| TE-500 mM NaCl | 1 min |
| Wash 2 | 1 min |
| Wash 3 | 1 min |
| Water | 1 min |
| Urea (8M) | 30 min |
| Urea (8M) | 30 min |
| Wash 2 | 1 min |
| Wash 3 | 1 min |
| 96% EtOH | 1 min |

Let airdry.

Mount each slide with 2 μL Vectashield without DAPI. Put on coverglass. Add a small drop of immersion oil on the coverglass and analyse under 63× objective.

Buffers

10*TE:

100 mM Tris pH 7.5 (for 100 mL, use 10 mL of 1M)

10 mM EDTA (for 5 mL of 200 mM)

TEN3:

1 mL of 3M NaCl

1 mL of 10*TE

7 mL Water

Preparation of Blood-Lysate

320 μL lysis buffer (10 mM Tris HCl, pH 7.5; EDTA; protease inhibitors (complete, Roche))+80 μL blood.

Mix with 100 μL glass-beads, bead beat for 3 min.

Leave on ice for 15 min.

Centrifuge at 1000 g for 5 min.

Use instead of pfTopo in One-Step-assay protocol.

Sequences

| Mycobacterium tuberculosis topoisomerase I gene sequence: | |
|---|---|
| SEQ ID NO: 18 | |
| TTGGCTGACCCGAAAACGAAGGGCCGTGGCAGCGGCGGCAATGGCAGCGGCCGGCGACTGGTCAT | |
| CGTCGAGTCGCCCACCAAGGCGCGCAAGCTGGCCTCCTACCTGGGCTCTGGCTACATCGTCGAGT | |
| CCTCCCGGGGGCACATCCGTGACTTGCCGCGGGCCGCGTCGGATGTACCCGCAAAGTACAAGTCG | |
| CAGCCGTGGGCGCGGCTCGGGGTCAACGTCGACGCCGACTTCGAACCGCTCTACATCATCAGCCC | |
| GGAGAAACGGAGCACCGTCAGCGAGCTCAGGGGCCTGCTCAAAGACGTGGACGAGCTGTATCTGG | |
| CCACGGATGGGGACCGTGAGGGCGAAGCTATTGCCTGGCATCTGCTGGAAACCCTCAAACCGCGC | |
| ATACCGGTAAAGCGGATGGTCTTCCACGAGATCACCGAACCGGCGATCCGCGCCGCCGCCGAGCA | |
| CCCCCGCGACCTAGACATCGACCTGGTCGACGCGCAGGAGACCCGGCGCATCCTGGACCGGCTGT | |
| ACGGCTACGAAGTCAGCCCAGTGCTGTGGAAGAAGGTCGCCCCCAAGTTGTCGGCGGGCCGGGTG | |
| CAGTCGGTGGCCACCCGCATCATCGTGGCGCGCGAACGCGACCGCATGGCGTTCCGCAGCGCGGC | |
| CTACTGGGACATCCTTGCCAAGCTGGATGCCAGCGTGTCCGACCCGGACGCCGCGCCGCCCACCT | |
| TCAGCGCCCGGCTGACGGCCGTGGCTGGCCGGCGGGTGGCCACTGGCCGCGATTTCGACTCGCTG | |
| GGCACGCTGCGCAAAGGCGACGAAGTCATTGTGCTCGACGAGGGGAGCGCGACCGCGTTGGCCGC | |
| GGGCCTGGATGGCACGCAGCTGACCGTGGCCTCGGCCGAGGAGAAGCCCTACGCCCGGCGCCCGT | |
| ACCCGCCGTTCATGACCTCCACGCTGCAGCAAGAGGCCAGCCGCAAGCTGCGGTTCTCCGCCGAG | |
| CGGACGATGAGCATCGCCCAGCGGCTGTACGAAAACGGCTACATCACCTATATGCGTACCGACTC | |
| CACCACGCTGTCGGAGTCGGCGATCAACGCCGCACGTACCCAGGCGCGCCAGCTCTACGGCGACG | |
| AGTACGTCGCGCCGGCGCCGCGCCAATACACCCGCAAGGTGAAGAACGCCCAGGAAGCGCACGAG | |
| GCTATCCGGCCCGCCGGTGAAACGTTTGCCACCCCGGACGCGGTGCGTCGCGAACTCGACGGTCC | |
| CAACATTGATGATTTCCGGCTCTATGAGCTGATTTGGCAACGCACCGTAGCCTCGCAGATGGCCG | |
| ATGCGCGGGGCATGACGCTGAGCCTGCGGATCACTGGCATGTCGGGGCACCAGGAGGTGGTGTTC | |
| TCCGCGACCGGACGCACCTTGACGTTCCCGGGCTTCCTCAAGGCCTACGTGGAGACCGTGGACGA | |
| GCTGGTCGGCGGCGAGGCTGACGATGCCGAGCGGCGACTGCCCCATCTGACCCCGGGTCAACGGT | |
| TGGACATCGTCGAGTTGACCCCAGACGGCCATGCCACCAACCCGCCGGCCCGCTACACCGAGGCG | |
| TCGCTGGTCAAAGCGCTCGAGGAGCTGGGCATCGGCCGCCCGTCGACCTACTCGTCGATCATCAA | |
| GACCATCCAGGATCGCGGCTACGTGCACAAGAAGGGCAGTGCACTGGTGCCGTCATGGGTGGCGT | |
| TCGCGGTAACCGGTCTGCTCGAGCAGCATTTCGGTCGGCTCGTCGACTACGACTTCACCGCGGCG | |
| ATGGAAGACGAGCTCGACGAGATCGCCGCCGGCAACGAGCGCCGCACCAACTGGCTCAACAACTT | |
| CTACTTTGGTGGCGATCACGGTGTGCCCGATTCGGTAGCCCGATCGGGTGGCCTCAAGAAGCTTG | |
| TCGGGATCAATCTCGAGGGCATCGACGCACGAGAAGTAAACTCTATCAAGCTTTTTGACGACACC | |
| CACGGACGCCCCATATATGTTCGGGTGGGCAAGAACGGTCCCTACCTGGAACGTTTGGTGGCCGG | |
| CGACACCGGTGAGCCCACGCCGCAGCGGGCCAACCTCAGCGACTCGATTACCCCGGACGAGCTGA | |
| CTCTACAGGTGGCCGAAGAGCTCTTTGCCACACCGCAACAGGGACGGACTTTGGGCTTGGACCCA | |
| GAAACCGGCCACGAGATCGTGGCCAGGGAAGGCCGGTTTGGGCCGTATGTGACCGAGATCCTGCC | |
| GGAGCCTGCGGCTGATGCGGCCGCGGCCGCTCAGGGAGTCAAGAAACGCCAGAAGGCCGCCGGGC | |
| CCAAACCGCGCACCGGTTCGTTGCTGCGGAGCATGGACCTACAGACGGTCACCCTCGAAGACGCG | |
| CTGAGGCTGCTGTCACTGCCGCGCGTGGTCGGAGTGGACCCCGCCTCGGGTGAGGAGATCACCGC | |
| GCAGAACGGGCGCTACGGACCGTATCTAAAGCGCGGCAACGATTCTCGATCACTGGTCACCGAAG | |
| ACCAGATATTCACCATCACGCTCGACGAAGCCCTGAAGATCTACGCAGAGCCGAAACGTCGTGGC | |
| CGGCAAAGCGCTTCGGCTCCGCCGCTGCGCGAGCTGGGAACAGATCCGGCGTCGGGCAAGCCAAT | |
| GGTCATCAAGGACGGCCGATTCGGGCCGTACGTCACCGACGGTGAGACCAATGCCAGCCTGCGTA | |
| AGGGCGACGACGTGGCTTCCATAACCGACGAGCGCGCCGCCGAGCTGTTGGCCGATCGCCGAGCC | |
| CGGGGTCCGGCAAAACGGCCAGCCAGGAAAGCTGCCCGGAAGGTGCCGGCGAAGAAGGCAGCCAA | |
| GCGCGACTAG | |
| Mycobacterium tuberculosis topoisomerase I proteinsequence: | |
| SEQ ID NO: 19 | |
| MADPKTKGRGSGGNGSGRRLVIVESPTKARKLASYLGSGYIVESSRGHIRDLPRAASDVPAKYKS | |
| QPWARLGVNVDADFEPLYIISPEKRSTVSELRGLLKDVDELYLATDGDREGEAIAWHLLETLKPR | |
| IPVKRMVFHEITEPAIRAAAEHPRDLDIDLVDAQETRRILDRLYGYEVSPVLWKKVAPKLSAGRV | |
| QSVATRIIVARERDRMAFRSAAYWDILAKLDASVSDPDAAPPTFSARLTAVAGRRVATGRDFDSL | |
| GTLRKGDEVIVLDEGSATALAAGLDGTQLTVASAEEKPYARRPYPPFMTSTLQQEASRKLRFSAE | |
| RTMSIAQRLYENGYITYMRTDSTTLSESAINAARTQARQLYGDEYVAPAPRQYTRKVKNAQEAHE | |
| AIRPAGETFATPDAVRRELDGPNIDDFRLYELIWQRTVASQMADARGMTLSLRITGMSGHQEVVF | |
| SATGRTLTFPGFLKAYVETVDELVGGEADDAERRLPHLTPGQRLDIVELTPDGHATNPPARYTEA | |
| SLVKALEELGIGRPSTYSSIIKTIQDRGYVHKKGSALVPSWVAFAVTGLLEQHFGRLVDYDFTAA | |
| MEDELDEIAAGNERRTNWLNNFYFGGDHGVPDSVARSGGLKKLVGINLEGIDAREVNSIKLFDDT | |
| HGRPIYVRVGKNGPYLERLVAGDTGEPTPQRANLSDSITPDELTLQVAEELFATPQQGRTLGLDP | |
| ETGHEIVAREGRFGPYVTEILPEPAADAAAAAQGVKKRQKAAGPKPRTGSLLRSMDLQTVTLEDA | |
| LRLLSLPRVVGVDPASGEEITAQNGRYGPYLKRGNDSRSLVTEDQIFTITLDEALKIYAEPKRRG | |
| RQSASAPPLRELGTDPASGKPMVIKDGRFGPYVTDGETNASLRKGDDVASITDERAAELLADRRA | |
| RGPAKRPARKAARKVPAKKAAKRD | |
| P.F. TopI | |
| For information, cf: | |
| http://www.ncbi.nlm.nih.gov/gene/812833 | |
| Plasmodium falciparum Gene sequence (ACCESSION NC_004326): | |
| http://www.ncbi.nlm.nih.qov/nuccore/NC_004326?report=genbank&from= | |
| 445981&to=448500&strand=true | |
| SEQ ID NO: 20 | |
| 1 | atgcaatcaa tggaaataaa tgataataac agtatcaaga atgaaagtac atctgatgat |
| 61 | gatatattaa ttaataaaat taaacaaaac ttgggtaata ataaatcatg taattctaga |
| 121 | tcttccaaaa aggaatctat aaaaaagcaa aagagcaatt ctgaacttgg tataaaaaag |
| 181 | aacacaaaga aatcattagg tataaaaaaa gaggaagaaa aaaaaaaaca aataagcaaa |
| 241 | agaaaaagta atgaactaaa agaaaaaaat aatttgaaag agggaaaaaa gaaatatgtg |
| 301 | gaaaaaaaat ctagaacagt aaaagatgaa accaagttaa cgaatgttat aaaaaaagaa |
| 361 | actcaaaata ataagaaacc taaaaaatta cttaaaaaat cagaagaaaa ttttgaacca |
| 421 | ataaatagat ggtgggaaaa aatagatgat caaacagata tacaatggaa ttatttagaa |
| 491 | catcgaggat taatattttc ccctccatac gttcaacatc atgtaccaat tttttataaa |
| 541 | agtataaaaa ttgaattaaa tgcaaaatca gaagaattag ctacctattg gtgtagtgca |
| 601 | attggtagtg attattgtac aaaagaaaag tttatattaa atttttttaa aacatttata |
| 661 | aatagtttag aaaatgataa tattataaaa caagagaatg aaacgaaatt aaaaaaagga |
| 721 | gatatatcta attttaagtt tattgatttt atgccaatca aagatcattt attaaaatta |
| 781 | agagaagaaa agttaaataa aacaaaagaa gaaaaagaag aggaaaaaaa aatgagaatg |
| 841 | gaaaaagaat taccatatac atatgcgtta gttgattgga ttcgtgaaaa gatatcaagt |
| 901 | aataaagcag aaccacctgg gttatttaga ggaagaggag aacatccaaa acaaggttta |
| 961 | ttaaaaaaaa gaatttttcc agaagatgtt gtaattaata ttagtaaaga tgcacctgta |
| 1021 | ccacgattat atgataatat gtgtggacat aattggggtg atatatatca tgataataaa |
| 1081 | gtaacatggt tagcttatta taaagatagt ataaatgatc aaataaaata tactttttta |
| 1141 | tctgctcaat caaaatttaa aggatataaa gatcttatga aatatgaaaa tgctcgaaaa |
| 1201 | ttaaaatcat gtgttcataa aattagggaa gattataaaa ataaaatgaa aaataaaaat |
| 1261 | attattgata aacaattagg aacagctgtt tatttaatag attttctagc attaagagta |
| 1321 | ggaggagaaa aagatatcga tgaagaagca gatactgtag gttgttgtag tttaagagta |
| 1381 | gaacatatta gttttgcaca cgatatacct tttaaaagtg tagattcaaa agaacaaaaa |
| 1441 | acaaatgatg aaaaagtaaa taaaatacca ttaccaacaa atttagaaag tatttcatca |
| 1501 | gaagattgtt atataacttt agatttttta ggaaaagata gtatacgata ttttaataca |
| 1561 | gtcaaaatag ataaacaagc atatattaat ataataatat tttgtaaaaa taaaaataga |
| 1621 | gatgaaggag tttttgatca aataacttgt tcaaaattaa atgaatatct aaaagaaatt |
| 1681 | atgcctactt tatcagctaa agtgtttcgt acatataatg cttcaattac attagatcaa |
| 1741 | caattaaaaa gaataaaaga agtttatgga aaaacaacat attcattata ttctggtgaa |
| 1801 | acagaattac acaaatcgaa aaaaagaaaa tctagccatt taacttcaga tacaaatata |
| 1861 | ttaagtgatg caagtgattc tactattaat gatgtaaata acgagtatga tgaaaatgga |
| 1921 | ataaataaaa aactatcata tgctactact gtaggaaaag aaaatgatgt cgatgataaa |
| 1981 | aactcaccaa tagaagttga cgtttcaaat ataaatgaac ttattaattt ttacaataat |
| 2041 | gcaaatagag aagtagccat attatgtaac catcaaagaa gtattccaaa acaacatgat |
| 2101 | acaactatgt caaaaataaa aaaacaaatt gaattatata atgaagatat aaaagaatat |
| 2161 | aaaaaatatt tgcaacattt aaaaaaaaat agtgataaaa aatttatctt tgtttcgaaa |
| 2221 | gtttctactt tagatggaac tttaagacca aataaagtca aagaaaatat gaaagaagaa |
| 2281 | tcttgtaaaa aaaaactaat tactcttata aaaaaagttg aattattaaa taaccaaatg |
| 2341 | aaagtaagag atgataataa aactattgct ttaggtacat ctaaaattaa ttatatggat |
| 2401 | ccaagaataa ctgttgcttt ttgtaaaaaa tttgaaatac ccatagaaaa agtatttaat |
| 2461 | agaagtttaa gacttaaatt tccttgggcc atgtttgcta caaaaaattt tacattttaa |
| Plasmodium falciparum Protein sequence (ACCESSION XP_001351663): | |
| http://www.ncbi.nlm.nih.gov/protein/XP_001351663.1 | |
| SEQ ID NO: 21 | |
| 1 | mqsmeindnn siknestsdd dilinkikqn lgnnkscnsr sskkesikkq ksnselgikk |
| 61 | ntkkslgikk eeekkkqisk rksnelkekn nlkegkkkyv ekksrtvkde tkltnvikke |
| 121 | tqnnkkpkkl lkkseenfep inrwwekidd qtdiqwnyle hrglifsppy vqhhvpifyk |
| 181 | sikielnaks eelatywcsa igsdyctkek filnffktfi nslendniik qenetklkkg |
| 241 | disnfkfidf mpikdhllkl reeklnktke ekeeekkmrm ekelpytyal vdwirekiss |
| 301 | nkaeppglfr grgehpkqgl lkkrifpedv viniskdapv prlydnmcgh nwgdiyhdnk |
| 361 | vtwlayykds indqikytfl saqskfkgyk dlmkyenark lkscvhkire dyknkmknkn |
| 421 | iidkqlgtav ylidflalrv ggekdideea dtvgccslry ehisfahdip fksvdskeqk |
| 481 | tndekvnkip lptnlesiss edcyitldfl gkdsiryfnt vkidkqayin iiifcknknr |
| 541 | degvfdqitc sklneylkei mptlsakvfr tynasitldq qlkrikevyg kttyslysge |
| 601 | telhkskkrk sshltsdtni lsdasdstin dvnneydeng inkklsyatt vgkendvddk |
| 661 | nspievdvsn inelinfynn anrevailcn hqrsipkqhd ttmskikkqi elynedikey |
| 721 | kkylqhlkkn sdkkfifvsk vstldgtlrp nkvkenmkee sckkklitli kkvellnnqm |
| 781 | kvrddnktia lgtskinymd pritvafckk feipiekvfn rslrlkfpwa mfatknftf |
| Streptococcus agalactiae DNA topoisomerase I amino acid sequence | |
| (ACCESSION WP_000246605): | |
| SEQ ID NO: 27 | |
| 1 | matttktstk ktskkksata kknlvivesp akaktiekyl grnykvvasv ghirdlkkss |
| 61 | msidfennye pqyinirgkg plindlkkea kkakkvylas dpdregeais whlahildld |
| 121 | kedrnrvvfn eitkdavkna fveprqinmd lvdaqqarrv ldrivgysis pilwkkvkkg |
| 181 | lsagrvqsva lkliidrene ikafqpeeyw tidgsfkkgt rkfnatfygl dgkkfklsnn |
| 241 | edvktvlkri ktdeflvekv ekkerprnap lpyttsslqq daankinfrt rktmmiaqql |
| 301 | yeglslgtag hqglitymrt dstrisplaq neaaefitnr fganyskhgn kvknasgaqd |
| 361 | aheairpssv nhtpesiaky ldkdqlklyt liwnrfiasq mtaavfdtmk vnltqngvtf |
| 421 | iangsqvkfd gymavyndtd knkmlpdmee gesvkkvntn peqhftqppa rfseaslikt |
| 481 | leengvgrps tyaptletiq kryyvklaak rfeptelgei vnsliveffp divdvtftae |
| 541 | megkldevei gkeqwqkiid efykpfekel akaetemeki qikdepagfd celcgspmvi |
| 601 | klgrygkfya csnfpechnt kaitkeigvi cpicqkgqvi erktkrnrif ygcdrypece |
| 661 | ftswdkpigr tcpksndflv ekkvrgggkq vvcsnekcdy qeekik |

1. A method of identifying a microorganism that expresses a nucleic acid-modifying enzyme, in a sample, the method comprising:
(a) contacting a nucleic acid substrate targeted by the nucleic acid-modifying enzyme with the sample;
(b) adding a further nucleic acid molecule to the sample which nucleic acid molecule is ligated to the nucleic acid substrate in the presence of the nucleic acid-modifying enzyme to form a ligation product which comprises a linear single strand of nucleic acid that is capable of being detected; and
(c) detecting the presence of the ligation product.

2. A method according to claim 1, wherein step (a) comprises
(i) contacting a nucleic acid substrate targeted by the nucleic acid-modifying enzyme with the sample, wherein the nucleic acid substrate is immobilised on a surface; and
(ii) washing the nucleic acid substrate immobilised on the surface so as to remove substantially all non-specifically bound material from the substrate.

3. A method according to claim 1 or 2, wherein the nucleic acid-modifying enzyme is a DNA modifying enzyme.

4. A method according to any of claims 1-3, wherein the nucleic acid-modifying enzyme is a ligase such as a topoisomerase (e.g. a type I topoisomerase), an integrase, a recombinase or a transposase.

5. A method according to any of claims 1-4, wherein the microorganism is a pathogenic microorganism or a parasitic microorganism.

6. A method according to any of claims 1-5, wherein the microorganism is selected from the group consisting of a virus, a bacteria, a protozoa, a fungus, a mould, an amoeba or a parasitic worm.

7. A method according to any of claims 1-6, wherein the microorganism belongs to the Mycobacteriaceae family, such as one selected from the Mycobacterium genus, optionally wherein the microorganism is Mycobacterium tuberculosis, Mycobacterium bovis, or Mycobacterium smegmatis.

8. A method according to any of claims 1-5, wherein the microorganism belongs to the Plasmodiidae family, such as one selected from the Plasmodium genus, optionally wherein the microorganism is Plasmodium falciparum.

9. A method according to any of claims 1-5, wherein the microorganism is a Streptococcus such as Group B Streptococcus.

10. A method according to any of claims 1-5, wherein the microorganism is a virus such as a retrovirus, optionally wherein the retrovirus is any of Human Immunodeficiency Virus (HIV), simian immunodeficiency virus (SIV), murine leukaemia virus (MLV) or felineimmunodeficiency virus (FIV).

11. A method according to any of claims 1-10, wherein the sample is food or water.

12. A method, according to any of claims 1-10, wherein the sample is from a mammal.

13. A method according to claim 12, wherein the sample is from a human, a cow, a ferret, a badger, a koala, a chicken, a turkey, a duck, a rodent, an elephant, a bird, a pig, a deer, a coyote, a camel, a puma, a fish, a dog, a sheep, a goat, a cat, or a non-human primate.

14. A method according to claim 12 or 13, wherein the sample is blood plasma, blood serum, whole blood, sputum, saliva, urine, a cell smear, faeces, cerebrospinal fluid or a biopsy.

15. A method according to any of claims 1-14 wherein step (a) comprises incubating the nucleic acid substrate and sample at a temperature of between 30-40° C., preferably 37° C.

16. A method according to any of claims 1-15, wherein the nucleic acid substrate is selectively targeted by the nucleic acid-modifying enzyme from the microorganism.

17. A method according to any of claims 1-16, wherein the nucleic acid substrate is single stranded or double stranded.

18. A method according to any of claims 1-17, wherein the microorganism is selected from the Plasmodium genus, and the nucleic acid substrate is double stranded DNA wherein at least one strand comprises a sequence selected from the group consisting of ACTACCATTCTGAGTCGTTCGAAGTTCCTATACTTT (SEQ ID No: 1) and TCTAGAAAGTATAGGAACTTCGAACGACTCAGAATG (SEQ ID No: 2); a sequence sharing at least 90% sequence identity thereto; or a sequence which is a part of at least 5 consecutive nucleotides of any of said sequences.

19. A method according to any of claims 1-17, wherein the microorganism is selected from the Plasmodium genus, and the nucleic acid substrate is double stranded DNA wherein at least one strand comprises a sequence selected from the group consisting of ACTACCATTCTGAGTCGTTCGATCTAAAAGACTTAGA (SEQ ID No: 3) and ATTTTTCTAAGTCTTTTAGATCGAACGACTCAGAATG (SEQ ID No: 4); a sequence sharing at least 90% sequence identity thereto; or a sequence which is a part of at least 5 consecutive nucleotides of any of said sequences.

20. A method according to any of claims 1-17, wherein the microorganism is selected from the Mycobacterium genus, and the nucleic acid substrate is single stranded DNA that comprises the sequence CAGTGAGCGAGCTTCCGCTTGACATCCCAATAGTTTCTCTTC (SEQ ID No: 5); a sequence sharing at least 90% sequence identity thereto; or a sequence which is a part of at least 5 consecutive nucleotides of any of said sequence.

21. A method according to any of claims 1-17, wherein the microorganism is a virus, and the nucleic acid substrate is a long terminal repeat (LTR) sequence that is selectively targeted by an integrase of the virus.

22. A method according to claim 21, wherein the nucleic acid substrate is double stranded DNA wherein at least one strand comprises a sequence selected from the group consisting of TTTAGTCAGTGTGGAAAATCTCTAGCAGT (SEQ ID No: 6) and ACTGCTAGAGATTTTCCACACTGACTAAA (SEQ ID No: 7); a sequence sharing at least 90% sequence identity thereto; or a sequence which is a part of at least 5 consecutive nucleotides of any of said sequences; or wherein the nucleic acid substrate is double stranded DNA wherein at least one strand comprises a sequence selected from the group consisting of TTGACTACCCGTCAGCGGGGGTCTTTCATT (SEQ ID No: 22) and AATGAAAGACCCCCGCTGACGGGTAGTCAA (SEQ ID No: 23); a sequence sharing at least 90% sequence identity thereto; or a sequence which is part of at least 5 consecutive nucleotides of any of said sequences.

23. A method according to claim 22, wherein the nucleic acid substrate is double stranded DNA wherein the first strand comprises the sequence TTTAGTCAGTGTGGAAAATCTCTAGCAGT (SEQ ID No: 6) and the second strand comprises the sequence ACTGCTAGAGATTTTCCACACTGACTAAA (SEQ ID No: 7), or wherein the nucleic acid substrate is double stranded DNA wherein the first strand comprises the sequence TTGACTACCCGTCAGCGGGGGTCTTTCATT (SEQ ID No: 22) and the second strand comprises the sequence AATGAAAGACCCCCGCTGACGGGTAGTCAA (SEQ ID No: 23).

24. A method according to any of claims 1-23, wherein the surface is a solid support.

25. A method according to claim 24, wherein the solid support is glass, paper, cardboard, sepharose, agarose, plastic, metal, silicon, ceramics and latex.

26. A method according to claim 25, wherein the solid support is coated with maleic anhydride.

27. A method according to any of claims 1-26, wherein ligation of the further nucleic acid molecule to the nucleic acid substrate does not generate a circular ligation product.

28. A method according to any of claims 1-27, wherein the ligation product is formed directly following ligation of the further nucleic acid molecule to the nucleic acid substrate.

29. A method according to any of claims 1-27, wherein the ligation product is formed indirectly via the formation of one or more intermediate products following ligation of the further nucleic acid molecule to the nucleic acid substrate.

30. A method according to any of claims 1-29, wherein the further nucleic acid molecule is double stranded DNA or single stranded DNA.

31. A method according to claim 30, wherein the further nucleic acid molecule is double stranded DNA, the nucleic acid substrate is a long terminal repeat (LTR) sequence that is selectively targeted by an integrase of the virus, and the nucleic acid-modifying enzyme is an integrase.

32. A method according to any of claims 1-31, wherein the ligation product is one that comprises a linear single strand of nucleic acid that is capable of being detected by hybridising to one or more detectable oligonucleotides, or that is capable of being detected by comprising one or more detectable nucleotides.

33. A method according to claim 32, wherein the one or more detectable oligonucleotides are capable of forming, by self-assembly, with other such one or more detectable oligonucleotides, substantially contiguous single strands of a double-stranded nucleic acid filament.

34. A method according to claim 32 or 33, wherein the one or more detectable oligonucleotides or nucleotides are detectably labelled with one or more fluorescent dyes, radioactive nucleotides or biotinylated nucleotides, or wherein the one or more detectable oligonucleotides or nucleotides are detectably labelled by being coupled to an enzyme, which enzyme is capable of converting a substrate into a detectable product.

35. A method according to claim 34, wherein the one or more detectable oligonucleotides comprise biotinylated nucleotides which are detected by using streptavidin coupled gold nanoparticles.

36. A method according to any of claims 32-35, wherein the one or more detectable oligonucleotides comprise a sequence selected from the group consisting of CAG TGA GCG TCT GGC TGA AGC TTC CGC T (SEQ ID No: 16) and CCA GAC GCT CAC TGA GCG GAA GCT TCA G (SEQ ID No: 17).

37. A method according to any of claims 30-36, wherein the further nucleic acid molecule is double stranded DNA wherein the first strand comprises the sequence TGCACGCATGTCGATGTGTCGCAATCGCATGTTGTCATCGTGCATGCATCATGACG TGCTGACTGAAGCTTCCGCT (SEQ ID No: 12) and the second strand comprises the sequence TCAGCACGTCATGATGCATGCACGATGACAACATGCGATTGCGACACATCGACATG CGTGCAAGCGGAAGCTTCAG (SEQ ID No: 13).

38. A method according to any of claims 30-36, wherein the further nucleic acid molecule is single stranded DNA, the nucleic acid substrate is single or double stranded DNA that is selectively targeted by a type I topoisomerase, and the nucleic acid-modifying enzyme is a type I topoisomerase.

39. A method according to claim 38, wherein the further nucleic acid molecule comprises the sequence CAGTGAGCGTCTGGCTGAAGCTTCCGCT (SEQ ID No: 14) or TTCTAGACCAGACGCTCACTGAGCGGAAGCTTCAG (SEQ ID No: 15).

40. A method according to any of claims 1-39, wherein step (c) is followed by a wash step to remove substantially all further nucleic acid molecule that is non-ligated to the substrate.

41. A method of identifying a microorganism that expresses a nucleic acid-modifying enzyme, in a sample, the method comprising:
(a) contacting a nucleic acid substrate targeted by the nucleic acid-modifying enzyme with the sample, wherein the nucleic acid substrate is capable of being processed in the presence of the nucleic acid modifying enzyme to form a ligation product which comprises a linear single strand of nucleic acid that is capable of being detected; and
(b) detecting the presence of the ligation product.

42. A method of identifying a microorganism that expresses a nucleic acid-modifying enzyme, in a sample, the method comprising:
(a) contacting a nucleic acid substrate targeted by the nucleic acid-modifying enzyme with the sample, wherein the nucleic acid substrate comprises a first strand of nucleic acid that is immobilised on a surface and a second strand of nucleic acid that is hybridised to the first strand of nucleic acid but which is not immobilised to the surface, and wherein, in the presence of the nucleic acid modifying enzyme, the first strand of nucleic acid is capable of being ligated to the second strand of nucleic acid to form a ligation product which comprises a linear single strand of nucleic acid that is capable of being detected,
(b) washing the nucleic acid substrate immobilised on the surface so as to remove substantially all non-specifically bound material from the substrate, under denaturing conditions; and
(c) detecting the presence of the ligation product.

43. A method according to claim 42, wherein the surface is a bead or a slide.

44. A method according to claim 43, wherein the bead is a streptavidin coated bead and the first strand of the nucleic acid substrate is a biotinylated strand of nucleic acid.

45. A method according to claim 44, wherein the slide is a codelink slide and the first strand of the nucleic acid substrate is an amine-linked strand of nucleic acid.

46. A method according to any of claims 1-45, wherein the presence of the ligation product is detected by any one or more techniques selected from the group consisting of a linear amplification technique, southern blotting, polymerase chain reaction (PCR), reverse-transcription-PCR (RT-PCT), quantitative PCR (qPCR), restriction fragment length dimorphism-PCR (RFLD-PCR), primer extension, DNA array technology, and isothermal amplification.

47. A method according to any of claims 1-45, wherein the presence of the ligation product is detected by an amplification technique that does not involve use of a polymerase.

48. A method according to claim 47, wherein the amplification technique involves the use of one or more detectable oligonucleotides as defined in any of claims 32-36.

49. A method according to any of claims 1-48, wherein the presence of the ligation product is detected in a microfluidic system.

50. A method according to claim 49, comprising:
(i) loading the washed nucleic acid substrate from step (b) and the further nucleic acid molecule from step (c) into a sample chamber comprising a flow through channel, wherein droplets comprising the nucleic acid substrate are generated;
(ii) transferring the droplets from the sample chamber to a droplet retaining means through the flow through channel;
(iii) capturing one or more single droplets in individual cavities of the droplet retaining means, wherein each single droplet is spatially isolated from other droplets; and
(iv) detecting, in one or more captured droplets, the ligation product.

51. A method according to claim 50, wherein the sample chamber comprises one or more inlet channels and/or one of more outlet channels for the generated drops.

52. A method according to claim 50 or 51, wherein the one or more flow through channels, inlet channels and/or outlet channels have a diameter of 10-50 micrometers, such as approximately 25 micrometers.

53. A method of diagnosing an infectious disease in a subject, the method comprising identifying a microorganism in a sample from the subject according to the method of any of claims 1-52, wherein the presence of the microorganism in the sample is indicative of the infectious disease.

54. A method according to claim 53, wherein the disease is tuberculosis; or malaria; or Group B Streptococcus disease; or a retroviral infection such as HIV infection or FIV infection or MLV infection or SIV infection; or cancer.

55. A method according to claim 53 or 54, wherein the subject is a mammal, a human, a cow, a ferret, a badger, a koala, a chicken, a turkey, a duck, a rodent, an elephant, a bird, a pig, a deer, a coyote, a camel, a puma, a fish, a dog, a sheep, a goat, a cat, or a non-human primate.

56. A method of combating an infectious disease in a subject, the method comprising diagnosing the disease according to the methods of any of claims 53-55, and treating the disease.

57. An anti-infectious disease agent for use in combating an infectious disease in a subject who has been diagnosed with the disease according to the methods of any of claims 53-55.

58. A method of assessing whether an agent has an effect on an infectious disease caused by a microorganism in a subject, the method comprising administering the agent to the subject and determining the effect of the agent on the amount of ligation product produced in a sample from the subject when the sample is contacted with a nucleic acid substrate targeted by a nucleic acid-modifying enzyme of the microorganism and a further nucleic acid molecule that is ligated to the nucleic acid substrate in the presence of the nucleic acid-modifying enzyme, according to the method of any of claims 1-52.

59. A method of assessing whether an agent has an effect on a microorganism in a sample, the method comprising:
(a) contacting the sample with the agent; and
(b) assessing the effect of the agent on the amount of ligation product produced when the sample is contacted with a nucleic acid substrate targeted by a nucleic acid-modifying enzyme of the microorganism and a further nucleic acid molecule that is ligated to the nucleic acid substrate in the presence of the nucleic acid-modifying enzyme, according to the method of any of claims 1-53.

60. A method of assessing the presence or activity of a nucleic acid-modifying enzyme in a sample, the method comprising:
(a) contacting a nucleic acid substrate targeted by the nucleic acid-modifying enzyme with the sample,
optionally wherein the nucleic acid substrate is immobilised on a surface, and the nucleic acid substrate immobilised on the surface is washed so as to remove substantially all non-specifically bound material from the substrate;
(b) adding a further nucleic acid molecule to the sample which nucleic acid molecule is ligated to the nucleic acid substrate in the presence of the nucleic acid-modifying enzyme to form a ligation product that comprises a linear single strand of nucleic acid that is capable of being detected; and
(c) detecting the presence or activity of the ligation product.

61. A device for carrying out a method according to any of claims 1-52 and 60.

62. A device according to claim 61, wherein the device comprises a means for washing the nucleic acid substrate immobilised on the surface according to step (a)(i) of claim 1.

63. A device according to claim 61 or 62, wherein the device comprises a separate reaction chamber and detection chamber.

64. A device according to any of claims 61-63, wherein the device comprises a control chamber.

65. A device according to any of claims 61-64, wherein the device comprises a waste outlet.

66. A device according to any of claims 61-65, wherein the device comprises a means that allows the ligation product to be detected by an amplification technique according to claim 47 or 48.

67. A device according to any of claims 61-66, further comprising one or more analytical means to detect the ligation product.

68. A kit of parts comprising a nucleic acid substrate targeted by a nucleic acid-modifying enzyme and a means for detecting a ligation product formed by ligation of the nucleic acid substrate to a further nucleic acid molecule, which ligation product comprises a linear single strand of nucleic acid that is capable of being detected.

69. A kit of parts according to claim 68, wherein the nucleic acid substrate is immobilised on a surface, optionally as a surface as defined in any of claims 24-26.

70. A kit of parts according to claim 68 or 69, wherein the means for detecting the ligation product comprises one or more detectable oligonucleotides as defined in any of claims 33-36.

71. A kit of parts according to any of claims 68-70, wherein the nucleic acid substrate is as defined in any of claims 16-23.

72. A kit of parts according to any of claims 68-71, wherein the kit further comprises a further nucleic acid molecule that is capable of being ligated to the nucleic acid substrate in the presence of the nucleic acid-modifying enzyme to form a ligation product which comprises a linear single strand of nucleic acid that is capable of being detected.

73. A kit of parts according to claim 72, wherein the further nucleic acid molecule is as defined in any of claims 30, 31 and 37-39.

74. A kit of parts according to any of claims 68-73, which kit does not comprise a polymerase.

75. A kit of parts according to any of claims 68-74, wherein the kit comprises a plurality of, nucleic acid substrates each targeted by a nucleic acid-modifying enzyme of a different microorganism, cell or cell type.

76. A kit of parts according to any of claims 68-75, wherein the kit comprises an agent for depletion of divalent cations.


SEQUENCE LISTING (28 sequences)

SEQ ID NO: 1 - DNA, 36 bp, Artificial Sequence
  source 1..36: /mol_type=“unassigned DNA” /note=“Substrate for Type I topoisomerase from Plasmodium genus” /organism=“Artificial Sequence”
  misc_feature 1: /note=“Adenine nucleotide which may optionally have a 5′ amino-C6 modification”
actaccattctgagtcgttcgaagttcctatacttt

SEQ ID NO: 2 - DNA, 36 bp, Artificial Sequence
  source 1..36: /mol_type=“unassigned DNA” /note=“Substrate for Type I topoisomerase from Plasmodium genus” /organism=“Artificial Sequence”
  misc_feature 1: /note=“Thymine nucleotide which may optionally have a 5′ amino-C6 modification”
tctagaaagtataggaacttcgaacgactcagaatg

SEQ ID NO: 3 - DNA, 37 bp, Artificial Sequence
  source 1..37: /mol_type=“unassigned DNA” /note=“Substrate for Type I topoisomerase from Plasmodium genus” /organism=“Artificial Sequence”
actaccattctgagtcgttcgatctaaaagacttaga

SEQ ID NO: 4 - DNA, 37 bp, Artificial Sequence
  source 1..37: /mol_type=“unassigned DNA” /note=“Substrate for Type I topoisomerase from Plasmodium genus” /organism=“Artificial Sequence”
atttttctaagtcttttagatcgaacgactcagaatg

SEQ ID NO: 5 - DNA, 42 bp, Artificial Sequence
  source 1..42: /mol_type=“unassigned DNA” /note=“Substrate for Type I topoisomerase from Mycobacterium genus” /organism=“Artificial Sequence”
  misc_feature 42: /note=“Cytosine nucleotide which may optionally have a 3′ amino-C6 modification”
cagtgagcgagcttccgcttgacatcccaatagtttctcttc

SEQ ID NO: 6 - DNA, 29 bp, Artificial Sequence
  source 1..29: /mol_type=“unassigned DNA” /note=“Substrate for HIV integrase” /organism=“Artificial Sequence”
  misc_feature 1: /note=“Thymine nucleotide which may optionally have a 5′ amino-C6 modification”
tttagtcagtgtggaaaatctctagcagt

SEQ ID NO: 7 - DNA, 29 bp, Artificial Sequence
  source 1..29: /mol_type=“unassigned DNA” /note=“Substrate for HIV integrase” /organism=“Artificial Sequence”
  misc_feature 1: /note=“Adenine nucleotide which may optionally have a 5′ amino-C6 modification”
actgctagagattttccacactgactaaa

SEQ ID NO: 8-11 - skipped (4 entries)

SEQ ID NO: 12 - DNA, 76 bp, Artificial Sequence
  source 1..76: /mol_type=“unassigned DNA” /note=“Substrate for an integrase” /organism=“Artificial Sequence”
tgcacgcatgtcgatgtgtcgcaatcgcatgttgtcatcgtgcatgcatcatgacgtgctgactgaagcttccgct

SEQ ID NO: 13 - DNA, 76 bp, Artificial Sequence
  source 1..76: /mol_type=“unassigned DNA” /note=“Substrate for an integrase” /organism=“Artificial Sequence”
tcagcacgtcatgatgcatgcacgatgacaacatgcgattgcgacacatcgacatgcgtgcaagcggaagcttcag

SEQ ID NO: 14 - DNA, 28 bp, Artificial Sequence
  source 1..28: /mol_type=“unassigned DNA” /note=“Topoisomerase substrate” /organism=“Artificial Sequence”
cagtgagcgtctggctgaagcttccgct

SEQ ID NO: 15 - DNA, 35 bp, Artificial Sequence
  source 1..35: /mol_type=“unassigned DNA” /note=“Substrate for Type I topoisomerase from Plasmodium genus” /organism=“Artificial Sequence”
ttctagaccagacgctcactgagcggaagcttcag

SEQ ID NO: 16 - DNA, 28 bp, Artificial Sequence
  source 1..28: /mol_type=“unassigned DNA” /note=“SHOfLE oligonucleotide” /organism=“Artificial Sequence”
  misc_feature 1: /note=“Cytosine nucleotide whcih may optionally have a 5′ biotin modification”
cagtgagcgtctggctgaagcttccgct

SEQ ID NO: 17 - DNA, 28 bp, Artificial Sequence
  source 1..28: /mol_type=“unassigned DNA” /note=“SHOfLE oligonucleotide” /organism=“Artificial Sequence”
  misc_feature 1: /note=“Cytosine nucleotide which may optionally have a 5′ biotin modification”
ccagacgctcactgagcggaagcttcag

SEQ ID NO: 18 - DNA, 2805 bp, Mycobacterium tuberculosis
  source 1..2805: /mol_type=“unassigned DNA” /note=“Mycobacterium tuberculosis topoisomerase I gene sequence” /organism=“Mycobacterium tuberculosis”
ttggctgacccgaaaacgaagggccgtggcagcggcggcaatggcagcggccggcgactggtcatcgtcgagtcgcccaccaaggcgcgcaagctggcctcctacctgggctctggctacatcgtcgagtcctcccgggggcacatccgtgacttgccgcgggccgcgtcggatgtacccgcaaagtacaagtcgcagccgtgggcgcggctcggggtcaacgtcgacgccgacttcgaaccgctctacatcatcagcccggagaaacggagcaccgtcagcgagctcaggggcctgctcaaagacgtggacgagctgtatctggccacggatggggaccgtgagggcgaagctattgcctggcatctgctggaaaccctcaaaccgcgcataccggtaaagcggatggtcttccacgagatcaccgaaccggcgatccgcgccgccgccgagcacccccgcgacctagacatcgacctggtcgacgcgcaggagacccggcgcatcctggaccggctgtacggctacgaagtcagcccagtgctgtggaagaaggtcgcccccaagttgtcggcgggccgggtgcagtcggtggccacccgcatcatcgtggcgcgcgaacgcgaccgcatggcgttccgcagcgcggcctactgggacatccttgccaagctggatgccagcgtgtccgacccggacgccgcgccgcccaccttcagcgcccggctgacggccgtggctggccggcgggtggccactggccgcgatttcgactcgctgggcacgctgcgcaaaggcgacgaagtcattgtgctcgacgaggggagcgcgaccgcgttggccgcgggcctggatggcacgcagctgaccgtggcctcggccgaggagaagccctacgcccggcgcccgtacccgccgttcatgacctccacgctgcagcaagaggccagccgcaagctgcggttctccgccgagcggacgatgagcatcgcccagcggctgtacgaaaacggctacatcacctatatgcgtaccgactccaccacgctgtcggagtcggcgatcaacgccgcacgtacccaggcgcgccagctctacggcgacgagtacgtcgcgccggcgccgcgccaatacacccgcaaggtgaagaacgcccaggaagcgcacgaggctatccggcccgccggtgaaacgtttgccaccccggacgcggtgcgtcgcgaactcgacggtcccaacattgatgatttccggctctatgagctgatttggcaacgcaccgtagcctcgcagatggccgatgcgcggggcatgacgctgagcctgcggatcactggcatgtcggggcaccaggaggtggtgttctccgcgaccggacgcaccttgacgttcccgggcttcctcaaggcctacgtggagaccgtggacgagctggtcggcggcgaggctgacgatgccgagcggcgactgccccatctgaccccgggtcaacggttggacatcgtcgagttgaccccagacggccatgccaccaacccgccggcccgctacaccgaggcgtcgctggtcaaagcgctcgaggagctgggcatcggccgcccgtcgacctactcgtcgatcatcaagaccatccaggatcgcggctacgtgcacaagaagggcagtgcactggtgccgtcatgggtggcgttcgcggtaaccggtctgctcgagcagcatttcggtcggctcgtcgactacgacttcaccgcggcgatggaagacgagctcgacgagatcgccgccggcaacgagcgccgcaccaactggctcaacaacttctactttggtggcgatcacggtgtgcccgattcggtagcccgatcgggtggcctcaagaagcttgtcgggatcaatctcgagggcatcgacgcacgagaagtaaactctatcaagctttttgacgacacccacggacgccccatatatgttcgggtgggcaagaacggtccctacctggaacgtttggtggccggcgacaccggtgagcccacgccgcagcgggccaacctcagcgactcgattaccccggacgagctgactctacaggtggccgaagagctctttgccacaccgcaacagggacggactttgggcttggacccagaaaccggccacgagatcgtggccagggaaggccggtttgggccgtatgtgaccgagatcctgccggagcctgcggctgatgcggccgcggccgctcagggagtcaagaaacgccagaaggccgccgggcccaaaccgcgcaccggttcgttgctgcggagcatggacctacagacggtcaccctcgaagacgcgctgaggctgctgtcactgccgcgcgtggtcggagtggaccccgcctcgggtgaggagatcaccgcgcagaacgggcgctacggaccgtatctaaagcgcggcaacgattctcgatcactggtcaccgaagaccagatattcaccatcacgctcgacgaagccctgaagatctacgcagagccgaaacgtcgtggccggcaaagcgcttcggctccgccgctgcgcgagctgggaacagatccggcgtcgggcaagccaatggtcatcaaggacggccgattcgggccgtacgtcaccgacggtgagaccaatgccagcctgcgtaagggcgacgacgtggcttccataaccgacgagcgcgccgccgagctgttggccgatcgccgagcccggggtccggcaaaacggccagccaggaaagctgcccggaaggtgccggcgaagaaggcagccaagcgcgactag

SEQ ID NO: 19 - protein, 934 aa, Mycobacterium tuberculosis
  Mycobacterium tuberculosis topoisomerase I protein sequence
MADPKTKGRGSGGNGSGRRLVIVESPTKARKLASYLGSGYIVESSRGHIRDLPRAASDVPAKYKSQPWARLGVNVDADFEPLYIISPEKRSTVSELRGLLKDVDELYLATDGDREGEAIAWHLLETLKPRIPVKRMVFHEITEPAIRAAAEHPRDLDIDLVDAQETRRILDRLYGYEVSPVLWKKVAPKLSAGRVQSVATRIIVARERDRMAFRSAAYWDILAKLDASVSDPDAAPPTFSARLTAVAGRRVATGRDFDSLGTLRKGDEVIVLDEGSATALAAGLDGTQLTVASAEEKPYARRPYPPFMTSTLQQEASRKLRFSAERTMSIAQRLYENGYITYMRTDSTTLSESAINAARTQARQLYGDEYVAPAPRQYTRKVKNAQEAHEAIRPAGETFATPDAVRRELDGPNIDDFRLYELIWQRTVASQMADARGMTLSLRITGMSGHQEVVFSATGRTLTFPGFLKAYVETVDELVGGEADDAERRLPHLTPGQRLDIVELTPDGHATNPPARYTEASLVKALEELGIGRPSTYSSIIKTIQDRGYVHKKGSALVPSWVAFAVTGLLEQHFGRLVDYDFTAAMEDELDEIAAGNERRTNWLNNFYFGGDHGVPDSVARSGGLKKLVGINLEGIDAREVNSIKLFDDTHGRPIYVRVGKNGPYLERLVAGDTGEPTPQRANLSDSITPDELTLQVAEELFATPQQGRTLGLDPETGHEIVAREGRFGPYVTEILPEPAADAAAAAQGVKKRQKAAGPKPRTGSLLRSMDLQTVTLEDALRLLSLPRVVGVDPASGEEITAQNGRYGPYLKRGNDSRSLVTEDQIFTITLDEALKIYAEPKRRGRQSASAPPLRELGTDPASGKPMVIKDGRFGPYVTDGETNASLRKGDDVASITDERAAELLADRRARGPAKRPARKAARKVPAKKAAKRD

SEQ ID NO: 20 - DNA, 2520 bp, Plasmodium falciparum
  source 1..2520: /mol_type=“unassigned DNA” /note=“Plasmodium falciparum Gene sequence (ACCESSION NC_004326)” /organism=“Plasmodium falciparum”
atgcaatcaatggaaataaatgataataacagtatcaagaatgaaagtacatctgatgatgatatattaattaataaaattaaacaaaacttgggtaataataaatcatgtaattctagatcttccaaaaaggaatctataaaaaagcaaaagagcaattctgaacttggtataaaaaagaacacaaagaaatcattaggtataaaaaaagaggaagaaaaaaaaaaacaaataagcaaaagaaaaagtaatgaactaaaagaaaaaaataatttgaaagagggaaaaaagaaatatgtggaaaaaaaatctagaacagtaaaagatgaaaccaagttaacgaatgttataaaaaaagaaactcaaaataataagaaacctaaaaaattacttaaaaaatcagaagaaaattttgaaccaataaatagatggtgggaaaaaatagatgatcaaacagatatacaatggaattatttagaacatcgaggattaatattttcccctccatacgttcaacatcatgtaccaattttttataaaagtataaaaattgaattaaatgcaaaatcagaagaattagctacctattggtgtagtgcaattggtagtgattattgtacaaaagaaaagtttatattaaatttttttaaaacatttataaatagtttagaaaatgataatattataaaacaagagaatgaaacgaaattaaaaaaaggagatatatctaattttaagtttattgattttatgccaatcaaagatcatttattaaaattaagagaagaaaagttaaataaaacaaaagaagaaaaagaagaggaaaaaaaaatgagaatggaaaaagaattaccatatacatatgcgttagttgattggattcgtgaaaagatatcaagtaataaagcagaaccacctgggttatttagaggaagaggagaacatccaaaacaaggtttattaaaaaaaagaatttttccagaagatgttgtaattaatattagtaaagatgcacctgtaccacgattatatgataatatgtgtggacataattggggtgatatatatcatgataataaagtaacatggttagcttattataaagatagtataaatgatcaaataaaatatacttttttatctgctcaatcaaaatttaaaggatataaagatcttatgaaatatgaaaatgctcgaaaattaaaatcatgtgttcataaaattagggaagattataaaaataaaatgaaaaataaaaatattattgataaacaattaggaacagctgtttatttaatagattttctagcattaagagtaggaggagaaaaagatatcgatgaagaagcagatactgtaggttgttgtagtttaagagtagaacatattagttttgcacacgatataccttttaaaagtgtagattcaaaagaacaaaaaacaaatgatgaaaaagtaaataaaataccattaccaacaaatttagaaagtatttcatcagaagattgttatataactttagattttttaggaaaagatagtatacgatattttaatacagtcaaaatagataaacaagcatatattaatataataatattttgtaaaaataaaaatagagatgaaggagtttttgatcaaataacttgttcaaaattaaatgaatatctaaaagaaattatgcctactttatcagctaaagtgtttcgtacatataatgcttcaattacattagatcaacaattaaaaagaataaaagaagtttatggaaaaacaacatattcattatattctggtgaaacagaattacacaaatcgaaaaaaagaaaatctagccatttaacttcagatacaaatatattaagtgatgcaagtgattctactattaatgatgtaaataacgagtatgatgaaaatggaataaataaaaaactatcatatgctactactgtaggaaaagaaaatgatgtcgatgataaaaactcaccaatagaagttgacgtttcaaatataaatgaacttattaatttttacaataatgcaaatagagaagtagccatattatgtaaccatcaaagaagtattccaaaacaacatgatacaactatgtcaaaaataaaaaaacaaattgaattatataatgaagatataaaagaatataaaaaatatttgcaacatttaaaaaaaaatagtgataaaaaatttatctttgtttcgaaagtttctactttagatggaactttaagaccaaataaagtcaaagaaaatatgaaagaagaatcttgtaaaaaaaaactaattactcttataaaaaaagttgaattattaaataaccaaatgaaagtaagagatgataataaaactattgctttaggtacatctaaaattaattatatggatccaagaataactgttgctttttgtaaaaaatttgaaatacccatagaaaaagtatttaatagaagtttaagacttaaatttccttgggccatgtttgctacaaaaaattttacattttaa

SEQ ID NO: 21 - protein, 839 aa, Plasmodium falciparum
  Plasmodium falciparum Protein sequence (ACCESSION XP_001351663)
MQSMEINDNNSIKNESTSDDDILINKIKQNLGNNKSCNSRSSKKESIKKQKSNSELGIKKNTKKSLGIKKEEEKKKQISKRKSNELKEKNNLKEGKKKYVEKKSRTVKDETKLTNVIKKETQNNKKPKKLLKKSEENFEPINRWWEKIDDQTDIQWNYLEHRGLIFSPPYVQHHVPIFYKSIKIELNAKSEELATYWCSAIGSDYCTKEKFILNFFKTFINSLENDNIIKQENETKLKKGDISNFKFIDFMPIKDHLLKLREEKLNKTKEEKEEEKKMRMEKELPYTYALVDWIREKISSNKAEPPGLFRGRGEHPKQGLLKKRIFPEDVVINISKDAPVPRLYDNMCGHNWGDIYHDNKVTWLAYYKDSINDQIKYTFLSAQSKFKGYKDLMKYENARKLKSCVHKIREDYKNKMKNKNIIDKQLGTAVYLIDFLALRVGGEKDIDEEADTVGCCSLRVEHISFAHDIPFKSVDSKEQKTNDEKVNKIPLPTNLESISSEDCYITLDFLGKDSIRYFNTVKIDKQAYINIIIFCKNKNRDEGVFDQITCSKLNEYLKEIMPTLSAKVFRTYNASITLDQQLKRIKEVYGKTTYSLYSGETELHKSKKRKSSHLTSDTNILSDASDSTINDVNNEYDENGINKKLSYATTVGKENDVDDKNSPIEVDVSNINELINFYNNANREVAILCNHQRSIPKQHDTTMSKIKKQIELYNEDIKEYKKYLQHLKKNSDKKFIFVSKVSTLDGTLRPNKVKENMKEESCKKKLITLIKKVELLNNQMKVRDDNKTIALGTSKINYMDPRITVAFCKKFEIPIEKVFNRSLRLKFPWAMFATKNFTF

SEQ ID NO: 22 - DNA, 30 bp, Artificial Sequence
  source 1..30: /mol_type=“unassigned DNA” /note=“Substrate for MLV integrase” /organism=“Artificial Sequence”
ttgactacccgtcagcgggggtctttcatt

SEQ ID NO: 23 - DNA, 30 bp, Artificial Sequence
  source 1..30: /mol_type=“unassigned DNA” /note=“Substrate for MLV integrase” /organism=“Artificial Sequence”
aatgaaagacccccgctgacgggtagtcaa

SEQ ID NO: 24 - skipped

SEQ ID NO: 25 - DNA, 68 bp, Artificial Sequence
  source 1..68: /mol_type=“unassigned DNA” /note=“PCR primer P1” /organism=“Artificial Sequence”
atttttctaagtcttttagatcgaacgactcagaatgatgcatgtatactaaactcacaaattagagc

SEQ ID NO: 26 - DNA, 46 bp, Artificial Sequence
  source 1..46: /mol_type=“unassigned DNA” /note=“PCR primer P2” /organism=“Artificial Sequence”
tttttttttttttttttttttttttgctttctcatagctcacgctg

SEQ ID NO: 27 - protein, 706 aa, Streptococcus agalactiae
  Streptococcus agalactiae DNA topoisomerase I amino acid sequence (ACCESSION WP_000246605)
MATTTKTSTKKTSKKKSATAKKNLVIVESPAKAKTIEKYLGRNYKVVASVGHIRDLKKSSMSIDFENNYEPQYINIRGKGPLINDLKKEAKKAKKVYLASDPDREGEAISWHLAHILDLDKEDRNRVVFNEITKDAVKNAFVEPRQINMDLVDAQQARRVLDRIVGYSISPILWKKVKKGLSAGRVQSVALKLIIDRENEIKAFQPEEYWTIDGSFKKGTRKFNATFYGLDGKKFKLSNNEDVKTVLKRIKTDEFLVEKVEKKERPRNAPLPYTTSSLQQDAANKINFRTRKTMMIAQQLYEGLSLGTAGHQGLITYMRTDSTRISPLAQNEAAEFITNRFGANYSKHGNKVKNASGAQDAHEAIRPSSVNHTPESIAKYLDKDQLKLYTLIWNRFIASQMTAAVFDTMKVNLTQNGVTFIANGSQVKFDGYMAVYNDTDKNKMLPDMEEGESVKKVNTNPEQHFTQPPARFSEASLIKTLEENGVGRPSTYAPTLETIQKRYYVKLAAKRFEPTELGEIVNSLIVEFFPDIVDVTFTAEMEGKLDEVEIGKEQWQKIIDEFYKPFEKELAKAETEMEKIQIKDEPAGFDCELCGSPMVIKLGRYGKFYACSNFPECHNTKAITKEIGVICPICQKGQVIERKTKRNRIFYGCDRYPECEFTSWDKPIGRTCPKSNDFLVEKKVRGGGKQVVCSNEKCDYQEEKIK

SEQ ID NO: 28 - DNA, 28 bp, Streptococcus agalactiae
  source 1..28: /mol_type=“unassigned DNA” /note=“Substrate for Type I topoisomerase from Streptococcus genus” /organism=“Streptococcus agalactiae”
acttcgggatgctgaaacgcagctgtgc